SEQUENCE LISTING (81 sequences)

SEQ ID NO: 1 - RNA, 21 nt, Artificial Sequence
  Description of Artificial Sequence Synthetic oligonucleotide
ccuacgccgaguacuucgacu

SEQ ID NO: 2 - RNA, 21 nt, Artificial Sequence
  Description of Artificial Sequence Synthetic oligonucleotide
ucgaaguacucggcguaggua

SEQ ID NO: 3 - protein, 4 aa, Artificial Sequence
  Description of Artificial Sequence Synthetic peptide
GKKK

SEQ ID NO: 4 - protein, 5 aa, Artificial Sequence
  Description of Artificial Sequence Synthetic peptide
AGKKK

SEQ ID NO: 5 - protein, 6 aa, Artificial Sequence
  Description of Artificial Sequence Synthetic peptide
AGGKKK

SEQ ID NO: 6 - protein, 5 aa, Artificial Sequence
  Description of Artificial Sequence Synthetic peptide
GKKKK

SEQ ID NO: 7 - protein, 6 aa, Artificial Sequence
  Description of Artificial Sequence Synthetic peptide
GGKKKK

SEQ ID NO: 8 - protein, 6 aa, Artificial Sequence
  Description of Artificial Sequence Synthetic peptide
AGKKKK

SEQ ID NO: 9 - protein, 4 aa, Artificial Sequence
  Description of Artificial Sequence Synthetic peptide
GRRR

SEQ ID NO: 10 - protein, 8 aa, Artificial Sequence
  Description of Artificial Sequence Synthetic peptide
GRRRGKKK

SEQ ID NO: 11 - protein, 4 aa, Artificial Sequence
  Description of Artificial Sequence Synthetic peptide
KKKG

SEQ ID NO: 12 - protein, 5 aa, Artificial Sequence
  Description of Artificial Sequence Synthetic peptide
KKKGG

SEQ ID NO: 13 - protein, 5 aa, Artificial Sequence
  Description of Artificial Sequence Synthetic peptide
KKKKG

SEQ ID NO: 14 - protein, 4 aa, Artificial Sequence
  Description of Artificial Sequence Synthetic peptide
RRRG

SEQ ID NO: 15 - protein, 4 aa, Artificial Sequence
  Description of Artificial Sequence Synthetic peptide
GKKK

SEQ ID NO: 16 - protein, 4 aa, Artificial Sequence
  Description of Artificial Sequence Synthetic peptide
GGKK

SEQ ID NO: 17 - protein, 5 aa, Artificial Sequence
  Description of Artificial Sequence Synthetic peptide
GGKKK

SEQ ID NO: 18 - protein, 5 aa, Artificial Sequence
  Description of Artificial Sequence Synthetic peptide
GGKGK

SEQ ID NO: 19 - protein, 7 aa, Artificial Sequence
  Description of Artificial Sequence Synthetic peptide
GGKKKKK

SEQ ID NO: 20 - protein, 7 aa, Artificial Sequence
  Description of Artificial Sequence Synthetic peptide
GGKKKGK

SEQ ID NO: 21 - protein, 8 aa, Artificial Sequence
  Description of Artificial Sequence Synthetic peptide
GGKKKKKK

SEQ ID NO: 22 - protein, 9 aa, Artificial Sequence
  Description of Artificial Sequence Synthetic peptide
GGKKKKKKG

SEQ ID NO: 23 - protein, 10 aa, Artificial Sequence
  Description of Artificial Sequence Synthetic peptide
GGKKKGKKKK

SEQ ID NO: 24 - protein, 9 aa, Artificial Sequence
  Description of Artificial Sequence Synthetic peptide
GGKKKKKKA

SEQ ID NO: 25 - protein, 9 aa, Artificial Sequence
  Description of Artificial Sequence Synthetic peptide
GGKKKKKKH

SEQ ID NO: 26 - protein, 10 aa, Artificial Sequence
  Description of Artificial Sequence Synthetic peptide
GGKKKKKKKK

SEQ ID NO: 27 - protein, 11 aa, Artificial Sequence
  Description of Artificial Sequence Synthetic peptide
GGKGKGKGKGK

SEQ ID NO: 28 - protein, 11 aa, Artificial Sequence
  Description of Artificial Sequence Synthetic peptide
GGKKKKKKKKK

SEQ ID NO: 29 - protein, 12 aa, Artificial Sequence
  Description of Artificial Sequence Synthetic peptide
GGKKKKKKKGRG

SEQ ID NO: 30 - protein, 13 aa, Artificial Sequence
  Description of Artificial Sequence Synthetic peptide
GGKKKKKKGKKKK

SEQ ID NO: 31 - protein, 7 aa, Artificial Sequence
  Description of Artificial Sequence Synthetic peptide
GGKKKKK

SEQ ID NO: 32 - protein, 12 aa, Artificial Sequence
  Description of Artificial Sequence Synthetic peptide
GGKKKKKGKKKK

SEQ ID NO: 33 - protein, 5 aa, Artificial Sequence
  Description of Artificial Sequence Synthetic peptide
GPKKK

SEQ ID NO: 34 - protein, 12 aa, Artificial Sequence
  Description of Artificial Sequence Synthetic peptide
CYGRKKRRQRRR

SEQ ID NO: 35 - protein, 20 aa, Artificial Sequence
  Description of Artificial Sequence Synthetic peptide
CKKKGKKKGKKKGKKKGKKK

SEQ ID NO: 36 - protein, 4 aa, Artificial Sequence
  Description of Artificial Sequence Synthetic peptide
KGKG

SEQ ID NO: 37 - protein, 5 aa, Artificial Sequence
  Description of Artificial Sequence Synthetic peptide
KKKKG

SEQ ID NO: 38 - protein, 6 aa, Artificial Sequence
  Description of Artificial Sequence Synthetic peptide
KGKGKG

SEQ ID NO: 39 - protein, 4 aa, Artificial Sequence
  Description of Artificial Sequence Synthetic peptide
GRGK

SEQ ID NO: 40 - protein, 4 aa, Artificial Sequence
  Description of Artificial Sequence Synthetic peptide
KKKK

SEQ ID NO: 41 - protein, 5 aa, Artificial Sequence
  Description of Artificial Sequence Synthetic peptide
KKKKK

SEQ ID NO: 42 - protein, 11 aa, Artificial Sequence
  Description of Artificial Sequence Synthetic peptide
KGKKKKGKKKK

SEQ ID NO: 43 - protein, 11 aa, Artificial Sequence
  Description of Artificial Sequence Synthetic peptide
KGKKKKGKKKK

SEQ ID NO: 44 - protein, 11 aa, Artificial Sequence
  Description of Artificial Sequence Synthetic peptide
KGKKKKGKKKK

SEQ ID NO: 45 - protein, 11 aa, Artificial Sequence
  Description of Artificial Sequence Synthetic peptide
KGKKKKGKKKK

SEQ ID NO: 46 - protein, 11 aa, Artificial Sequence
  Description of Artificial Sequence Synthetic peptide
KGKKKKGKKKK

SEQ ID NO: 47 - protein, 8 aa, Artificial Sequence
  Description of Artificial Sequence Synthetic peptide
KKKGKKKK

SEQ ID NO: 48 - protein, 6 aa, Artificial Sequence
  Description of Artificial Sequence Synthetic peptide
KKKKGG

SEQ ID NO: 49 - protein, 10 aa, Artificial Sequence
  Description of Artificial Sequence Synthetic peptide
PGKKKGKKKK

SEQ ID NO: 50 - protein, 5 aa, Artificial Sequence
  Description of Artificial Sequence Synthetic peptide
GGKKK

SEQ ID NO: 51 - protein, 5 aa, Artificial Sequence
  Description of Artificial Sequence Synthetic peptide
GGRRR

SEQ ID NO: 52 - protein, 9 aa, Artificial Sequence
  Description of Artificial Sequence Synthetic peptide
KLKLKLKLK

SEQ ID NO: 53 - protein, 18 aa, Artificial Sequence
  Description of Artificial Sequence Synthetic peptide
KLKLKLKLKKLKLKLKLK

SEQ ID NO: 54 - protein, 6 aa, Artificial Sequence
  Description of Artificial Sequence Synthetic peptide
RRRRRR

SEQ ID NO: 55 - protein, 11 aa, Artificial Sequence
  Description of Artificial Sequence Synthetic peptide
KKAKAKAKAKA

SEQ ID NO: 56 - protein, 11 aa, Artificial Sequence
  Description of Artificial Sequence Synthetic peptide
KKFKFKFKFKF

SEQ ID NO: 57 - protein, 11 aa, Artificial Sequence
  Description of Artificial Sequence Synthetic peptide
KKIKIKIKIKI

SEQ ID NO: 58 - protein, 11 aa, Artificial Sequence
  Description of Artificial Sequence Synthetic peptide
KKKLKKLKKLK

SEQ ID NO: 59 - protein, 15 aa, Artificial Sequence
  Description of Artificial Sequence Synthetic peptide
KKAKAKAKAKAKAKA

SEQ ID NO: 60 - protein, 15 aa, Artificial Sequence
  Description of Artificial Sequence Synthetic peptide
KKFKFKFKFKFKFKF

SEQ ID NO: 61 - protein, 14 aa, Artificial Sequence
  Description of Artificial Sequence Synthetic peptide
KKKLKKLKKLKKKK

SEQ ID NO: 62 - protein, 9 aa, Artificial Sequence
  Description of Artificial Sequence Synthetic peptide
KLKLKLKLK

SEQ ID NO: 63 - protein, 10 aa, Artificial Sequence
  Description of Artificial Sequence Synthetic peptide
AKAKAKAKLK

SEQ ID NO: 64 - protein, 10 aa, Artificial Sequence
  Description of Artificial Sequence Synthetic peptide
FKFKFKFKFK

SEQ ID NO: 65 - protein, 10 aa, Artificial Sequence
  Description of Artificial Sequence Synthetic peptide
IKIKIKIKIK

SEQ ID NO: 66 - protein, 10 aa, Artificial Sequence
  Description of Artificial Sequence Synthetic peptide
KLKKLKKLKK

SEQ ID NO: 67 - protein, 14 aa, Artificial Sequence
  Description of Artificial Sequence Synthetic peptide
AKAKAKAKAKAKAK

SEQ ID NO: 68 - protein, 14 aa, Artificial Sequence
  Description of Artificial Sequence Synthetic peptide
FKFKFKFKFKFKFK

SEQ ID NO: 69 - protein, 11 aa, Artificial Sequence
  Description of Artificial Sequence Synthetic peptide
CKKKKKKKKKK

SEQ ID NO: 70 - protein, 11 aa, Artificial Sequence
  Description of Artificial Sequence Synthetic peptide
CKKKKKKKKKK

SEQ ID NO: 71 - protein, 11 aa, Artificial Sequence
  Description of Artificial Sequence Synthetic peptide
CKKKKKKKKKK

SEQ ID NO: 72 - protein, 5 aa, Artificial Sequence
  Description of Artificial Sequence Synthetic peptide
KGRRR

SEQ ID NO: 73 - protein, 5 aa, Artificial Sequence
  Description of Artificial Sequence Synthetic peptide
KGRRR

SEQ ID NO: 74 - protein, 6 aa, Artificial Sequence
  Description of Artificial Sequence Synthetic peptide
KKLKLK

SEQ ID NO: 75 - protein, 10 aa, Artificial Sequence
  Description of Artificial Sequence Synthetic peptide
KKLKLKLKLK

SEQ ID NO: 76 - protein, 5 aa, Artificial Sequence
  Description of Artificial Sequence Synthetic peptide
KGRRR

SEQ ID NO: 77 - protein, 7 aa, Artificial Sequence
  Description of Artificial Sequence Synthetic peptide
KRRRRRR

SEQ ID NO: 78 - protein, 10 aa, Artificial Sequence
  Description of Artificial Sequence Synthetic peptide
KKLKLKLKLK

SEQ ID NO: 79 - protein, 5 aa, Artificial Sequence
  Description of Artificial Sequence Synthetic peptide
KLKLK

SEQ ID NO: 80 - protein, 10 aa, Artificial Sequence
  Description of Artificial Sequence Synthetic peptide
AKAKAKAKAK

SEQ ID NO: 81 - protein, 13 aa, Artificial Sequence
  Description of Artificial Sequence Synthetic peptide
KKKKLKKLKKLKK

(19) United States
(12) Patent Application Publication
(10) Pub. No.: US 2010/0056475 A1
(43) Pub. Date: Mar. 4, 2010

(54) CYCLODEXTRIN CONJUGATES

(76) Inventors: Alexander Chucholowski, San Diego, CA (US); Thomas Hermann, Cardiff by the Sea, CA (US); Benjamin Ayida, Chula Vista, CA (US); Tingmin Wang, San Diego, CA (US); Alisher Khasanov, San Diego, CA (US)

Correspondence Address: FOLEY & LARDNER LLP, P.O. BOX 80278, SAN DIEGO, CA, 92138-0278, US

(21) Appl. No.: 12/536,455

(22) Filed: Aug. 5, 2009

Related U.S. Application Data
(60) Provisional application No. 61/086,776, filed on Aug. 6, 2008.

(51) Int. Cl. A61K 31/724; C08B 37/16; C07K 9/00; C07H 21/00; C12N 5/00; C07H 1/00; A61P 43/00

(52) U.S. Cl. 514/58; 536/103; 530/322; 536/23.1; 435/375; 536/25.3; 536/124

(57) ABSTRACT

It has been discovered that the uptake of anionic charged species into cells can be enhanced by noncovalently associating such species with specifically modified forms of cyclodextrin. The invention modified forms of cyclodextrin form well defined stoichiometric complexes with anionic charged molecules. This discovery enables one to produce various compositions containing anionic charged molecules and facilitates methods for enhancing the cellular uptake of double-stranded or hairpin nucleic acid.

### RELATED APPLICATION

This application claims benefit of priority from U.S. provisional application Ser. No. 61/086,776 filed Aug. 6, 2008 entitled “Cyclodextrin Conjugates” which is incorporated by reference herein in its entirety.

### TECHNICAL FIELD

This invention relates to cyclodextrin conjugates and compositions containing same. Invention conjugates are useful, for example, for drug delivery and therapeutic treatment of diseases, and in particular, for delivery of siRNA and therapeutics.

### BACKGROUND

RNA interference (RNAi) is an evolutionarily conserved process by which double-stranded small interfering RNA (siRNA) of 19-21 base pairs of oligonucleotides guide a cellular RNA-cleaving protein complex (RISC) to sequence-complementary target sites at messenger RNA (mRNA). Unlike other mRNA targeting strategies, RNAi takes advantage of the physiological gene silencing machinery. Through control of the dose of siRNA, gene expression can be shut off completely (“knock out”) or only down-regulated (“knock down”), which renders RNAi highly attractive to target genes of therapeutic importance. RNAi can be achieved by either delivery of synthetic siRNAs or endogenous expression of small hairpin RNA, siRNA, and microRNA (miRNA). Thus, the potential use of siRNA as a therapeutic agent has attracted great attention as a novel approach for treating severe and chronic diseases. However, because of the difficulty of delivering highly charged siRNA into target cells, the potential of this powerful therapeutic has not been greatly realized.

Although the exact mechanism for the uptake of highly anionic charged oligonucleotides by cells has not been fully elucidated, oligonucleotide uptake is believed to be a sequence-independent, saturable process and may he dependent on temperature and energy. Unlike the extensively tested delivery vehicles for antisense RNA, the vehicles and/or methods for delivery of siRNA remain limited.

### SUMMARY OF INVENTION

In accordance with the present invention, it has been discovered that the uptake of anionic charged species into cells can be enhanced by noncovalently associating such species with specifically modified forms of cyclodextrins. The invention modified forms of cyclodextrin form well defined stoichiometric complexes with anionic charged molecules. This discovery enables one to produce various compositions containing anionic charged molecules and facilitates enhanced cellular uptake of double-stranded or hairpin nucleic acid.

### BRIEF DESCRIPTION OF THE FIGURE

FIG. 1 compares the luciferase expression promoted by a test compound complexed with the luciferase knockdown sequence versus the luciferase expression promoted by the same test compound complexed with the scrambled knockdown sequence. In the figure, empty bars represent luc52/53tt (25 pmol), shaded bars represent 54/55tt dicer (25 pmol) and blackened bars represent % knockdown.

### DETAILED DESCRIPTION OF INVENTION

The present invention relates, at least in part, to biocompatible constructs that have the ability to interact with anionic charged molecules, e.g., siRNA. Invention constructs are based on cyclodextrins that include cationic arms covalently bound thereto via linkers.

In some embodiments, the present invention provides constructs represented by formula I:

CA¹-L¹-CD-L²-CA² (I)

wherein:

CD=cyclodextrin;

L¹, L²=linker; and

CA¹, CA²=cationic arm.

Cyclodextrins (CDs), are a group of cyclic polysaccharides comprising six to eight naturally occurring D(+)-glucopyranose units in alpha-(1,4) linkage. The numbering of the carbon atoms of D(+)-glucopyranose units is illustrated below.

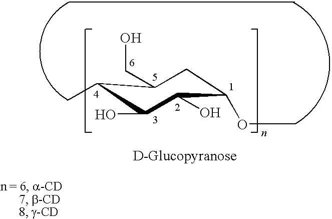

CDs are classified by the number of glucose units they contain: α-cyclodextrin has six glucose units; β-cyclodextrin has seven; and γ-cyclodextrin has eight. Each glucopyranose unit is referred to as ring A, ring B, etc., as exemplified below for β-CD.

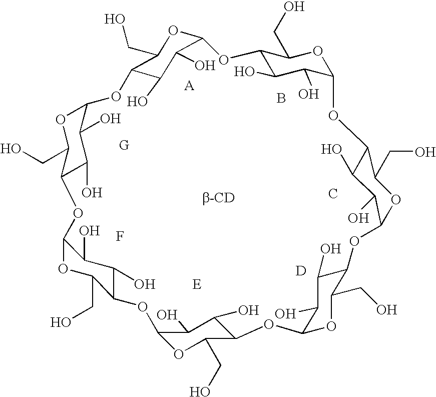

The three-dimensional architecture of CDs consists of cup-like shapes with relatively polar exteriors and apolar interiors. The resulting structure is thought to be able to imbibe hydrophobic compounds to form host-guest complexes with a variety of compounds. (See Wenz, G. Angew Chem. 1994, 106, 851-870.) This property has been extensively utilized to change the physicopharmaceutical properties of lipophilic drugs, e.g., water solubility, bioavailability and improved stability. Consequently, CDs are widely used as transport-active additives. According to both in vitro and in vivo studies, CDs, especially alkylated CD derivatives, may have enhancer activity on transport through cell membranes. For example, Agrawal et al. (U.S. Pat. No. 5,691,316) describes a composition including an oligonucleotide complexed with a CD to achieve enhanced cellular uptake of oligonucleotide.

Cyclodextrins contemplated for use in the practice of the present invention may be any available CDs, e.g., alpha, beta or gamma cyclodextrin. Any appropriate linker to facilitate linkage between a glucopyranose moiety of cyclodextrin to cationic arms can be employed; such linkage can readily be accomplished by known procedures. In some embodiments, each linker of the construct is independently selected from the group consisting of a covalent bond, a disulfide linkage, a protected disulfide linkage, an ether linkage, a thioether linkage, a sulfoxide linkage, a sulfonate linkage, an amine linkage, a hydrazone linkage, a sulfonamide linkage, an urea linkage, an ester linkage, an amide linkage, a carbamate linkage, a dithiocarbamate linkage, and the like, as well as combinations thereof. The linker may be covalently linked at any available positions, e.g. at the 6-position of A,D-rings, A,C-rings or A,E-rings of cyclodextrin.

The specific linkers used in the present invention are selected based on the desired length of the linkers and the chemistry employed for CD derivatization. Linkers with more than one possible orientation for attachment to CD should be understood to embrace all possible orientations for attachment. For example, an ester linkage at the 6 position of glucose can be linked via hydroxy (—OC(O)—) or via oxo (—C(O)O—) moiety; a sulfonate linkage may be linked via hydroxy (—OS(O)₂—) or via mercapto (—S(O)₂O—) moiety; a thiocarbamate linkage may be linked via hydroxy (—OC(S)NH—) or via amino (—NHC(S)O—) moiety. A skilled artisan can readily identify other suitable linkers for attachment of each cationic arm.

In some embodiments, each cationic arm of the constructs comprises a plurality of residues selected from amines, guanidines, amidines, N-containing heterocycles, or combinations thereof. In related embodiments, each cationic arm may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof In related embodiments, one or both of the cationic arms may further comprise neutral and/or polar functional groups, for example, PEGs or fatty acids (either as part of the backbone of the cationic arms or as an substituent thereon). In preferred embodiments, each cationic arm comprises an oligomer independently selected from the group consisting of oligopeptide, oligoamide, cationically functionalized oligoether, cationically functionalized oligosaccharide, oligoamine, oligoethyleneimine, and the like, as well as combinations thereof. The oligomers may be oligopeptides where all the amino acid residues of the oligopeptide are capable of forming positive charges. In some embodiments, the length of the contiguous backbone of each cationic arm is about 12 to about 200 Angstroms; preferably about 12 to about 100 Angstroms. In some embodiments, the oligopeptides may comprise 3 to 50 amino acids; preferably 3 to 40 amino acids; more preferably 6-30 amino acids. In certain preferred embodiments, cyclodextrin of the construct is beta-cyclodextrin and each linker is covalently linked to the 6-position of A,D-rings of beta-cyclodextrin.

As used herein, the term “about” refers to ±10% of a given measurement.

As used herein, the term “amino acids” includes the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to embrace both the (D) and (L) stereoisomers.

As used herein, the term “cationically functional monosaccharides” may include any amine-containing monosaccharide such as glucosamine, galactosamine and 2-amino-sialic acid. It may also include any natural or unnatural derivatized monosaccharides containing one or more functional groups that can form positive charge, e.g. amine and phosphorus containing groups.

As used herein, the term “cationically functionalized oligosaccharide” refers to an oligosaccharide comprising one or more “cationically functional monosaccharides.”

As used herein, the term “cationically functionalized ethylene glycols” may include any substituted ethylene glycols where the substituents comprise functional groups that can form positive charge, e.g. amine and phosphorus containing groups.

As used herein, the term “cationically functionalized oligoether” may include any substituted oligoether where the substituents comprise functional groups that can form positive charge, e.g. amine and phosphorus containing groups.

In some embodiments, invention constructs may further comprise a bio-recognition molecule. In certain aspects, the bio-recognition molecule could be covalently linked or non-covalently linked to the construct. The bio-recognition molecules optionally incorporated into the construct may be any molecules such as oligopeptides or oligosaccharides that are involved in a large range of biological processes including cell attachment, cell penetration and cell recognition so as to promote binding of, recognition of or cell penetration of such molecules. Examples of such bio-recognition molecules include peptidyl-cyclodextrins which can be found in Pean et al. J. Chem. Soc. Perkin Trans. 2, 2000, 853-863. Exemplary molecules include TAT peptides (Transacting Activator of Transcription peptide), linear or cyclic RGD (Arg-Gly-Asp) peptides or RGD peptide mimetics.

In other embodiments, the present invention provides complexes comprising a construct associated with an anionic charged molecule. The anionic charged molecules may be double-stranded or hairpin nucleic acids. In certain embodiments, the anionic charged molecules may be selected from the group consisting of single-stranded DNA, double-stranded DNA, single-stranded RNA, double-stranded RNA, and oligonucleotide comprising non-natural monomers including but not limited to 2′-methoxy or 2′-fluoro-modified nucleotides with ribo- or arabino-stereochemistry at the 2′-position, or thio-substituted phosphate groups. The single-stranded RNA may be mRNA or miRNA. The double-stranded RNA may be siRNA. In further embodiments, the cationic arms are oligopeptides. The length of contiguous backbone of the oligopeptide may be about one third to one half of the length of the contiguous backbone of the anionic charged molecule.

As used herein, the term “nucleic acids” refers to oligonucleotides consisting of deoxyribonucleic acid (DNA) or ribonucleic acid (RNA), or chimeric oligonucleotides, containing DNA and RNA, or oligonucleotide strands containing non-natural monomers, including 2′-methoxy or 2′-fluoro-modified nucleotides with ribo- or arabino-stereochemistry at the 2′-position, or thio-substituted phosphate groups or the like. Nucleic acids contemplated for use in the practice of the present invention may also include conjugated nucleic acids where nucleic acids conjugate to protein, polypeptide or any organic molecules.

As used herein, “double-stranded nucleic acids (hybrids)” are formed from two individual oligonucleotide strands of substantially identical length and complete or near-complete sequence complementarity (“blunt end hybrids”) or offset sequence complementarity (“symmetrical overhang hybrids”, not necessarily implying sequence identity of the overhanging monomers), or from strands of different lengths and complete or offset sequence complementarity (“overhang hybrids”). Preferred length of oligonucleotides in double-stranded nucleic acids is between 15-60 monomers (nucleotides); more preferred are oligonucleotide lengths between 15-45 monomers; even more preferred are oligonucleotide lengths between 19-30 monomers; most preferred are oligonucleotide lengths between 21-27 monomers.

As used herein, “sequence complementarity” is defined as the ability of monomers in two oligonucleotides to form base pairs between one nucleotide in one strand and another nucleotide in the second strand by formation of one or more hydrogen bonds between the monomers in the base pair.

As used herein, “complete sequence complementarity” means that each residue in a consecutive stretch of monomers in two oligonucleotides participates in base pair formation.

As used herein, “near-complete sequence complementarity” means that a consecutive stretch of base pairs is disrupted by no greater than one unpaired nucleotide per 3 consecutive monomers involved in base pairing. Preferably, base pairing refers to base pairs between monomers that follow the Watson-Crick rule (adenine-thymine, A-T; adenine-uracil, A-U; guanine-cytosine, G-C) or form a wobble pair (guanine-uracil, G-U).

As used herein, “hairpin nucleic acids” are formed from a single oligonucleotide strand that has complete or near-complete sequence complementarity or offset sequence complementarity between stretches of monomers within the 5′ and 3′ region such that, upon formation of intra-oligonucleotide base pairs, a hairpin structure is formed that consists of a double-stranded (hybridized) domain and a loop domain which contains nucleotides that do not participate in pairing according to the Watson-Crick rule. Preferred length of hairpin oligonucleotides is between 15-70 monomers (nucleotides); more preferred are hairpin oligonucleotide lengths between 18-55 monomers; even more preferred are hairpin oligonucleotide lengths between 20-35 monomers; most preferred are hairpin oligonucleotide lengths between 21-23 monomers. A skilled artisan will realize nucleotides at the extreme 5′ and 3′ termini of the hairpin may but do not have to participate in base pairing.

In some embodiments, the ratio of the construct to the anionic charged molecule of the complex may range from about 1:1 to about 10:1; preferably from about 1:1 to about 4:1. In further embodiments, the complexes comprise siRNA and the construct of formula I, wherein:
CD is beta-cyclodextrin,
L¹, L² are linkers covalently linked to the 6-positions of A,D-rings of beta-cyclodextrin and

CA¹, CA²independently comprise oligopeptides.

In other embodiments, the present invention provides compositions comprising a pharmaceutical excipient, an anionic charged molecule and a construct of formula I, or a pharmaceutically acceptable ester, salt, or hydrate thereof. The constructs may optionally comprise one or more bio-recognition molecules covalently linked or non-covalently linked to the constructs. Each linker of the constructs may be independently selected from the group consisting of a covalent bond, a disulfide linkage, a protected disulfide linkage, an ether linkage, a thioether linkage, a sulfoxide linkage, an amine linkage, a hydrazone linkage, a sulfonamide linkage, an urea linkage, a sulfonate linkage, an ester linkage, an amide linkage, a carbamate linkage, a dithiocarbamate linkage, and the like, as well as combinations thereof. The linkers may be covalently linked to the 6-positions of A,D-rings, A,C-rings or A,E-rings of cyclodextrin.

In some embodiments, the present invention provides compositions comprising a pharmaceutical excipient and complexes comprising a construct of formula I associated with an anionic charged molecule, or a pharmaceutically acceptable ester, salt, or hydrate thereof. The ratio of the construct to the anionic charged molecule of the complex may range from about 1:1 to about 10:1; preferably from about 1:1 to about 4:1. The anionic charged molecules may be double-stranded or hairpin nucleic acids. The anionic charged molecules may be selected from the group consisting of single-stranded DNA, double-stranded DNA, single-stranded RNA, double-stranded RNA, and oligonucleotide comprising non-natural monomers. The single-stranded DNA, double-stranded DNA, single-stranded RNA and double-stranded RNA may include nucleotides bound to small molecules. In related embodiments, the single-stranded RNAs may be mRNA or miRNA and double-stranded RNA may be siRNA. In more preferred embodiments, the compositions may comprise a pharmaceutical excipient and a complex comprising siRNA and the construct of formula I, wherein:
CD is beta-cyclodextrin;
L¹, L² are linkers covalently linked to the 6-positions of A,D-rings of beta-cyclodextrin; and
CA¹, CA² independently comprise an oligopeptide.

As used herein, the term “pharmaceutical excipient” refers to an inert substance added to a pharmacological composition to further facilitate administration of molecular entities. Examples of pharmaceutical excipients include but are not limited to, calcium carbonate, calcium phosphate, various sugars and types of starch, cellulose derivatives, gelatin, vegetable oils and polyethylene glycols excipient.

As used herein, “pharmaceutically acceptable” refers to materials and compositions that are physiologically tolerable and do not typically produce an allergic or similar untoward reaction, such as gastric upset, dizziness, and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutical acceptable ester” within the context of the present invention represents an ester of a construct of the invention having a carboxy group, preferably a carboxylic acid prodrug ester that may be convertible under physiological conditions to the corresponding free carboxylic acid.

As used herein, the term “pharmaceutically acceptable salt” includes salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

The compositions according to the present invention may be administered to humans and other animals for therapy as either a single dose or in multiple doses. The compositions of the present invention may be administered either as individual therapeutic agents or in combination with other therapeutic agents. The treatments of the present invention may be combined with conventional therapies, which may be administered sequentially or simultaneously. In some embodiments, routes of administration include those selected from the group consisting of oral, intravesically, intravenous, intraarterial, intraperitoneal, local administration, and the like. Intravenous administration is the preferred mode of administration. It may be accomplished with the aid of an infusion pump.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material by a route which does not introduce the compound, drug or other material directly into the central nervous system (for example, subcutaneous administration), such that it enters the patient's system and, thus, is subject to metabolism and other like processes.

Actual dosage levels of the active ingredients in the compositions of the present invention may he varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compositions being employed, the duration of the treatment, other drugs, compounds and/or materials used in combination with the particular therapeutic employed, the age, sex, weight, condition, general health and prior medical history of the patient being treated, and like factors well known in the medical arts.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compositions of the present invention for a patient will range from about 0.0001 to about 100 mg per kilogram of body weight per day.

In other embodiments, the present invention provides methods of attenuating expression of a target gene in treated cells comprising delivering a construct of formula I and a double-stranded or hairpin nucleic acid to said cell in need thereof. Methods of attenuating expression of a target gene in treated cells may also comprise delivery of a complex of formula I associated with a charged molecule to said cell a subject in need thereof.

In yet other embodiments, the present invention provides methods for delivering an anionic charged molecule to a cell, the method comprising:

a) binding non-covalently a construct of formula I to said anionic charged molecule to form a complex; and

b) contacting said cell with said complex; wherein said anionic charged molecule is taken up by said cell.

In certain embodiments, the present invention provides methods for delivering an anionic charged molecule to a cell, said method comprising contacting said cell with a complex prepared by binding non-covalently a construct of formula I to said anionic charged molecule, wherein said anionic charged molecule is taken up by said cell. In preferred embodiments, the charged molecule is siRNA.

In other embodiments, the present invention provides methods for delivering an anionic charged molecule such as siRNA to a cell via local administration to relevant tissues or cells. In yet other embodiments, the present invention provides methods for delivering an anionic charged molecule such as siRNA to a cell via systemic administration (such as via intravenous or subcutaneous administration of siNA) to relevant tissues or cells, such as tissues or cells involved in the maintenance or development of certain diseases in a subject or organism. The methods for delivering an anionic charged molecule such as siRNA can he combined with other therapeutic treatments and modalities as are known in the art for the treatment of or prevention of certain diseases in a subject or organism.

In some embodiments, the present invention provides methods for stabilizing an anionic charged molecule in vivo, said methods comprising contacting said anionic charged molecule with a construct of formula I; in this embodiment, a preferred anionic charged molecule is siRNA.

In other embodiments, the present invention provides methods for increasing the temperature of hybrid dissociation of a double-stranded or hairpin nucleic acid, said methods comprising contacting said nucleic acid with a construct of formula I.

In yet other embodiments, the present invention provides methods for reducing the susceptibility of a double-stranded or hairpin nucleic acid to digestion by enzymatic nuclease, said methods comprising contacting said nucleic acid with a construct of formula I. The nuclease may be an exonuclease or an endonuclease.

In still other embodiments, the present invention provides methods for reducing the susceptibility of anionic charged molecules to self-aggregation, said methods comprising contacting said anionic charged molecules with a construct of formula I.

In further embodiments, the present invention provides methods for reducing the susceptibility of a double-stranded or hairpin nucleic acid to hydrolysis of the phosphodiester backbone, said methods comprising contacting said nucleic acid with a molecular entity of formula I.

In some embodiments, the present invention provides methods for preparing a construct of formula I comprising:
a) covalently attaching a first linker (L¹) to a first cationic arm (CA¹) to form L¹-CA¹ and a second linker (L²) to a second cationic arm (CA²) to form L²-CA²; and
b) covalently attaching L¹-CA¹ and L²-CA² to a cyclodextrin.

In related embodiments, the present invention provides methods for preparing a construct of formula I comprising:

a) covalently attaching linkers L¹ and L² to a cyclodextrin; and

b) covalently attaching cationic arms CA¹ and CA² to L¹ and L², respectively.

In some embodiments, each linker of the constructs may be independently selected from the group consisting of a covalent bond, a disulfide linkage, a protected disulfide linkage, an ether linkage, a thioether linkage, a sulfoxide linkage, an amine linkage, a hydrazone linkage, a sulfonamide linkage, an urea linkage, a sulfonate linkage, an ester linkage, an amide linkage, a carbamate linkage, a dithiocarbamate linkage, and the like, as well as combinations thereof. The linkage can be prepared in a variety of ways, e.g. by functional group conversion at one or more 6-positions of cyclodextrin (e.g. a thioether linkage, a sulfoxide linkage, an amine linkage, a sulfonamide linkage, a reverse ester linkage) and/or by linkage of 6-hydroxyl groups of cyclodextrin to appropriate linkers (e.g. an ester linkage, an ether linkage).

Exemplary constructs of formula I of the present invention can be chemically synthesized in a variety of ways. For example, according to the known procedure (see Tabushi et al., J. Am. Chem. Soc. 1984, 106, 5267-5270), beta-cyclodextrin can be selectively functionalized at the 6-positions of A,D-rings (Scheme 1). The A-D ring bridged compound 1 can then be converted to desired CD precursors suitable for cationic arms linkage, e.g. oligopeptides where the amino acid residues of the oligopeptide are capable of forming positive charges.

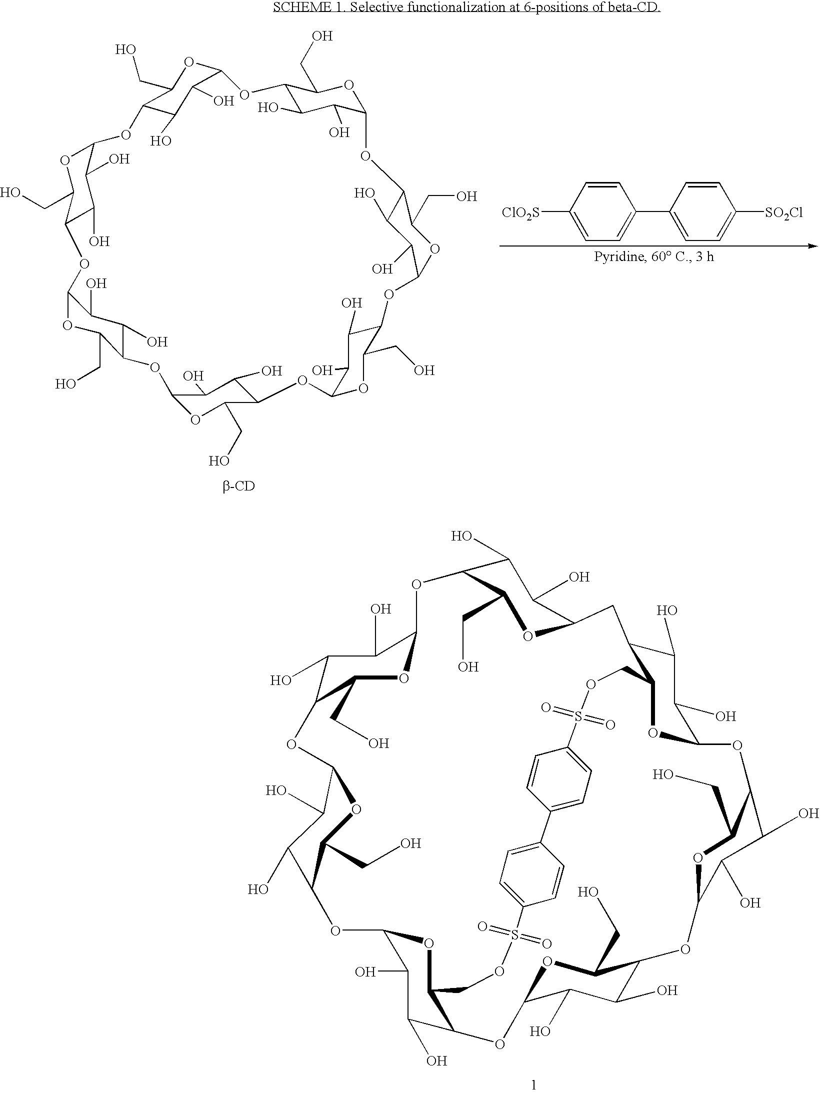

A skilled artisan could readily prepare different 6-position functionalized CDs from compound 1. For example, compound 1 can be converted to azido or iodo derivatives; the corresponding 6^{A},6^{D} di-azido or 6^{A},6^{D} di-iodo intermediates can then be converted to compounds 3 and 23 respectively (Scheme 2).

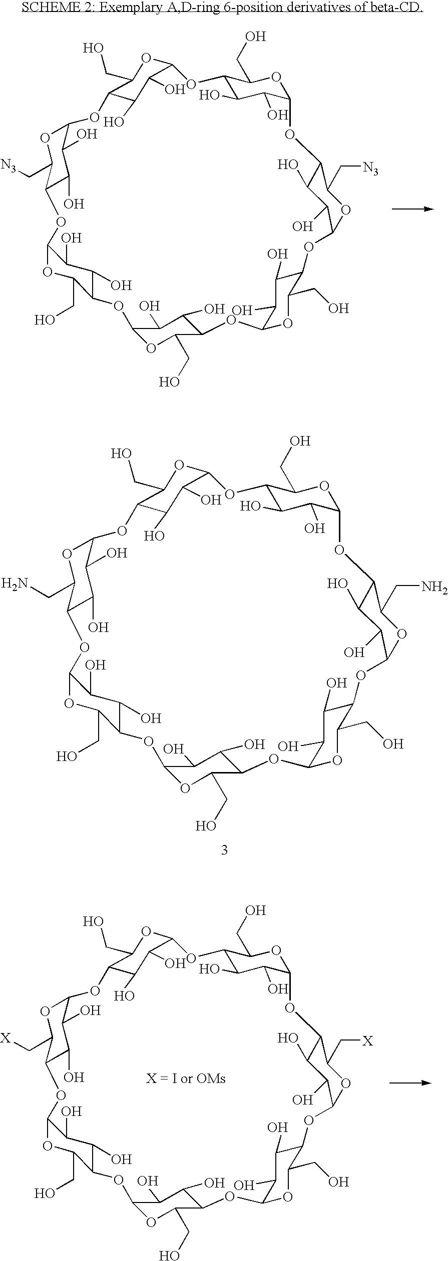

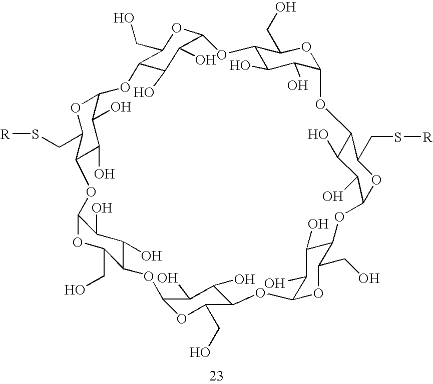

In other embodiments, the present invention provides compositions represented by formula II:

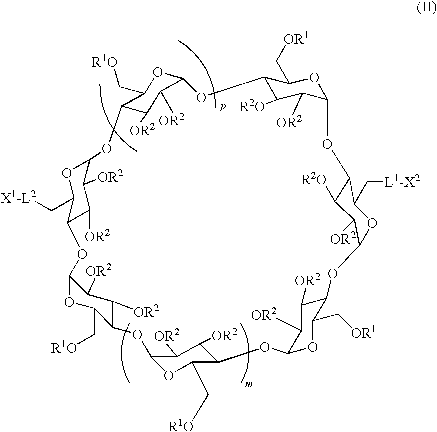

wherein
m is 0, 1 or 2;
p is 1 or 2, provided when p is 2, m is 1;
L¹ and L² are linkers independently selected from the group consisting of a covalent bond, a disulfide linkage, a protected disulfide linkage, an ether linkage, a thioether linkage, a sulfoxide linkage, a sulfonate linkage, an ester linkage, an amide linkage, a carbamate linkage, a dithiocarbamate linkage, an amine linkage, a hydrazone linkage, a sulfonamide linkage, an urea linkage, and combinations thereof;

R¹ is selected from the group consisting of hydrogen, alkyl, substituted alkyl, acyl, carbamoyl and silyl;

R² is selected from the group consisting of hydrogen, alkyl, substituted alkyl, and acyl;

X¹ and X² are displaceable functional groups; exemplary displaceable functional groups include azido, chloro, bromo, iodo, tosylate, substituted tosylate, triflate, mesylate, and the like; with the proviso that R¹ and R² are not the same; said ether linkage is not p-(allyloxy)phenyl ether linkage; and said amide linkage is not p-(allyloxy)benzoyl amide linkage.

As used herein, “displaceable functional group” is defined as an atom (or a group of atoms) that can be displaced under defined conditions such as SN₁, SN₂ or the like as stable species taking with it the bonding electrons. In some cases, leaving groups leave as anions, in others they leave as neutral molecules. The displaceable functional groups contemplated for use in the practice of the present invention may comprise azido, chloro, bromo, iodo, tosylate, substituted tosylate, triflate, mesylate or any other suitable leaving groups.

In some embodiments, the present invention provides methods for preparing compounds of formula II. The methods comprise reacting an optionally substituted 6-perbenzyl cyclodextrin (optionally substituted at one or more benzyl groups thereof) with a hydride reducing agent to produce a 6^{A},6^{D} or 6^{A},6^{E} dihydroxyl cyclodextrin. Preferably the optionally substituted 6-perbenzyl cyclodextrin is 6-per-(p-methoxybenzyl) cyclodextrin. The hydride reducing agent is preferably an aluminum hydride reducing agent; more preferably diisobutylaluminium hydride.

A presently preferred procedure to functionalize A,D-ring 6-positions involves selective reduction of protecting groups, e.g. optionally substituted benzyl, at the A-ring and D-ring of β-CD using a hydride reducing agent, e.g., Diisobutylaluminium hydride (DIBAH). The benzyl protecting groups may be substituted benzyl with electron donation groups such as p-methoxybenzyl (PMB) (see Scheme 3) or other suitable benzyl protecting groups at the A-ring and D-ring of β-CD. The differentiated 6-hydroxy groups can then be readily converted to azido or other functional groups by known procedures.

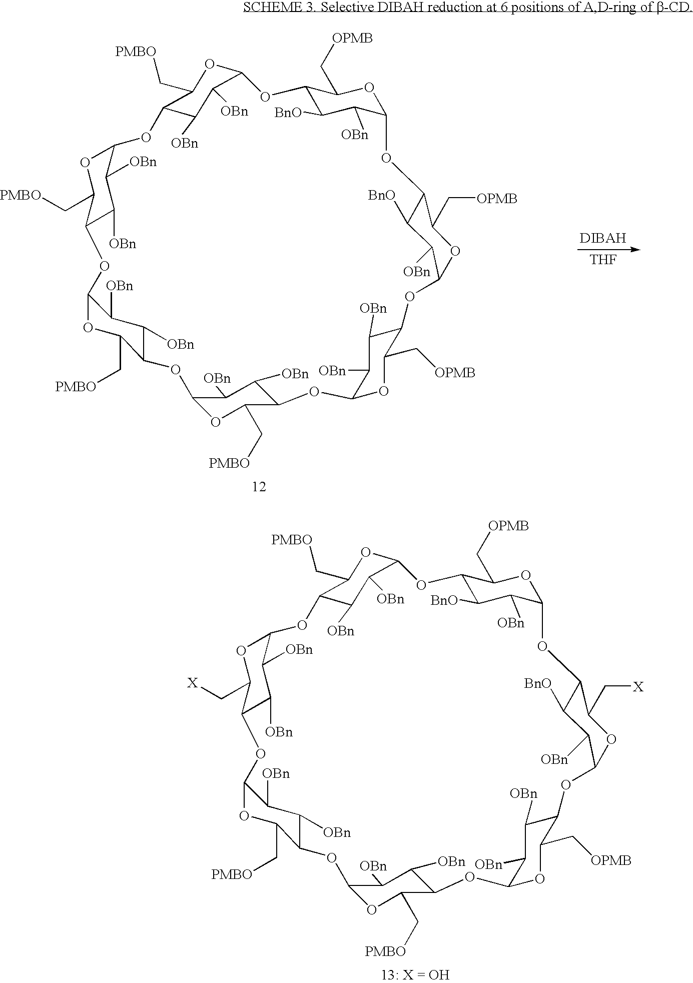

Examples of constructs prepared utilizing beta-CD functionalized 6-amine linkage (compounds 25) are illustrated in Scheme 4. Oligopeptides with positive charged functional groups can be readily prepared by standard peptide chemistry. Oligoamines can be readily prepared by known methods or are commercially available. The linkage between A⁶,D⁶-amine of CD and oligopeptides or oligoamines can readily be accomplished by amide bond formation.

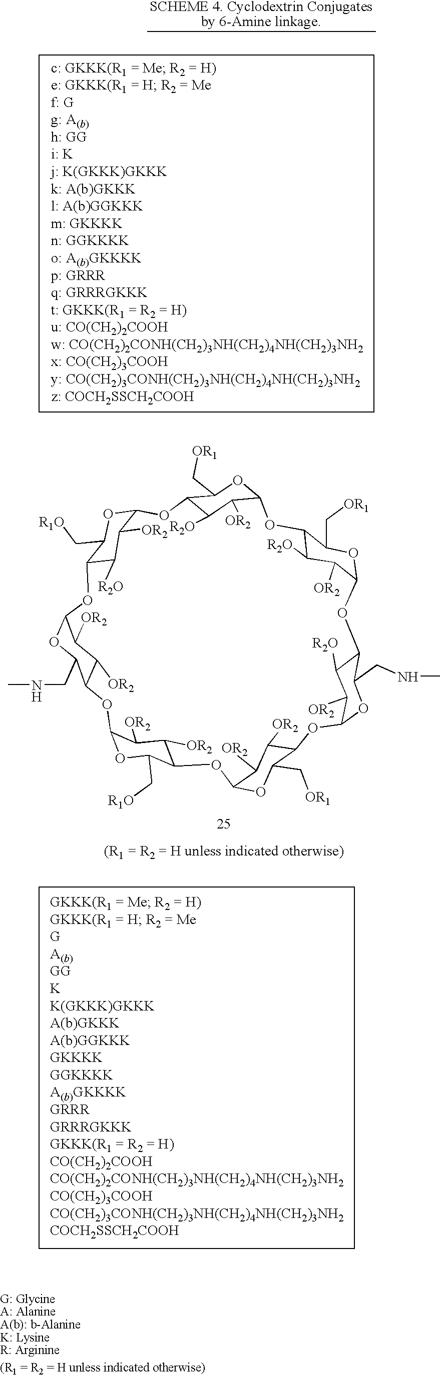

### Examples

The invention now being generally described, it will be more readily understood by reference to the following examples, which are included merely for purposes of illustration of certain aspects and embodiments of the present invention, and are not intended to limit the invention.

### Example 1

### Selective 6-OH Protection of a Beta-CD and Corresponding AD-Rings Homologation

The primary hydroxyl groups at A,D-rings can be readily protected by reaction of β-CD with biphenyl-4,4′-disulfonyl dichloride in the presence of amine base such as pyridine according to known procedures (Tabushi et al., J. Am. Chem. Soc. 1984, 106, 5267-5270). The desired compound 1 may be purified by suitable means, e.g. by reverse phase column chromatography. Amine moiety can be readily introduced at 6-position of A,D-rings. Compound 1 reacts with NaN₃ in DMF followed by triphenylphosphine (Ph₃P) reduction of azido groups to give desired compound 3.

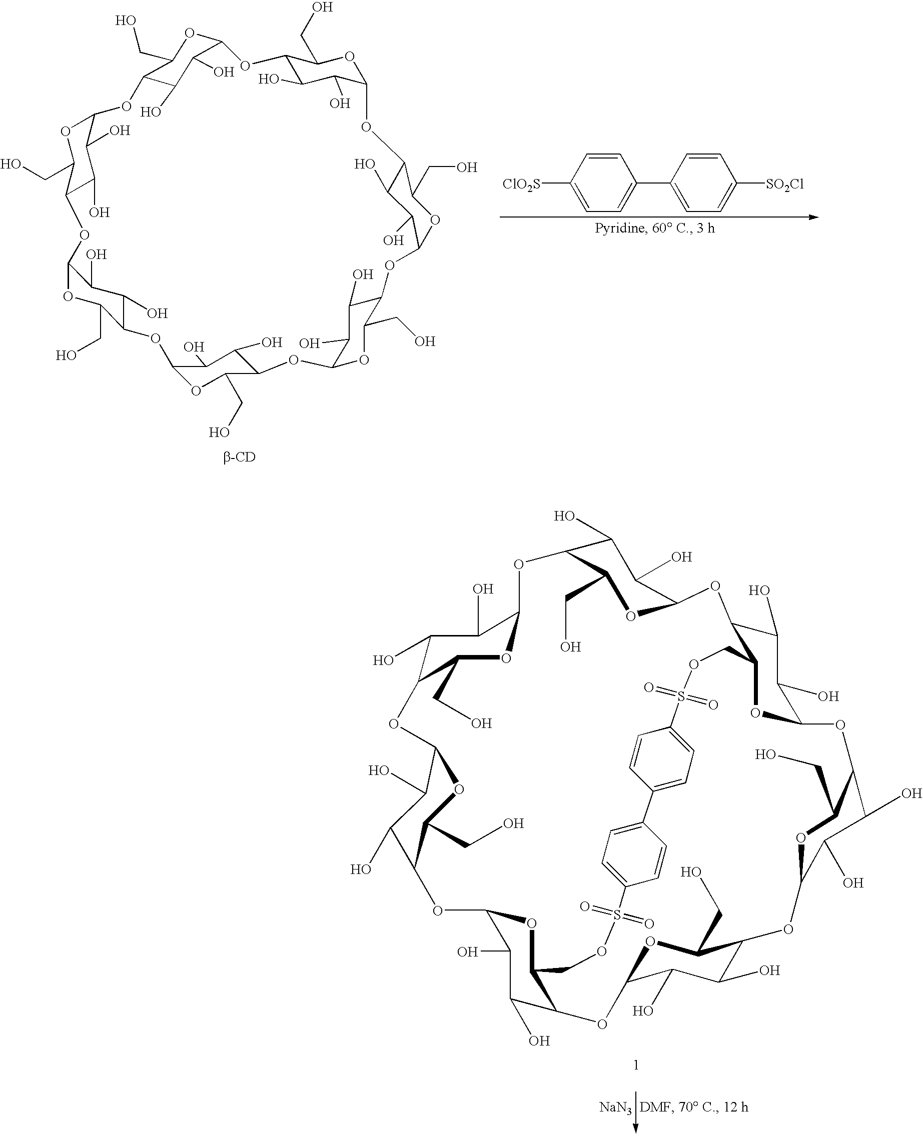

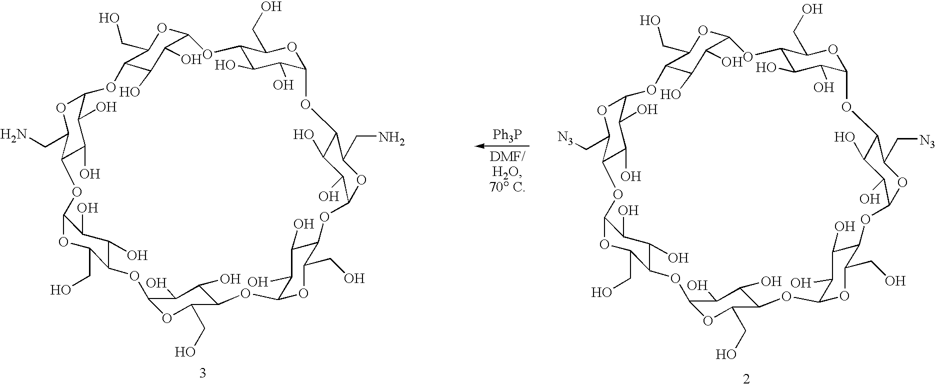

Alternatively, the procedure disclosed by Sinay et al. (Angew. Chem. Int, Ed. Engl., 2000, 39, 3610-3612) can be employed to selectively establish the 6^{A},6^{D}-ring functionality. Per-benzyl β-CD 4 is reduced at the 6^{A},6^{D}-ring to give 5 and subsequently to diamine 8 via mesylation (compound 6), azido conversion (compound 7) and azido reduction.

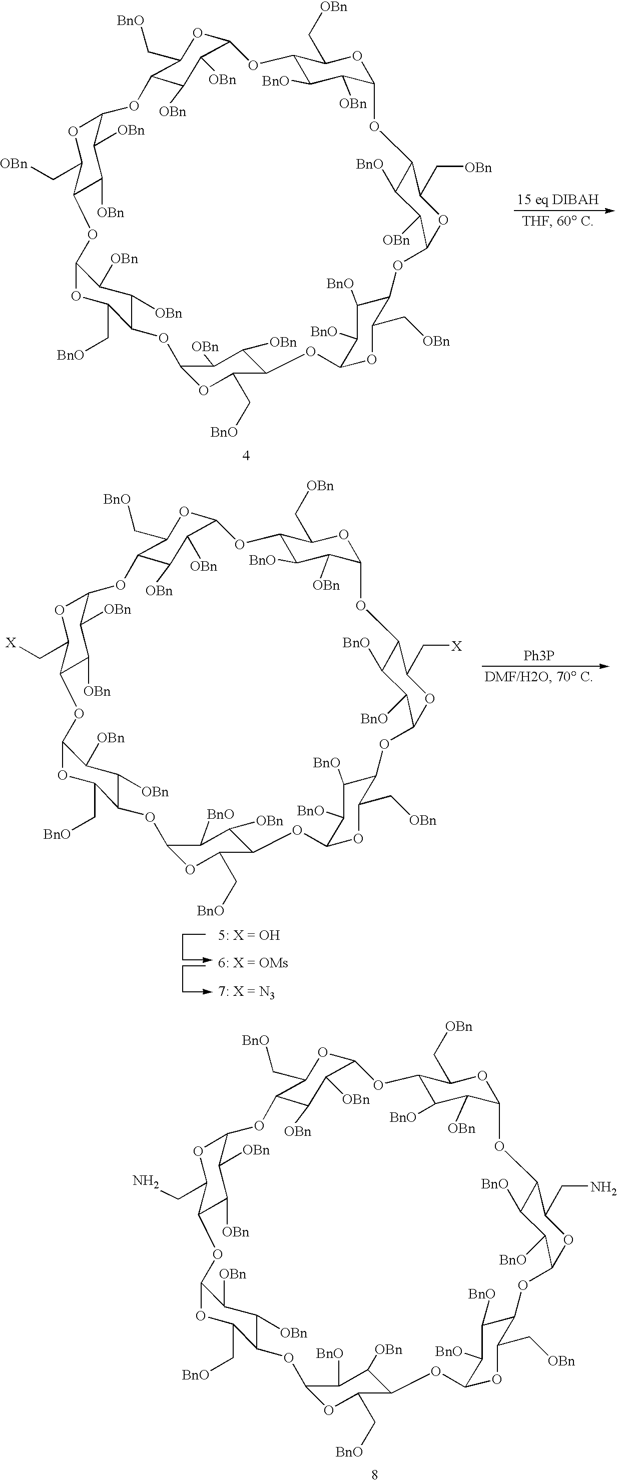

### Example 2

### Introduction of Substituents at 6^{A},6^{D} Ring Protected β-CD

Introduction of substituents at 6-positions of the A,D rings of β-CD can be achieved by discriminating the reactivity of the 2,3 positions versus the 6-position. All the 6-hydroxyl groups of β-CD are protected selectively with t-butyldimethytlsilylchloride (TBDMSCl) to give 9 followed by exhaustive benzylation of the remaining positions and deprotection of the 6-position resulting in 10. Alkylation of 10 with PMB-chloride affords 11 displaying two sets of orthogonal protecting groups. 11 is selectively reduced to 12 followed by two step functional group conversion to 14. Selective deprotection of 14 with acid gives 15 which can be selectively derivatized at the 6-position of B,C,E,F,and G rings by a skilled artisan to afford 16. Finally, reduction with trimethylphosphine (Me₃P) results in 17.

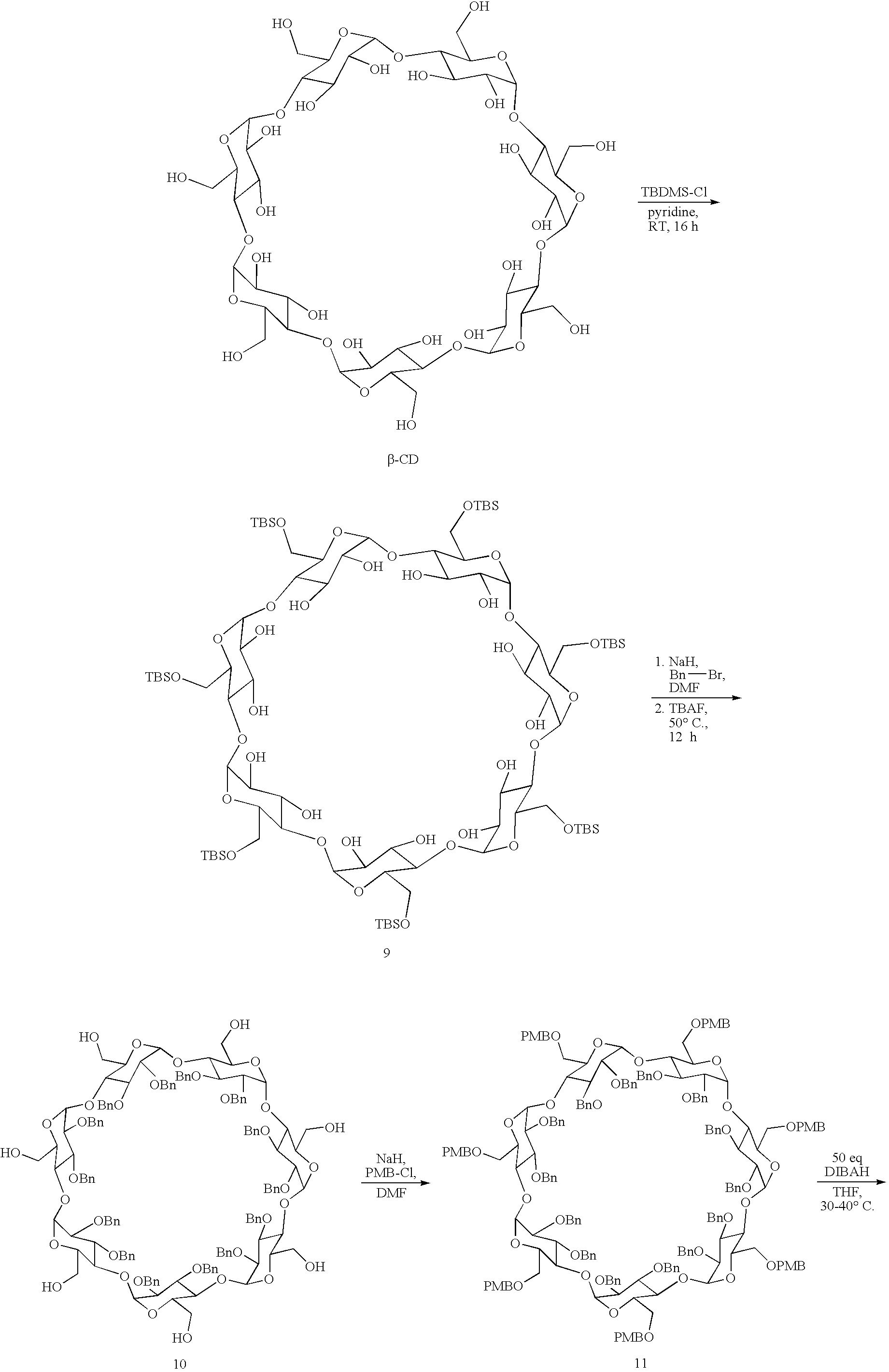

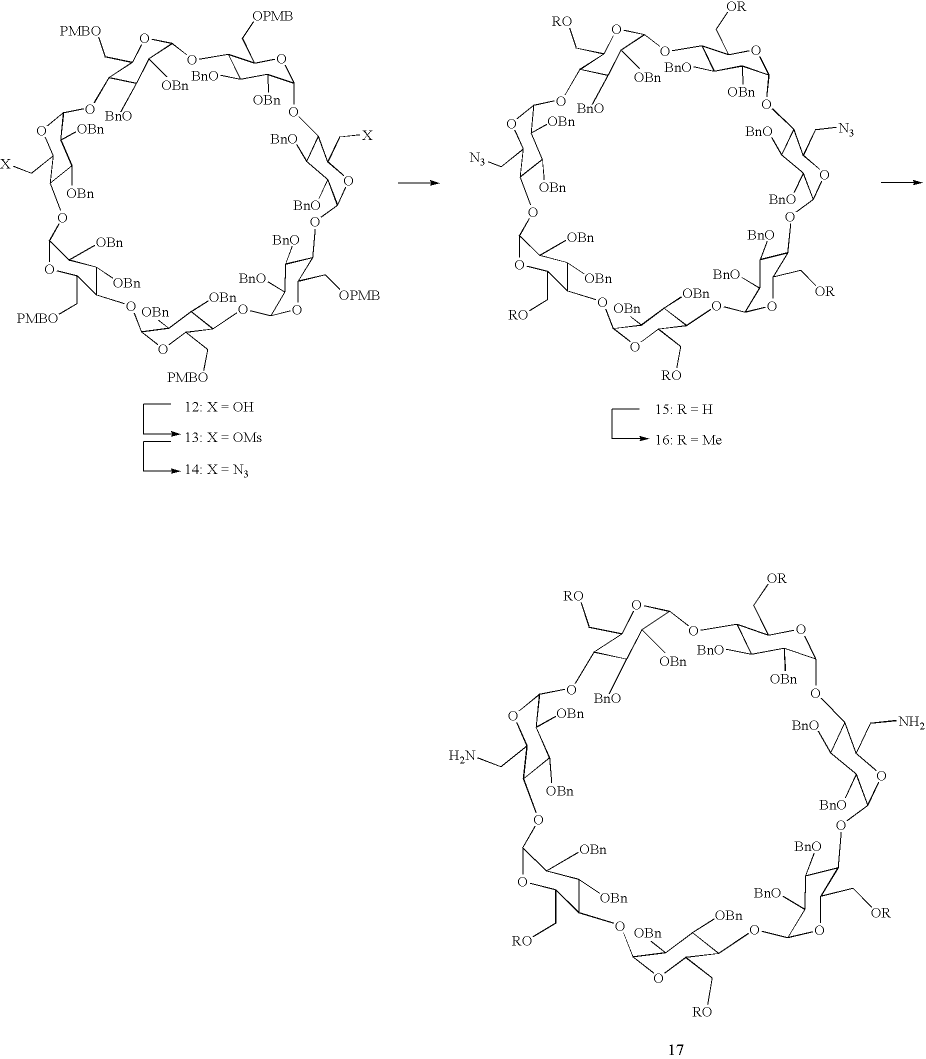

### Example 2-1

### Preparation of Compound 11

To a suspension of NaH (2.31 g, 57.83 mmols) in DMF (30 mL) at 0° C. under nitrogen was added a solution of 10 (9.90 g, 4.13 mmols) in DMF (50 mL) via syringe. The mixture was stirred at 0° C. for 10 minutes and at room temperature for 10 minutes. The mixture was re-cooled to 0° C. and PMBCl (7.85 mL, 57.83 mmols) was added drop wise via syringe. Stirring was continued and the mixture was warmed to room temperature overnight. The reaction mixture was cooled to 0° C., quenched with water slowly and concentrated under vacuum. The residue was dissolved in ethyl acetate and the organic phase was washed with 0.1 N aqueous HCl, followed by saturated aqueous NaHCO₃ and brine. The organic phase was then dried over anhydrous MgSO₄, filtered and concentrated under vacuum. The residue was purified by flash chromatography on silica gel employing hexanes and ethyl acetate as the eluting solvents to give 11.200 g (83%) of 11. ¹H NMR (300 MHz, CDCl₃): δ 3.41-3.50 (m, 14H), 3.60 (s, 21H), 3.7-5.2 (m, 77H), 6.70-7.41 (m, 98H).

### Example 2-2

### Preparation of Compound 12

The product 11 (6.70 g, 2.07 mmols) from above and molecular sieves (9 g, 4 \acute{Å}) were transferred into a flame-dried flask and kept under nitrogen. Dry toluene was added via syringe and the mixture equilibrated at 40° C. for 10 minutes. DIBAH (69 mL, 103.46 mmols) in toluene was added via syringe and the reaction was stirred for 45 minutes. The reaction mixture was cooled to −10° C. in an acetone/ice bath and carefully quenched with water. Ethyl acetate was added to the resulting suspension and then filtered through celite. The precipitate was further washed with hot ethyl acetate and the filtrates were combined. The combined filtrate was washed with brine, dried over anhydrous MgSO₄, filtered and concentrated under vacuum. The residue was purified by flash chromatography on silica gel employing hexanes and ethyl acetate as the eluting solvents to give 3.20 g (52%) of 12 as a white solid. ¹H NMR (300 MHz, CDCl₃): δ 3.41-5.60 (m, 104H), 6.70-7.70 (m, 90H).

### Example 2-3

### Preparation of Compound 13

A solution of 12 (2.00 g, 0.67 mmols) in dry pyridine (30 mL) under nitrogen was cooled to 0° C. and MsCl (0.26 mL, 3.34 mmols) was added via syringe. The reaction mixture was stirred to room temperature overnight and concentrated under vacuum at room temperature. The residue was taken up in ethyl acetate and washed with 0.1 N aqueous HCl, saturated aqueous NaHCO₃, brine, dried over anhydrous MgSO₄, filtered and concentrated under vacuum. The residue was purified by flash chromatography on silica gel employing hexanes and ethyl acetate as the eluting solvents to give 1.90 g (90%) of 13. ¹H NMR (300 MHz, CDCl₃): δ 2.60 (s, 6H), 3.20-3.50 (m, 8H), 3.65 (s, 15H), 3.65-5.40 (m, 79H), 6.60-7.60 (m, 90H).

### Example 2-4

### Preparation of Compound 14

NaN₃ (0.59 g, 9.03 mmols) was added to a solution of 13 (1.90 g, 0.60 mmols) in DMF (25 mL). The reaction mixture was stirred at 80° C. for 20 h, concentrated under vacuum, and treated with ethyl acetate. The ethyl acetate solution was washed with water, brine, dried over anhydrous MgSO₄, filtered and concentrated under vacuum to give 1.75 g (95%) of 14. ¹H NMR (300 MHz, CDCl₃): δ 3.30-3.70 (m, 20H) 3.70 (s, 15H), 3.75-4.2 (hs, 24H), 4.30-4.60 (m, 25H), 4.70 (bs, 9H), 4.90-5.40 (m, 9H), 6.70-7.70 (m, 90H).

### Example 2-5

### Preparation of Compound 15

10% TFA in dichloromethane (27 mL) was added to compound 14 (1.50 g, 0.49 mmols) at room temperature. The mixture was stirred at room temperature for 20 minutes and slowly added to saturated aqueous NaHCO₃ solution. The organic layer was separated and the aqueous phase extracted with dichloromethane (5 mL×5). The combined organic extracts were dried over anhydrous MgSO₄, filtered and concentrated under vacuum. The residue was purified by flash chromatography on silica gel employing 5% methanol in ethyl acetate as the eluting solvent to give 0.50 g (42%) of 15. ¹H NMR (300 MHz, CDCl₃): δ 2.90-4.25 (m, 47H), 4.30-5.50 (m, 35H), 7.20 (hs, 70H).

### Example 2-6

### Preparation of Compound 16

To a suspension of NaH (0.08 g, 2.04 mmols) in DMF (2 mL) at 0° C. and under nitrogen was added a mixture of 15 (0.40 g, 0.16 mmols) and MeI (0.13 mL, 2.04 mmols) in DMF (8 mL) via syringe. The mixture was stirred at 0° C. for 1 h and at room temperature for another 1 h. The mixture was re-cooled to 0° C., quenched with methanol and concentrated under vacuum. The residue was dissolved in dichloromethane, washed with water, aqueous Na₂S₂O₃, brine, dried over anhydrous MgSO₄, filtered and concentrated under vacuum. The residue was purified by flash chromatography on silica gel employing hexanes and ethyl acetate as the eluting solvents to give 0.244 g (59%) of 16. ¹H NMR (300 MHz, CDCl₃): δ 3.35 (s, 15H), 3.40-4.10 (m, 42H), 4.30-4.70 (m, 12H), 4.70-5.30 (m, 38H), 7.20 (bs, 70H).

### Example 2-7

### Preparation of Compound 17

To a solution of 16 (0.23 g, 0.09 mmols) in THF/0.1N NaOH; 9:1 (10 mL) at room temperature was added Me₃P (0.82 mL, 0.82 mmols). The resulting reaction mixture was stirred overnight and then concentrated under vacuum. The residue was taken up in ethyl acetate and washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.080 g (36%) of 17. ¹H NMR (300 MHz, CDCl₃): δ 2.90-3.20 (bs, 4H), 3.35 (s, 15H), 3.35-3.60 (m, 13H), 3.70-4.15 (m, 27H), 4.30-5.40 (m, 37H), 7.20 (bs, 70H).

### Example 3

### Introduction of Substituents at 2,3 Positions of the A,D Rings of β-CD

Selective silylation of the primary hydroxyl groups of 2 gives 18 followed by exhaustive derivatization of the 2,3-positions using excess reagent as shown below for the methylation of 18 to arrive at 19. Desilylation of 19 and subsequent reduction of 20 with Ph₃P gives diamine 21 ready for final assembly with cationic arms.

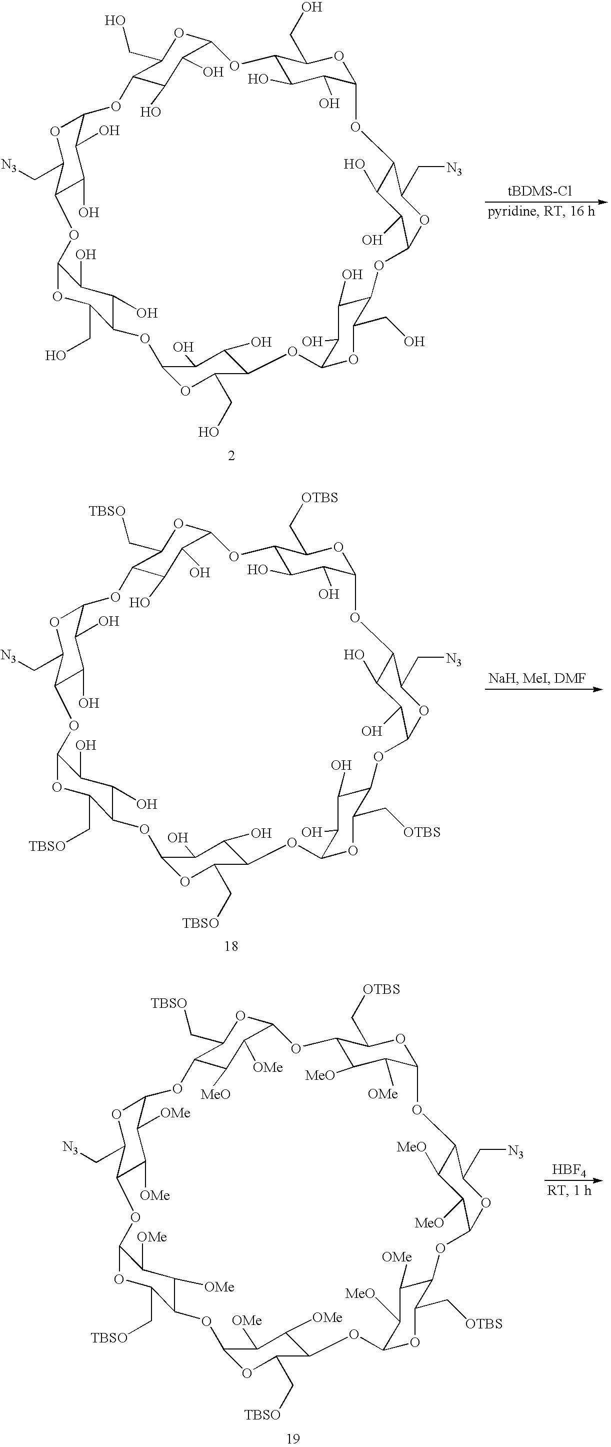

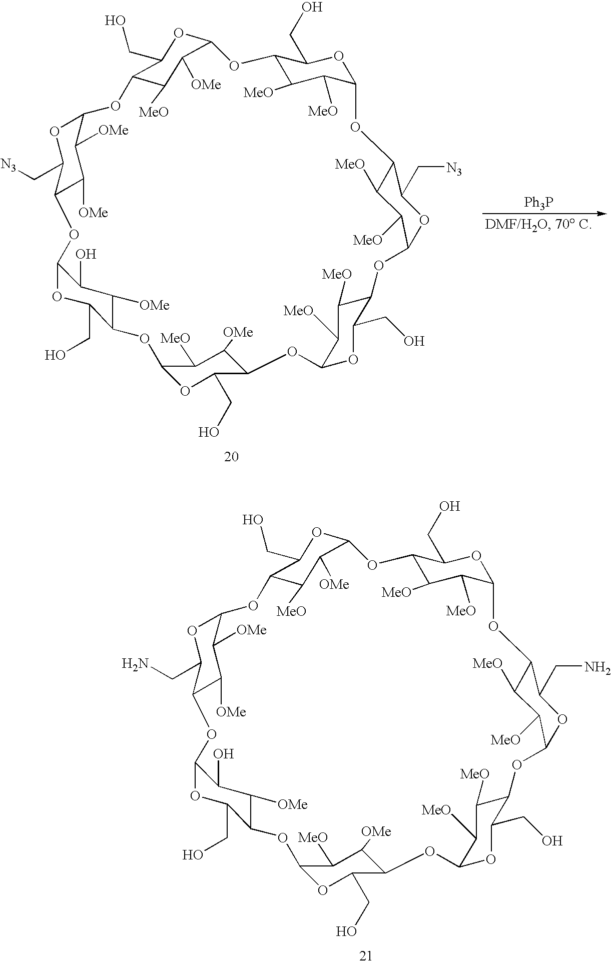

### Example 3-1

### Preparation of Compound 18

To solution of 95mg (0.080 mmol) of 2 in 1 ml absolute pyridine was added 84 mg (0.56 mmol) t-BDMSCl. The reaction mixture was stirred for 18 h at room temperature and then concentrated at vacuum. The semi crystalline residue was taken up in a few drops of methanol, re-precipitated from an excess of water and finally washed with ethyl acetate. Upon drying in vacuo 125 mg (89%) colorless precipitate was obtained. ¹H NMR (300 MHz, CDCl₃): δ −0.1-0.0(30H), δ 0.95-1.10 (45H), 3.25-4.05 (m, 42H), 4.8-4.95 (m, 7H).

### Example 3-2

### Preparation of Compound 19

To a suspension of NaH (100 mg, 2.5 mmols) in DMF (3 mL) at 0° C. under nitrogen was added a solution of 18 (120 mg, 0.068 mmols) in DMF (2 mL) via syringe. The mixture was stirred at 0° C. for 10 minutes and at room temperature for 10 minutes. The mixture was re-cooled to 0° C. and methyliodide (0.125 ml, 2.0 mmols) was added drop wise via syringe. Stirring was continued and the mixture was warmed to room temperature overnight. After cooling the reaction mixture to 0° C. it was slowly quenched with water and concentrated under vacuum. The residue was dissolved in ethyl acetate and the organic phase was washed with 0.1 N aqueous HCl, followed by saturated aqueous NaHCO₃ and brine. Drying over anhydrous MgSO₄, followed by filtration and concentration under vacuum gave an oily residue which was purified by flash chromatography on silica gel employing hexanes and ethyl acetate as the eluting solvents to give 75 mg (57%) of 19. ¹H NMR (300 MHz, CDCl₃): δ 0.0(s, 30H), δ 0.82 (s, 45H), 2.95-3.18 (m, 7H), 3.3-4.2 (m, 84H) 5.02-5.25 (m, 7H).

### Example 3-3

### Preparation of Compound 20

HBF₄ was added via syringe to compound 19 (0.42 g, 0.21 mmols) in acetonitrile (13 mL) solution in a polyethylene container at room temperature. The mixture was stirred for 1 h at room temperature, quenched with saturated aqueous NaHCO₃ solution and extracted several times with dichloromethane. The extracts were combined, washed with brine, dried over anhydrous MgSO₄, filtered and concentrated under vacuum to give 0.230 g (77%) of 20. ¹H NMR (300 MHz, CDCl₃): δ 3.20 (bs, 9H), 3.30-4.00 (78H), 5.1 (m, 9H).

### Example 3-4

### Preparation of Compound 21

To 20 (0.20 g, 0.15 mmols) dissolved in DMF (5 mL) and H₂O (0.5 mL) was added prewashed polymer-bond Ph₃P (0.29 g, 0.88 mmols; 3 mmols/g loading). The mixture was stirred at 60° C. overnight, the resin filtered-off, and the filtrate was concentrated under vacuum. The residue was dissolved again in DMF (5 mL) and H₂O (0.5 mL) and 10 eq. of polymer-bond Ph₃P (0.48 g, 1.46 mmols; 3 mmols/g loading) was added. The mixture was heated at 70° C. overnight, filtered off resin and the filtrate concentrated under vacuum. The residue was purified by flash chromatography on silica gel employing 2% NH₄OH/20% methanol in dichloromethane as the eluting solvent to give 0.100 g (51%) of 21. ¹H NMR (300 MHz, CDCl₃): δ 3.00-4.00 (91H), 5.1 (m, 9H).

### Example 4

### Preparation of 6^{A},6^{D} Ring β-CD with Mercapto Linker

The 6^{A},6^{D} di-iodo β-CD can be prepared according to known procedures (Hwang et al., Bioconjugate Chem. 2001, 12, 280). Compound 22 can be prepared by reaction of 1 with KI in DMF at 80° C. for 2 hours. Compound 22 is then readily available for derivatization via nucleophilic substitution to give thioether 23.

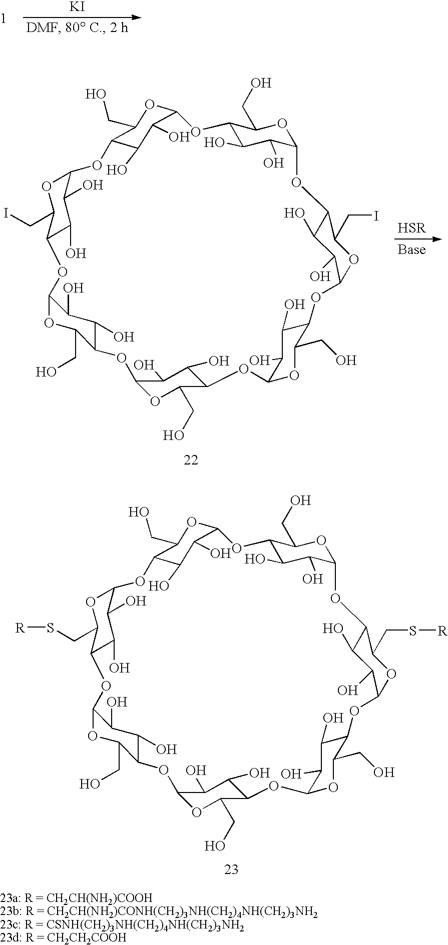

### Example 4-1

### Preparation of Compound 23a

KOH (0.1 g, 1.5 mmol, 10 eq) was added to a solution of compound 22 (0.2 g, 0.15 mmol) in DMF (2 ml). After being purged with nitrogen, Boc-Cys (89 mg, 0.44 mmol, 3.3 eq) was added to the reaction mixture and then purged again with nitrogen. The resulting reaction mixture was stirred at room temperature for 24 h. The solvent was removed under reduced pressure and the residue was washed with water, ethyl acetate and then was dried under vacuum to yield product 23a as a white solid (0.22, 80%). ¹H-NMR (300 MHz, D₂O) δ 1.25-1.5 (s, 18H), 3.2-4.1 (br, 48H), 4.85-5.00 (s, 7H).

### Example 4-2

### Preparation of Compound 23b

Compound 23b was synthesized employing similar procedures for the formation of amide bond and the subsequent deprotection of Boc group using compound 23a (0.1 g, 0.065 mmol) and NH₂(CH₂)₃N(Boc)(CH₂)₄N(Boc)(CH₂)₃NH(Boc) (0.068 g, 0.135 mmol, 2 eq) to yield product 23b (70 mg, 63%). ¹H-NMR (300 MHz, D₂O) δ 1.00-2.0 (m, 16H), 2.8-4.0 (m, 72H), 5.00 (s, 7H).

### Example 4-3

### Preparation of Compound 23c

To a solution of compound 22 (0.1 g, 0.075 mmol) and NH₂(CH₂)₃N(Boc)(CH₂)₄N(Boc)(CH₂)₃₋NH(Boc) (90 mg, 0.18 mmol, 2.4 eq) were added K₃PO₄ (165 mg, 0.72 mmol, 4.8 eq) and carbon disulfide (43 μl, 0.72 mmol, 4.8 eq). The resulting mixture was stirred at ambient temperature for 24 h. The solvent was evaporated and the residue was dissolved in water and then washed with ethyl acetate. The aqueous solution was evaporated to dryness and then slurried with water to provide a solid compound after drying under reduced pressure. The dried compound was dissolved in 75% TFA/CH₂Cl₂ and stirred for 3 h. The solvent was evaporated under reduced pressure to yield product 23c as a pale yellow solid (80 mg, 46%). ¹H-NMR (300 MHz, D₂O) δ 1.00-2.0 (m, 16H), 3.0-4.2 (m, 66H), 5.00 (s, 7H).

### Example 4-4

### Preparation of Compound 23d

To a solution of 22 (0.200 g, 0.147 mmols) in DMF (4 mL) was added 3-mercaptopropionic acid (0.128 mL, 1.476 mmols) and NEt₃ (0.103 mL, 0.738 mmols) at room temperature and under nitrogen. The mixture was heated at 60° C. overnight with stirring. The mixture was concentrated to near dryness and acetone added. The precipitate formed was further washed with acetone, 5% water in acetone and dried under vacuum at 60° C. for 5 h to give 23d (0.165 g, 85%) as an off-white solid. ¹H NMR (300 MHz, DMSO-d₆): δ 2.55-3.10 (m, 7H), 3.50-4.10 (bs, 35H), 4.10-4.70 (m, 6), 4.70-5.20 (m, 10H), 5.40-6.30 (m, 18H).

### Example 5

### Preparation of 6^{A},6^{D} Ring-Derivatized CD with Di-Thioether Linker

Reaction of 6^{A},6^{D}-diamino β-CD with dithioether containing compounds will lead to β-CD substituted with di-thioether linkers. For example, starting with compound 3, dithiodiglycolic acid will give compounds with dithioether bridges such as 24. Compound 24 can then be selectively coupled to the amino terminus of an oligopeptide. A skilled artisan also can prepare other derivatives following procedures known in the art.

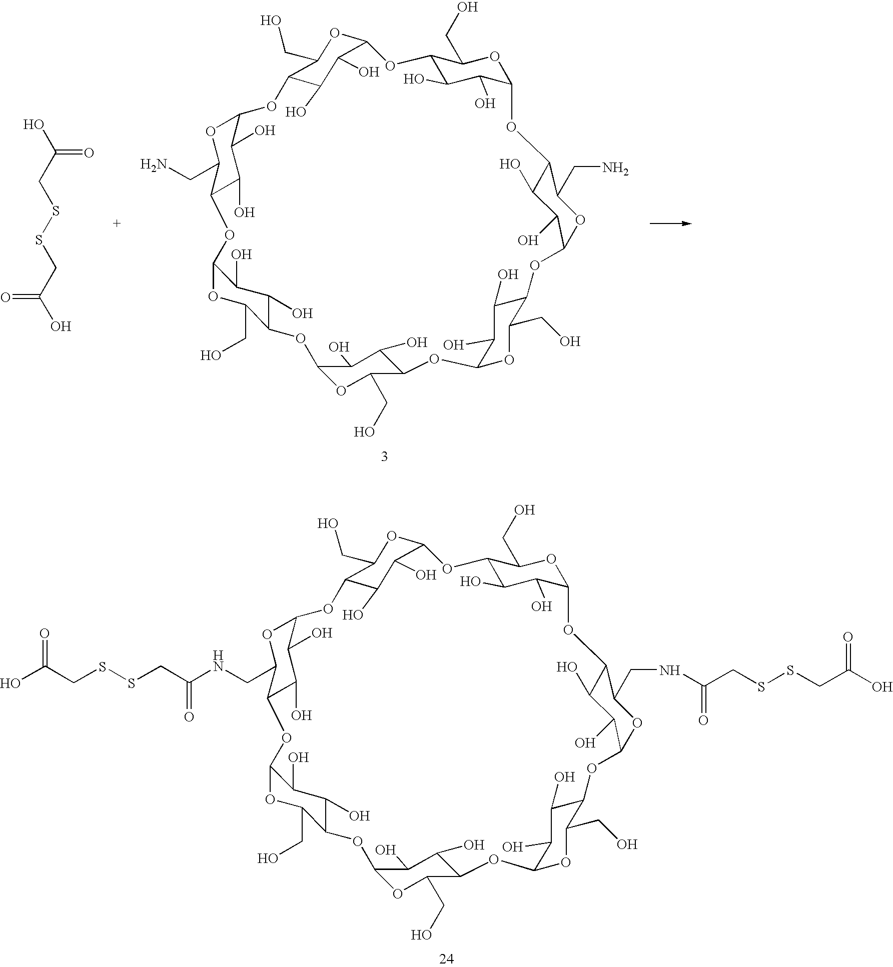

### Example 6

### Preparation of Oligopeptides

Oligopeptides such as oligolysine, oligoarginine or any suitable oligopeptide with amine moiety can be prepared via standard solid phase peptide synthesis. Examples used here may include any oligolysine up to twelve-mer.

### Example 7

### Synthesis of Oligopeptide-Cyclodextrin Conjugates 25

Reaction of compounds 3, 8, 17, and 21 with the C-terminus of an oligopeptide affords compounds 25. Upon removal of protecting groups such as Boc or Cbz, the desired construct suitable to complex with siRNA can be readily prepared.

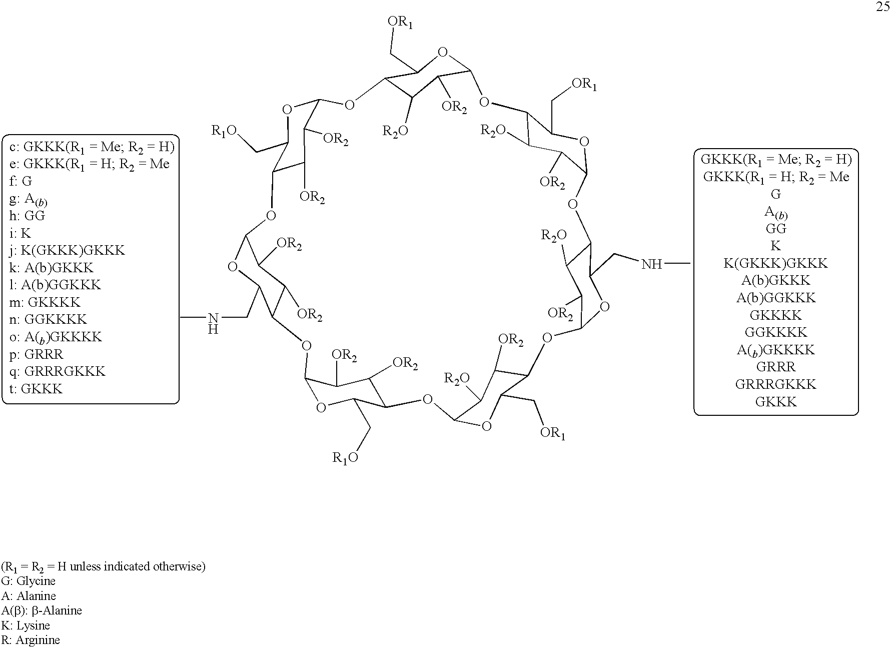

### Example 7-1

### General Procedure for the Formation of Peptide Bond

To a solution of cyclodextrin compounds with free amino groups (1 eq) and C-terminus oligopeptide or simple amino acid with all amino groups protected as t-butyl carbamate (Boc) or 9-fluorenylmethyl carbamate (Fmoc) (2.2 eq) in anhydrous DMF in an ice bath was added hydroxybenzotriazole (HOBt) (2.2 eq). The resulting solution was stirred at 0° C. for 30 min. Dicyclohexylcarbodiimide (DCC) (2.2 eq) was then added. The mixture was stirred at 0° C. to room temperature until the reaction was complete (monitored by HPLC). The precipitated dicyclohexylurea (DCU) was filtered off and the filtrate was concentrated under reduced pressure. The residue was slurried with ethyl acetate and then filtered or decanted. The solid containing the desired compound and DCU was used in the next step without further purification.

### Example 7-2

### General Procedure for the Deprotection of Boc Protected Amino Group

The Boc protected amino compound was dissolved in trifluoroacetic acid (TFA) and dichloromethane (25%). The resulting solution was stirred at room temperature for 0.5-3 hours. The solvent was evaporated under reduced pressure and the residue was dissolved in water. The undissolved DCU was filtered off and the filtrate was evaporated under reduced pressure to give the desired compound.

### Example 7-3

### General Procedure for the Deprotection of Fmoc Protected Amino Group

The Fmoc protected amino compound was dissolved in DMF and the piperidine was added. The resulting solution was stirred at room temperature for several hours until the protecting group was completely removed (monitored by HPLC). The solvent was evaporated under reduced pressure and the residue was dissolved in water, filtered and washed with ethylacetate. The aqueous phase was evaporated to dryness to give the desired product.

### Example 7-4

### Preparation of Tetramer Peptide CD Conjugate 25a

To a solution of 17 (0.08 g, 0.03 mmols) and Boc-Lys(Boc)-Lys(Boc)-Lys(Boc)-Gly-OH (0.07 g, 0.08 mmols) in DMF was added HOBt (0.01 g, 0.08 mmols) and DCC (0.02 g, 0.08 mmols) at room temperature. The mixture was stirred at room temperature overnight and an additional DCC (10 mg) and HOBt (8 mg) was added. The reaction was further stirred at room temperature overnight, concentrated to near dryness under vacuum and the residue was treated with ethyl acetate. The organic phase was washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.112 g (36%) of 25a. ¹H NMR (300 MHz, CDCl₃): δ 1.40 (s, 72H), 1.55-6.10 (m, 218H), 7.20 (m, 90H).

### Example 7-5

### Preparation of Tetramer Peptide CD Conjugate 25b

To the above compound 25a (0.11 g, 0.03 mmols) in THF (10 mL) was added 10% Pd/C and palladium black (0.03 g). The reaction mixture was evacuated and flushed three times with a hydrogen filled balloon before stirring was continued for 48 h. The reaction mixture was filtered through celite and the catalyst (10% Pd/C and palladium black) was washed with THF. The filtrate was concentrated, treated with acetone and the precipitate washed several times with acetone. The precipitate was then dried under vacuum at 60° C. overnight to give 0.063 g (84%) of 25b. MS m/z Calcd for (M+H)⁺C₁₂₇H₂₂₄N₁₆O₅₇: 2887.21; Found: 2888.00.

### Example 7-6

### Preparation of Tetramer Peptide CD Conjugate 25c

To compound 25b (0.04 g, 0.015 mmols) was added 75% TFA in dichloromethane (3 mL) and the resulting reaction mixture was stirred at room temperature for 2.5 h. The mixture was concentrated under vacuum, triturated with cyclohexane and the precipitate collected by filtration. The precipitate was then dried under vacuum at 60° C. overnight to give 0.047 g 100%) of 25c. MS m/z Calcd for (M+H)⁺C₈₇H₁₆₀N₁₆O₄₁: 2086.28; Found: 2087.40.

### Example 7-7

### Preparation of Tetramer Peptide CD Conjugate 25d

To a solution of 21 (0.05 g, 0.04 mmols) and Boc-Lys(Boc)-Lys(Boc)-Lys(Boc)-Gly-OH (0.08 g, 0.09 mmols) in DMF was added HOBt (0.01 g, 0.09 mmols) and DCC (0.02 g, 0.08 mmols) at room temperature. The mixture was stirred at room temperature overnight under nitrogen, concentrated to near dryness under vacuum and the residue treated with ethyl acetate. The organic phase was washed with saturated aqueous NaHCO₃, brine, dried over anhydrous MgSO₄, filtered and concentrated under vacuum. The residue was purified by flash chromatography on silica gel employing 10% methanol in dichloromethane as the eluting solvent to give 0.025 g (22%) of 25d. ¹H NMR (300 MHz, CDCl₃): δ 0.60-0.90 (m, 5H), 1.10-1.50 (bs, 117H), 1.50-1.70 (bs, 8H), 1.97 (s, 5H), 2.20 (bs, 9H), 2.90-3.25 (m, 24H), 3.30-3.65 (m, 59H), 3.65-4.00 (m, 4H), 4.80-5.20 (m, 11H).

### Example 7-8

### Preparation of Tetramer Peptide CD Conjugate 25e

To compound 25d (0.02 g, 0.001 mmols) was added 75% TFA in dichloromethane (5 mL) and the resulting reaction mixture was stirred at room temperature for 1.5 h. The mixture was concentrated under vacuum, triturated with cyclohexane and the precipitate collected by filtration. The precipitate was then dried under vacuum at 50° C. for 48 h to give 0.025 g 100%) of 25e. MS m/z Calcd for (M+H)⁺C₉₆H₁₇₈N₁₆O₄₁: 2212.52; Found: 2213.50.

### Example 7-9

### Preparation of 6^{A},6^{D}-dideoxy-6^{A},6^{D}-di(Gly)amino-β-cyclodextrin (25f)

Compound 25f was synthesized as described in the general procedures for the formation of CD-peptide bond and the subsequent deprotection of Fmoc group using 6^{A},6^{D}-dideoxy-6^{A},6^{D}-diamino-β-cyclodextrin (3) (0.4 g, 0.35 mmol) and Fmoc-glycine (0.228 g, 0.77 mmol, 2.2 eq) to yield product 25f (0.35 g, 80%) as a pale yellow solid. ¹H-NMR (300 MHz, D₂O) δ 3.0-4.0 (m, 46H), 5.08 (s, 7H).

### Example 7-10

### Preparation of 6^{A},6^{D}-dideoxy-6^{A},6^{D}-di(β-Ala)amino-β-cyclodextrin (25g)

Compound 25 g was synthesized as described in the general procedures for the formation of CD-peptide bond and the subsequent deprotection of Fmoc group using compound 3 (0.4 g, 0.35 mmol) and Fmoc-β-alanine (0.24 g, 0.77 mmol, 2.2 eq) to yield product 25 g (0.12 g, 27%) as a off-white solid. ¹H-NMR (300 MHz, DMSO-d₆) δ 3.0-4.3 (m, 75H), 4.80-4.90 (m, 7H).

### Example 7-11

### Preparation of 6^{A},6^{D}-dideoxy-6^{A},6^{D}-di(Gly-Gly)amino-β-cyclodextrin (25h)

Compound 25h was synthesized as described in the general procedures for the formation of CD-peptide bond and the subsequent deprotection of Fmoc group using compound 25f (0.5 g, 0.39 mmol) and Fmoc-glycine (0.260 g, 0.87 mmol, 2.2 eq) to yield product 25h (0.2 g, 37%) as pale yellow solid. ¹H-NMR (300 MHz, D₂O) δ 3.0-4.0 (m, 50H), 4.99 (s, 7H); MS m/z Calcd. for C₅₀H₈₄N₆O₃₇ 1360.49, Found 1361.7.

### Example 7-12

### Preparation of 6^{A},6^{D}-dideoxy-6^{A},6^{D}-di(Lys)amino-β-cyclodextrin (25i)

Compound 25i was synthesized as described in the general procedures for the formation of the CD-peptide and the subsequent deprotection of Boc group using compounds 3 (0.1 g, 0.085 mmol) and Boc-Lys(Boc)-OH (0.077 g, 0.185 mmol, 2.2 eq) to yield product 25i (0.04 g, 34%) as a pale yellow solid. ¹H-NMR (300 MHz, D₂O) 6 1.48-1.65 (m, 12H), 2.87 (t, 4H), 3.26-3.95(m, 44H), 4.95(s, 7H).

### Example 7-13

### Preparation of 6^{A},6^{D}-dideoxy-6^{A},6^{D}-di[Lys-(Gly-Lys-Lys-Lys-NH₂)-Gly-Lys-Lys-Lys-NH₂]amino-β-cyclodextrin (25j)

Compound 25j was synthesized as described in the general procedures for the formation of the CD-peptide and the subsequent deprotection of Boc group using compound 25i (0.020 g, 0.014 mmol), Boc-Lys(Boc)-Lys(Boc)-Lys(Boc)-Gly-OH (0.054 g, 0.063 mmol, 4.5 eq) and compound 3 to yield 50 mg product 25j (50 mg, 71%) as a pale yellow oil. ¹H-NMR (300 MHz, CD₃OD) δ 1.05-2.00 (m, 84H), 2.75-3.00 (m, 28H), 3.26-3.953, 30-4.40(m, 64H), 4.95(s, 7H, merged with H₂O peak).

### Example 7-14

### Preparation of 6^{A},6^{D}-dideoxy-6^{A},6^{D}-di(Ala(_{β})-Gly-Lys-Lys-Lys-NH₂)amino-β-cyclodextrin (25k)

Compound 25k was synthesized as described in the general procedures for the formation of CD-peptide and the subsequent deprotection of Boc group using compound 25g (0.020 g, 0.0156 mmol) and Boc-Lys(Boc)-Lys(Boc)-Lys(Boc)-Gly-OH (0.030 g, 0.0348 mmol, 2.2 eq) to yield product 25k (40 mg, 81%) as a pale yellow solid ¹H-NMR (300 MHz, D₂O) δ 1.05-2.00 (m, 36H), 2.30-4.2 (m, 72H), 4.95(s, 7H); MS m/z Calcd for C₈₈H₁₆₀N₁₈O₄₃ 2158.3, Found 1080.41 ([M+2]⁺⁺/2).

### Example 7-15

### Preparation of 6^{A},6^{D}-dideoxy-6^{A},6^{D}-di(Ala(_{β})-Gly-Gly-Lys-Lys-Lys-NH₂)amino-β-cyclodextrin (25l)

Compound 25l was synthesized as described in the general procedures for the formation of CD-peptide bond and the subsequent deprotection of Boc group using compound 25 g (0.020 g, 0.0156 mmol) and Boc-Lys(Boc)-Lys(Boc)-Lys(Boc)-Gly-Gly-OH (0.030 g, 0.0348 mmol, 2.2 eq) to yield product 25l (17 mg, 53%) as an off white solid. ¹H-NMR (300 MHz, D₂O) δ 1.05-2.00 (m, 36H), 2.30-4.2 (m, 76H), 4.95(s, 7H); MS m/z Calcd for C₉₂H₁₆₆N₂₀O₄₅ 2272.4 Found 1137.23 ([M+2]⁺⁺/2).

### Example 7-16

### Preparation of 6^{A},6^{D}-dideoxy-6^{A},6^{D}-di(Gly-Lys-Lys-Lys-Lys-NH₂)amino-β-cyclodextrin (25m)

Compound 25m was synthesized as described in the general procedures for the formation of CD-peptide and the subsequent deprotection of Boc group using compound 3 (0.020 g, 0.0175 mmol) and Boc-Lys(Boc)-Lys(Boc)-Lys(Boc)-Lys(Boc)-Gly-OH (0.042 g, 0.0386 mmol, 2.2 eq) to yield product 25m (26 mg, 43%) as a white solid. ¹H-NMR (300 MHz, D₂O) δ 1.25-2.00 (m, 36H), 2.70-4.2 (m, 68H), 4.95(s, 7H); MS m/z Calcd for C₉₂H₁₆₆N₂₀O₄₅ 2158.3, Found 1137.23 ([M+2]⁺⁺/2).

### Example 7-17

### Preparation of 6^{A},6^{D}-dideoxy-6^{A},6^{D}-di(Gly-Gly-Lys-Lys-Lys-Lys-NH₂)amino-β-cyclodextrin (25n)

Compound 25n was synthesized as described in the general procedures for the formation of CD-peptide and the subsequent deprotection of Boc group using compound 25f (0.040 g, 0.032 mmol) and Boc-Lys(Boc)-Lys(Boc)-Lys(Boc)-Lys(Boc)-Gly-OH (0.076 g, 0.070 mmol, 2.2 eq) to yield product 25n (15 mg, 13%) as a off white solid. ¹H-NMR (300 MHz, D₂O) δ 1.25-2.00 (m, 48H), 2.80-4.2 (m, 74H), 4.95(s, 7H).

### Example 7-18

### Preparation of 6^{A},6^{D}-dideoxy-6^{A},6^{D}-di(Ala(p)-Gly-Lys-Lys-Lys-Lys-NH₂)amino-β-cyclodextrin (25o)

Compound 25o was synthesized as described in the general procedures for the formation of CD-peptide bond and the subsequent deprotection of Boc group using compound 25 g (0.020 g, 0.016 mmol) and Boc-Lys(Boc)-Lys(Boc)-Lys(Boc)-Lys(Boc)-Gly-OH (0.038 g, 0.035 mmol, 2.2 eq) to yield product 25o (14 mg, 25%) as a off white solid. ¹H-NMR (300 MHz, D₂O) δ 1.25-2.00 (m, 48H), 2.30-4.2 (m, 78H), 4.95(s, 7H); MS m/z Calcd for C₁₀₀H₁₈₄N₂₂O₄₅ 2414.65, Found 1208.33 ([M+2]⁺⁺/2).

### Example 7-19

### Preparation 6^{A},6^{D}-dideoxy-6^{A},6^{D}-di(Gly-Arg-Arg-Arg-NH₂)amino-β-cyclodextrin (25p)

Compound 25p was synthesized as described in the general procedures for the formation of CD-peptide bond and the subsequent deprotection of Fmoc group using compound 3 (0.030 g, 0.026 mmol) and Fmoc-Arg-Arg-Arg-Gly-OH (0.046 g, 0.06 mmol, 2.2 eq) to yield product 25p (50 mg, 88%) as an oil. ¹H-NMR (300 MHz, D₂O) δ 1.40-2.00 (m, 24H), 300-4.25 (m, 64H), 4.95(s, 7H); MS m/z Calcd for C₈₂H₁₅₀N₂₈O₄₁ 2184.23, Found 1092.45 ([M+2]⁺⁺/2).

### Example 7-20

### Preparation 6^{A},6^{D}-dideoxy-6^{A},6^{D}-di(Gly-Arg-Arg-Arg-Gly-Lys-Lys-Lys-NH₂)amino-β-cyclodextrin (25q)

Compound 25q was synthesized as described in the general procedures for the formation of CD-peptide bond and the subsequent deprotection of Boc group using compound 25p (0.043 g, 0.02 mmol) and Boc-Lys(Boc)-Lys(Boc)-Lys(Boc)-Gly-OH (0.037 g, 0.044 mmol, 2.2 eq) to yield product 25q (20 mg, 21%) as a pale yellow solid. ¹H-NMR (300 MHz, D₂O) δ 1.15-2.00 (m, 60H), 3.00-4.25 (m, 86H), 4.95(s, 7H); MS m/z Calcd for C₁₂₂H₂₂₈N₄₂O₄₉ 3067.37, Found 1023.28 ([M+3]⁺⁺⁺/3).

### Example 7-21

### Preparation 6^{A},6^{D}-dideoxy-6^{A},6^{D}-di[Gly-Lys(Boc)-Lys(Boc)-Lys(Boc)-Boc]amino-nonadecakis-O-benzyl-β-cyclodextrin (25r)

To a solution of 6^{A},6^{D}-dideoxy-6^{A},6^{D}-diamino-nonadecakis-O-benzyl-β-cyclodextrin (8) (0.1 g, 0.035 mmol) in anhydrous DMF (5 mL) were added HOBt (10.8 mg, 0.08 mmol), compound Boc-Lys(Boc)-Lys(Boc)-Lys(Boc)-Gly-OH (0.072 g, 0.084 mmol, 2.4 eq) and DCC (0.017 g, 0.084 mmol, 2.4 eq). The resulting solution was stirred at ambient temperature for 24 hours. The solvent was evaporated to dryness and the residue was dissolved in water/ethyl acetate and filtered. The organic phase was washed with water and brine. The solution was dried (MgSO₄), filtered and evaporated. The residue was purified by column chromatography on silica gel column using dichloromethane as an eluent to provide product 25r (100 mg, 63%). ¹H-NMR (300 MHz, CDCl₃) δ 1.10-2.00 (m, 108H), 2.85-5.25 (m, 123H), 6.90-7.40(m, 95H).

### Example 7-22

### Preparation 6^{A},6^{D}-dideoxy-6^{A},6^{D}-di[Gly-Lys(Boc)-Lys(Boc)-Lys(Boc)-Boc]amino-β-cyclodextrin (25s)

To a solution of compound 25r (0.3 g, 0.066 mmol) in 11 mL of mixed solvent of ethanol and acetic acid (10:1) was added 10% Pd/C (350 mg). The suspension was purged with nitrogen and stirred under hydrogen (balloon) at room temperature for one day. The reaction mixture was filtered through a cellite pad and washed with methanol and water. The filtrate was evaporated and the residue was washed with cyclohexane. The product was dried under vacuum to provide product 25s (110 mg, 66%). ¹H-NMR (300 MHz, CD₃OD) δ 1.10-2.00 (m, 108H), 2.85-4.25 (m, 64H), 4.95 (s, 7H).

### Example 7-23

### Preparation 6^{A},6^{D}-dideoxy-6^{A},6^{D}-di(Gly-Lys-Lys-Lys-NH₂)amino-β-cyclodextrin (25t)

A solution of compound 25s (0.1 g, 0.036 mmol) in a mixed solvent of trifluoroaceticacid (TFA, 3 mL) and dichloromethane (1 mL) was stirred at ambient temperature for 3 hours. The solvent was evaporated to provide a quantitative yield of product 25t as a TFA salt. ¹H-NMR (300 MHz, D₂O) δ 1.10-2.00 (m, 36H), 2.85-4.25 (m, 64H), 4.95 (s, 7H). MS m/z Calcd for C₈₂H₁₅₀N₁₆O₄₁ 2016.15, Found 1008.67 ([M+2]⁺⁺/2).

### Example 8

### Synthesis of Oligoamine-Cyclodextrin Conjugates 25u to 25z

Similar to the synthesis of oligopeptide-cyclodextrin conjugates, oligoamines were used as the cationic arms to prepare oligoamine-cyclodextrin conjugates. Reaction of compound 3 with the unprotected amine of an oligoamine afforded compounds 25u to 25z. Upon removal of protecting groups such as Boc or Cbz, the desired constructs suitable to complex with siRNA can be readily prepared.

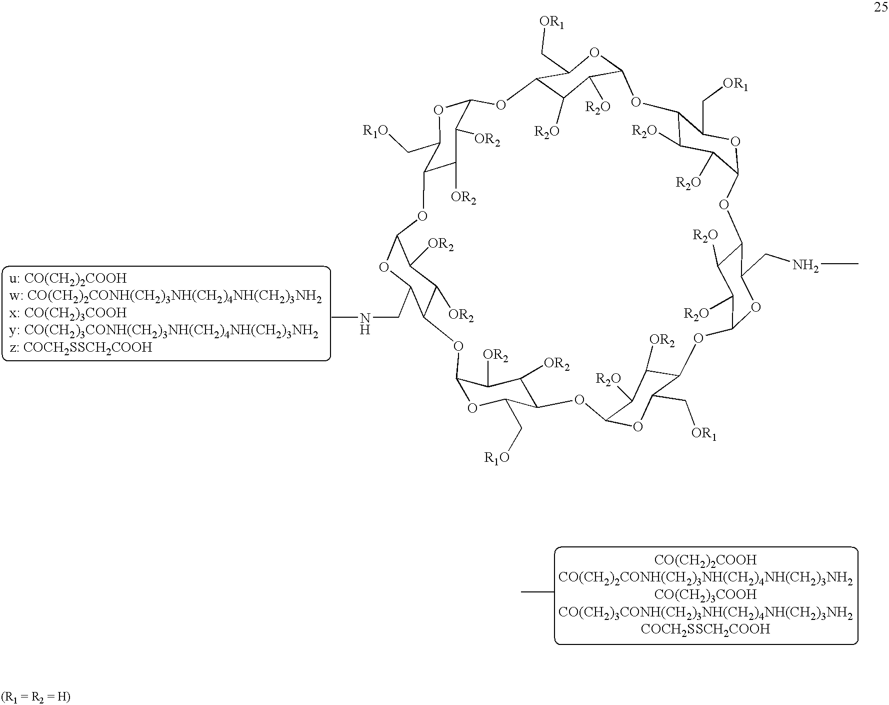

### Example 8-1

### Preparation of Compound 25u

To a solution of 3 (0.500 g, 0.440 mmols) in DMF (8 mL) was added succinic anhydride (0.093 g, 0.933 mmols) at room temperature and under nitrogen. Stirring was continued for 1 h, concentrated to ˜3 mL volume and acetone was added. The precipitate formed was further washed with acetone and dried under vacuum at 50° C. overnight to give 25u (0.570 g. 97%) as an off-white solid. ¹H NMR (300 MHz, D₂O): δ 2.30-2.65 (m, 11H), 3.05-3.40 (m, 5H), 3.40-3.65 (m, 18H), 3.65-3.95 (m, 47H), 4.95-5.10 (s, 7H).

### Example 8-2

### Spermine Coupling to Succinamide-Cyclodextrin—Preparation of Compound 25v

To a solution of 25u (0.160 g, 0.120 mmols) and H₂N(CH₂)₃NHBoc(CH₂)₄NHBoc(CH₂)₃NHBoc (0.145 g, 0.288 mmols) in DMF (6 mL) under nitrogen was added HOBt (0.039 g, 0.288 mmols) and DCC (0.059 g, 0.288 mmols) at room temperature and stirred for 4 h. Thereafter, HOBt (0.039 g, 0.288 mmols) and DCC (0.059 g, 0.288 mmols) were added and the reaction stirred at room temperature overnight, concentrated to near dryness under vacuum and the residue treated with dichloromethane. The precipitate obtained was further washed with dichloromethane several times and dried under vacuum at room temperature overnight to give 25v (0.138 g, 50%) as an off-white solid). ¹H NMR (300 MHz, DMSO-d₆): δ 1.30-1.50 (s, 54H), 1.50-1.8 (m, 12H), 2.15-2.45 (m. 9H), 2.80-3.25 (m, 25H), 3.50-3.80 (bs, 24H), 4.35-4.52 (bs, 5H), 4.52-5.00 (bs, 9H), 5.55-6.10 (bs, 15H), 6.60-6.80 (bs, 3H), 7.55-7.85(m, 4H).

### Example 8-3

### Preparation of Compound 25w

To the above compound 25v (0.124 g, 0.054 mmols) was added 75% TFA in dichloromethane (5 mL) and stirred at room temperature for 3h. The mixture was concentrated under vacuum, treated with water and extracted with dichloromethane (5 mL×2). The aqueous solution was lyophilized to give 0.070 g 76%) of 25w as an off-white solid. ¹H NMR (300 MHz, DMSO-d₆): δ 1.40-1.80 (bs, 11H), 1.90 (s, 6H), 2.10-2.45 (m, 9H), 2.65-3.20 (m, 26H), 3.50-4.00 (hs, 28H), 4.50-4.70 (hs, 6H), 4.85 (s, 9H), 5.40-6.15 (bs, 15H), 7.70 (s. 2H), 7.80-8.30 (m, 8H), 8.45-9.10 (m, 8H).

### Example 8-4

### Preparation of Compound 25x

To a solution of 3 (0.500 g, 0.440 mmols) in DMF (3 mL) was added glutaric anhydride (0.127 g, 1.113 mmols) at room temperature and under nitrogen. Stirring was continued for 2.5 h, concentrated to near dryness and added ethyl acetate. The precipitate formed was further washed with ethyl acetate and dried under vacuum at 60° C. for 2 h to give 25x (0.574 g, 96%) as an off-white solid, ¹H NMR (300 MHz, DMSO-d₆): δ 1.50-1.90 (m, 6H), 2.00-2.30 (m, 10H), 3.50-3.95 (bs, 30H), 4.20-4.70 (m, 6H), 4.85 (s, 9H), 5.30-6.20 (bs, 18H), 7.40-7.80 (m, 3H).

### Example 8-5

### Preparation of Compound 25y

Compound 25y was synthesized as described in the procedure for the coupling of spermine to derivatized cyclodextrin (see above for Step A and B) and the subsequent removal of the Boc group using compound 25x (0.200 g, 0.147 mmols), H₂N(CH₂)₃NHBoc(CH₂)₄NHBoc(CH₂)₃NHBoc (0.177 g, 0.353 mmols), HOBt (0.059 g, 0.441 mmols) and DCC (0.091 g, 0.441 mmols) to give 25y (0.124 g, 88%) as an off-white solid. ¹H NMR (300 MHz, DMSO-d₆): δ 1.30-1.80 (m, 15H), 1.90 (s, 3H), 2.10 (s, 7H), 2.60-3.20 (bs, 22H), 3.40 (s, 15H), 3.80-4.60(b, 26H), 4.85 (s, 9H), 5.30-6.20 (b, 14H), 7.45-7.80 (m, 3H), 7.97(s, 9H), 8.40-9.10 (m, 9H).

### Example 8-6

### Preparation of Compound 25z

To a solution of 3 (0.400 g, 0.353 mmols) and dithiodiglycolic acid (0.322 g, 1.760 mmols) in DMF (10 mL) under nitrogen was added HOBt (0.114 g, 0.847 mmols) and DCC (0.175 g, 0.847 mmols) at room temperature and stirred for 5 h, concentrated to near dryness under vacuum and the residue treated with absolute ethanol. The precipitate obtained was sonicated, filtered and further washed with absolute ethanol several times and dried under vacuum at 55° C. overnight. The crude product was purified on reverse HPLC (Phenomenex Luna 5u, C18(2) column) to give 25z (0.064 g, 12%) as an off-white solid). MS m/z Calcd for C₅₀H₈₀N₂O₃₉S₄ 1461.42, Found 1461.98.

### Example 9

### Synthesis of Oligopeptide-Cyclodextrin Conjugates 26-71

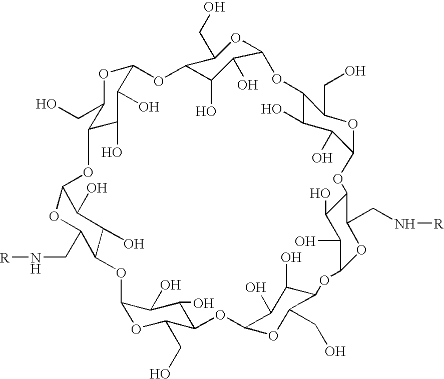

26: R = GGK
27: R = GGKK
28: R = GGKKK
29: R = GGKGK
30: R = GGOOOO
31: R = GGK_{(d)}KK_{(d)}K
32: R = GGKKKKK
33: R = GGKKKGK
34: R = GGOOOOOO
35: R = GGKKKKKK
36: R = GGKKKKKKG
37: R = GGKKKGKKKK
38: R = GGKKKKKKA_{(R)}
39: R = GGKKKKKKH
40: R = GGKKKKKKKK
41: R = GGK_{(d)}KK_{(d)}KK_{(d)}KK_{(d)}K
42: R = GGKKKKKKK_{(d)}G
43: R = GGKGKGKGKGK
44: R = GGKKKKKKKKK
45: R = GGKKKKKKKGRG
46: R = GGKKKKKKGKKKK
47: R = GGK_{(d)}K_{(d)}K_{(d)}GKGKGK
48: R = GGKKKKKK-CO(CH₂)₃NH₂
49: R = GGKKKKKK-CO(CH₂)₅NH₂
50: R = GGK(-COCH₂OC₂H₄OMe)KKKK
51: R = GGK(-COCH₂OC₂H₄OMe)KKKKGKKKK
52: R = GPKKK
53: R = GGKKKKKK-COCH₂NMe₂
54: R = GGKKK-CO(CH₂)₁₄CH₃
55: R = GGKKKGKKKK-CO(CH₂)₁₄CH₃
56: R = GGKKKGKKKK-dPEG₈
57: R = GGKKKGKKKK-CO(CH₂)₆CH₃
58: R = GGKKKKKKKK-CO(CH₂)₁₄CH₃
59: R = GGKKKKKK-CO(CH₂)₁₄CH₃
60: R = GGKKKGKKKK-PEG₄₀
61: R = GGKKKKKKKK-PEG₄₀
62: R = GGKKKGKKKK-CO(CH₂)₄CH₃
63: R = GGKKKKKKKK-CO(CH₂)₄CH₃
64: R = GGKKKKKK-COCH₂(OC₂H₄)₂Ome
65: R = GGKKKGKKKK-CO(CH₂)₆CH═CH(CH₂)₆CH₃
66: R = GGKKKKKKKK-CO(CH₂)₆CH═CH(CH₂)₆CH₃
67: R = GGKKKKKKKK-CO(CH₂CH₂O)₂₄(CH₂)₂NHCO(CH₂)₂-MAL
68: R = GGKKK-L1-CYGRKKRRQRRR
69: R = GGKKKGKKKK-L1-CYGRKKRRQRRR
70: R = GGKKKKKKKK-L1-CKKKGKKKGKKKGKKKGKKK
71: R = GGKKKGKKKK-dPEG₂₄-L1-CYGRKKRRQRRR
A = Alanine;
C = Cysteine;
G = Glycine;
H = Histdine;
K = Lysine;
O = Ornithine;
P = Proline;
Q = Glutamine;
R = Arginine;
Y = Tyrosine;
PEG = Polyethylene glycol;
MAL = Malenimide;
L1 =

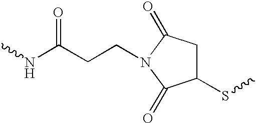

### Example 9-1

### General Procedure A: Formation of Peptide Bond

To a solution of 25f or 25h (1 eq) and C-terminus oligopeptide building block or simple amino acid with all amino group protected by t-butyl carbamate (Boc) or 9-fluorenylmethyl carbamate (Fmoc) (2.2 eq) in anhydrous DMF at room temperature was added coupling agents (DIC or TBTU or HATU and HObt) (2.2 eq) and diisopropylamine (DIPEA) (2.2 eq). The resulting solution was stirred at ambient temperature until completion (monitored by HPLC). The solution was concentrated under reduced pressure. The residue was washed with water and ethyl acetate. The compound was further purified by preparative HPLC if necessary. Refer to the general procedure in Example 7-1 if DCC was used as the coupling agent.

### Example 9-2

### General Procedure B: Deprotection of Fmoc Protected Amino Group

The Fmoc protected amino compound was dissolved in 20% piperidine/DMF. The resulting solution was stirred at room temperature for 0.5-1 hour until the protecting group was completely removed (monitored by HPLC). The solvent was removed under reduced pressure and the residue was mixed with water to form a slurry. The resulting slurry was filtered, and the filtrate was washed with ethyl acetate and dried to give the desired product. The product was used to the next step without further purification.

### Example 9-3

### General Procedure C: Deprotection of Boc Protected Amino Group

The Boc protected amino compound was dissolved in methylene chloride-trifluoroacetic acid solution (1:3). The resulting solution was stirred at rt for 0.5-1 hour. The solvent was then evaporated under reduced pressure to give a TFA salt. If necessary, the TFA salt can be converted to a HCl salt by dissolving the compound in 1 M HCl solution and then evaporated to dryness two times. The overall yields from coupling to the final product were from 5% to 90%. The products were further purified by preparative HPLC, if needed.

### Example 9-4

### General Procedure D for Coupling with Alkyl Carboxylic Acid (or Activated NHS Ester)

The same procedure in Example 9-1 was used to couple with alkylcarboxylic acids or NHS activated esters in the presence of DIPEA (2.2 eq) in DMF.

### Example 9-5

### General Procedure E: Coupling with Cross Linking Reagent

The oligopeptide-cyclodextrin with free amino groups at the end of each peptide (1 eq) was dissolved in DMF, after the cross linking reagent (NHS-R-MAL) (2.5 eq) and DIPEA (2.5 eq) were added to the reaction solution, the resulting reaction mixture was stirred at room temperature until completion of the reaction (monitored by HPLC). The reaction solution was concentrated under reduced pressure and the residue was washed with water and ethyl acetate. The crude product was used without further purification.

### Example 9-6

### General procedure F: Reaction Between Maleinmide Group and Thiol Group

The oligopeptide-cyclodextrin with maleinmide group (1 eq) was dissolved in a mixed solvent of methanol-1 M Tris buffer (pH 7.2) (ratio 4:1). The solution was degassed and the peptide with a free thiol group (2.5 eq) was added to the solution. After the reaction was complete (monitored by HPLC), the solvent was removed and the residue was purified by preparative HPLC to give product.

### Example 9-7

### Preparation of Compound 26

Compound 26 was synthesized using the general procedures described above as follows: coupled 25h with Fmoc-Lys(Boc)OH (procedure A); Fmoc deprotection (procedure B); Boc deprotection (procedure C). The compound 26 was isolated as the HCl salt, ¹HNMR (300 MHz, D₂O): δ 1.25-1.90 (m, 12H), 2.80-2.95 (m, 4H), 3.25-4.25 (m, 52H), 4.95(br, 7H); MS (MALDI) m/z calcd. for C₆₂H₁₀₈N₁₀O₃₉ 1616, Found 1615.

### Example 9-10

### Preparation of Compound 27

Compound 27 was synthesized using the general procedures described above as follows: coupled 25h with Fmoc-Lys(Boc)-Lys(Boc)-OH (procedure A); Fmoc deprotection (procedure B); Boc deprotection (procedure C). The compound 27 was isolated as an off-white solid of the TFA salt. ¹HNMR (300 MHz, D₂O): δ 1.25-1.90 (m, 24H), 2.80-2.95 (m, 8H), 3.25-4.25 (m, 54H), 4.95(br, 7H); MS (MALDI) m/z calcd for C₇₄H₁₃₂N₁₄O₄₁ 1872, Found 1895.

### Example 9-11

### Preparation of Compound 28

Compound 28 was synthesized using the general procedures described above as follows: coupled 25h with Fmoc-Lys(Boc)-Lys(Boc)-Lys(Boc)-OH (procedure A); Fmoc deprotection (procedure B); and Boc deprotection (procedure C). The compound 28 was isolated as an off-white solid of thc TFA salt. ¹HNMR (300 MHz, D₂O): δ 1.25-1.90 (m, 36H), 2.80-2.95 (m, 10H), 3.25-4.25 (m, 56H), 4.95(br, 7H); MS (MALDI) m/z calcd for C₈₆H₁₅₆N₁₈O₄₃ 2129, Found 2129.

### Example 9-12

### Preparation of Compound 29

Compound 29 was synthesized as described above using the general procedures of as follows: coupled 25f with Fmoc-Lys(Boc)-Gly-Lys(Boc)-Gly-OH (procedure A); Fmoc deprotection (procedure B); and Boc deprotection (procedure C). The compound 29 was isolated as an off-white solid of a TFA salt. MS (MALDI) m/z calcd for C₇₈H₁₃₈N₁₆O₄₃ 1987, Found 1989.

### Example 9-13

### Preparation of Compound 30

Compound 30 was synthesized using the general procedures described above as follows: coupled 25h with Fmoc-Om(Boc)-Om(Boc)-OH (procedure A); Fmoc deprotection (procedure B); Further coupled with Fmoc-Om(Boc)-Om(Boc)-OH (procedure A); Fmoc deprotection (procedure B); Boc deprotection (procedure C). The compound 30 was isolated as an off-white solid of the TFA salt. ¹HNMR (300 MHz, D₂O): δ 1.5-1.90 (m, 32H), 2.85-3.00 (m, 16H), 3.25-4.25 (m, 58H), 4.95(br, 7H); MS (MALDI) m/z calcd for C₉₀H₁₆₄N₂₂O₄₅ 2274, Found 2297.

### Example 9-14

### Preparation of Compound 31

Compound 31 was synthesized using the general procedures described above as follows: coupled 25h with Fmoc-Lys(Boc)-d-Lys(Boc)-Lys(Boc)-d-Lys(Boc)-OH (procedure A); Fmoc deprotection (procedure B); Boc deprotection (procedure C). The compound 31 was isolated as an off-white solid of the TFA salt. ¹HNMR (300 MHz, D₂O): δ 1.25-1.95 (m, 48H), 2.75-2.95 (m, 16H), 3.25-4.25 (m, 58H), 4.85-4.95(br, 7H); MS (MALDI) m/z calcd for C₉₈H₁₈₀N₂₂O₄₅ 2386, Found 2387.

### Example 9-15

### Preparation of Compound 32

Compound 32 was synthesized using the general procedures described above as follows: coupled 25h with Fmoc-Lys(Boc)-Lys(Boc)-Lys(Boc)-OH (procedure A); Fmoc deprotection (procedure B); further coupled with Boc-Lys(Boc)-Lys(Boc)-OH (procedure A); Boc deprotection (procedure C). The compound 32 was isolated as an off-white solid of the TFA salt. ¹HNMR (300 MHz, D₂O): δ 1.25-1.95 (m, 60H), 2.75-2.95 (m, 20H), 3.25-4.25 (m, 60H), 4.85-4.95 (br, 7H); MS (MALDI) m/z calcd for C₁₁₀H₂₀₄N₂₆O₄₇ 2642, Found 2667.

### Example 9-16

### Preparation of Compound 33

Compound 33 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Gly-OH (procedure A); Fmoc deprotection (procedure B), further coupled with Fmoc-Lys(Boc)-OH (procedure A), Fmoc deprotection (procedure B), Boc deprotection (procedure C). The compound 33 was isolated as an off-white solid of the HCl salt. ¹HNMR (300 MHz, D₂O): δ 1.25-1.95 (m, 48H), 2.75-2.95 (m, 16H), 3.25-4.25 (m, 60H), 4.85-5.00 (br, 7H); MS (MALDI) m/z calcd for C₁₀₂H₁₈₀N₂₂O₄₅ 2500, Found 2522.

### Example 9-17

### Preparation of Compound 34

Compound 34 was synthesized using the general procedures described above steps as follows: coupled 25h with Fmoc-Om(Boc)-Om(Boc)-OH (procedure A); Fmoc deprotection (procedure B); further coupled with Fmoc-Om(Boc)-Om(Boc)-OH (procedure A); Fmoc deprotection (procedure B); further coupled with Fmoc-Om(Boc)-Om(Boc)-OH (procedure A); Fmoc deprotection (procedure B); Boc deprotection (procedure C). The compound 34 was isolated as a solid of the TFA salt. ¹HNMR (300 MHz, D₂O): δ 1.25-1.95 (m, 48H), 2.75-2.95 (m, 24H), 3.25-4.25 (m, 62H), 4.85-5.00(br, 7H); MS (MALDI) m/z calcd for C₁₁₀H₂₀₄N₃₀O₄₉ 2730, Found 2756.

### Example 9-18

### Preparation of Compound 35

Compound 35 was synthesized using the general procedures described above as follows: coupled 25h with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-OH (procedure A); Hoc deprotection (procedure C). The compound 35 was isolated as a solid of the TFA salt. ¹HNMR (300 MHz, D₂O): δ 1.25-1.95 (m, 72H), 2.75-2.95 (m, 24H), 3.25-4.25 (m, 62H), 4.85-5.00(br, 7H); MS (MALDI) m/z calcd for C₁₂₂H₂₂₈N₃₀O₄₉ 2898, Found 2921.

### Example 9-19

### Preparation of Compound 36

Compound 36 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Gly-OH (procedure A); Boc deprotection (procedure C). The compound 36 was isolated as an off-white solid of the TFA salt: ¹HNMR (300 MHz, D₂O): δ 1.25-1.95 (m, 72H), 2.75-2.95 (m, 24H), 3.25-4.25 (m, 66H), 4.85-5.00(br, 7H); MS (MALDI) m/z calcd for C₁₂₆H₂₃₄N₃₂O₅₁ 3012. Found 3016.

### Example 9-20

### Preparation of Compound 37

Compound 37 was synthesized using the general procedures described above as follows: coupled 25h with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 37 was isolated as a solid of the TFA salt. ¹HNMR (300 MHz, D₂O): δ 1.25-1.95 (m, 84H), 2.75-2.95 (m, 28H), 3.25-4.25 (m, 68H), 4.85-5.00(br, 7H); MS (MALDI) m/z calcd for C₁₃₈H₂₅₈N₃₆O₅₃ 3269, Found 3294.

### Example 9-21

### Preparation of Compound 38

Compound 38 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-β-Ala-OH (procedure A); Boc deprotection (procedure C). The compound 38 was isolated as the TFA salt. ¹HNMR (300 MHz, D₂O): δ 1.25-1.95 (m, 72H), 2.65 (t, 4H), 2.75-2.95 (m, 24H), 3.15 (t, 4H), 3.25-4.25 (m, 62H), 4.85-5.00(br, 7H); MS (MALDI) calcd for C₁₂₈H₂₃₈N₃₂O₅₁ 3041, Found 3068.

### Example 9-22

### Preparation of Compound 39

Compound 39 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-His(Boc)-OH (procedure A); Boc deprotection (procedure C). The compound 39 was isolated as the TFA salt. ¹HNMR (300 MHz, D₂O): δ 1.25-1.95 (m, 72H), 2.75-2.95 (m, 25H), 3.25-4.25 (m, 64H), 4.85-5.00 (br, 7H), 7.3 (s, 2H), 8.55 (s, 2H); MS (MALDI) m/z calcd for C₁₃₄H₂₄₂N₃₆O₅₁ 3173, Found 3195.

### Example 9-23

### Preparation of Compound 40

Compound 40 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Lys(Boc)-Lys(Boc)-OH (procedure A); Boc deprotection (procedure C). The compound 40 was isolated as the TFA salt. ¹HNMR (300 MHz, D₂O): δ 1.25-1.95 (m, 96H), 2.75-2.95 (m, 32H), 3.25-4.25 (m, 66H), 4.85-5.00 (br, 7H).

### Example 9-24

### Preparation of Compound 41

Compound 41 was synthesized using the general procedures described above as follows: coupled 25h with Fmoc-Lys(Boc)-d-Lys(Boc)-Lys(Boc)-d-Lys(Boc)-OH (procedure A); Fmoc deprotection (procedure B); further coupled with Fmoc-Lys(Boc)-d-Lys(Boc)-Lys(Boc)-d-Lys(Boc)-OH (procedure A); Fmoc deprotection (procedure B); Boc deprotection (procedure C). The compound 41 was isolated as the TFA salt. ¹HNMR (300 MHz, D₂O): δ 1.25-1.95 (m, 96H), 2.75-2.95 (m, 32H), 3.25-4.25 (m, 66H), 4.85-5.00 (br, 7H); MS (MALDI) m/z calcd for C146H₂₇₆N₃₈O₅₃ 3412. Found 3435.

### Example 9-25.

### Preparation of Compound 42

Compound 42 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Fmoc-Gly-d-Lys(Boc)-OH (procedure A); Fmoc deprotection (procedure B); Boc deprotection (procedure C). The compound 42 was isolated as the TFA salt. ¹HNMR (300 MHz, D₂O): δ 1.25-1.95 (m, 84H), 2.75-2.95 (m, 28H), 3.25-4.25 (m, 68H), 4.85-5.00 (br, 7H); MS (MALDI) m/z calcd for C₁₃₈H₂₅₈N₃₆O₅₃ 3272, Found 3270.

### Example 9-26

### Preparation of Compound 43

Compound 43 was synthesized using the general procedures described above as follows: coupled 25f with Fmoc-Lys(Boc)-Gly-Lys(Boc)-Gly-OH (procedure A); Fmoc deprotection (procedure B); further coupled with Boc-Lys(Boc)-Gly-Lys(Boc)-Gly-Lys(Boc)-Gly-OH (procedure A); Boc deprotection (procedure C). The compound 43 was isolated as the TFA salt. ¹HNMR (300 MHz, D₂O): δ 1.25-1.95 (m, 60H), 2.75-2.95 (m, 20H), 3.25-4.25 (m, 76H), 4.85-5.00 (br, 7H); MS (MALDI) m/z calcd for C₁₂₆H₂₂₈N₃₄O₅₅ 3098, Found 3122.

### Example 9-27

### Preparation of Compound 44

Compound 44 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-OH (procedure A); Boc deprotection (procedure C). The compound 44 was isolated as the TFA salt. ¹HNMR (300 MHz, D₂O): δ 1.25-1.95 (m, 108H), 2.75-2.95 (m, 36H), 3.25-4.25 (m, 68H), 4.85-5.00 (br, 7H). MS (MALDI) m/z calcd for C₁₃₈H₂₅₈N₃₄O₅₅ 3668, Found 3689.

### Example 9-28

### Preparation of Compound 45

Compound 45 was synthesized using the general procedures described above as follows: coupled between 25h and Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Fmoc-Gly-Arg-Gly-Lys(Boc)-OH (procedure A); Fmoc deprotection (procedure B); Boc deprotection (procedure C). The compound 45 was isolated as the TFA salt. ¹HNMR (300 MHz, D₂O): δ 1.25-1.95 (m, 92H), 2.75-2.95 (m, 28H), 3.1(t, 4H); 3.25-4.25 (m, 74H), 4.85-5.00 (br, 7H).

### Example 9-29

### Preparation of Compound 46

Compound 46 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 46 was isolated as the TFA salt. ¹HNMR (300 MHz, D₂O): δ 1.25-1.95 (m, 120H), 2.80-2.97 (m, 40H), 3.25-4.25 (m, 74H), 4.85-5.00 (br, 7H). MS (MALDI) m/z calcd for C₁₇₄H₃₃₀N48O₅₉ 4037, Found 4066.

### Example 9-30

### Preparation of Compound 47

Compound 47 was synthesized using the general procedures described above as follows: coupled 25h with Fmoc-d-Lys(Boc)-d-Lys(Boc)-d-Lys(Boc)-OH (procedure A); Fmoc deprotection (procedure B); further coupled with Fmoc-Lys(Boc)-Gly-Lys(Boc)-Gly-Lys(Boc)-Gly-OH (procedure A); Fmoc deprotection (procedure B); Boc deprotection (procedure C). The compound 47 was isolated as the TFA salt. ¹HNMR (300 MHz, D₂O): δ 1.25-1.95 (m, 72H), 2.80-2.95 (m, 24H), 3.25-4.25 (m, 72H), 4.85-5.00 (br, 7H).

### Example 9-31

### Preparation of Compound 48

Compound 48 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-NH(CH₂)₃COOH (procedure A); Boc deprotection (procedure C). The compound 48 was isolated as the TFA salt. ¹HNMR (300 MHz, D₂O): δ 1.25-1.90 (m, 76H), 2.30 (t, 4H), 2.80-2.95 (m, 28H), 3.30-4.25 (m, 62H), 4.90-5.00 (br, 7H). MS (MALDI) m/z calcd for C₁₃₀H₂₄₂N₃₂O₅₁ 3068, Found 3069.

### Example 9-32

### Preparation of Compound 49

Compound 49 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); further coupled with Fmoc-NH(CH₂)₅COOH (procedure A); Fmoc deprotection (procedure B); Boc deprotection (procedure C). The compound 49 was isolated as the HCl salt. ¹HNMR (300 MHz, D₂O): δ 1.25-1.90 (m, 84H), 2.20 (t, 4H), 2.80-2.95 (m, 28H), 3.30-4.25 (m, 62H), 4.90-5.00 (br, 7H).

### Example 9-33

### Preparation of Compound 50

Compound 50 was synthesized using the general procedures described above as follows: coupled 25h with Fmoc-Lys(COCH₂OCH₂CH₂OCH₃)—OH (procedure A); Fmoc deprotection (procedure B); further coupled with Fmoc-Lys(Boc)-Lys(Boc)-Lys(Boc)-Lys(Boc)-OH (procedure A); Fmoc deprotection (procedure B); Boc deprotection (procedure C). The compound 50 was isolated as the HCl salt: ¹HNMR (300 MHz, D₂O ): δ 1.25-1.90 (m, 60H), 2.80-2.95 (m, 16H), 3.15 (t, 4H), 3.30 (s, 6H), 3.35-4.25 (m, 62H), 4.90-5.00 (br, 7H).

### Example 9-34

### Preparation of Compound 51

Compound 51 was synthesized using the general procedures described above as follows: coupled 25h with Fmoc-Lys(COCH₂OCH₂CH₂OCH₃)—OH (procedure A); Fmoc deprotection (procedure B); further coupled with Fmoc-Lys(Boc)-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 51 was isolated as the HCl salt. ¹HNMR (300 MHz, D₂O): δ 1.15-1.85 (m, 108H), 2.80-2.95 (m, 32H), 3.15 (t, 4H), 3.30 (s, 6H), 3.35-4.25 (m, 80H), 4.90-5.00 (br, 7H); MS (MALDI) m/z calcd for C₁₇₂H₃₂₂N₄₄O₆₃ 4013, Found 4007

### Example 9-35

### Preparation of Compound 52

Compound 52 was synthesized as described in the above scheme using general procedures described above as follows: coupled 25f with Fmoc-Pro-OH (procedure A); Fmoc deprotection (procedure B); further coupled with Fmoc-Lys(Boc)-Lys(Boc)-Lys(Boc)-OH (procedure A); Fmoc deprotection (procedure B); Boc deprotection (procedure C). The compound 52 was isolated as the HCl salt. ¹HNMR (300 MHz, DMSO-d₆): δ 1.20-1.85 (m, 44H), 2.65-2.90 (m, 12H), 3.20-4.5 (m, 58H), 4.75-5.00 (br, 7H), 5.80(br, 14H), 7.75-8.25(b, 29H); MS (MALDI) m/z calcd for C₁₇₀H₁₆₄N₁₈O₄₃ 2210, Found 2233.

### Example 9-36

### Preparation of Compound 53

Compound 53 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); coupled with (Me)₂NCH₂COOH (procedure D); Boc deprotection (procedure C). The compound 53 was isolated as the TFA salt. ¹HNMR (300 MHz, D₂O): δ 1.25-1.95 (m, 72H), 2.75-2.95 (m, 36H), 3.20 (s, 4H), 3.25-4.25 (m, 62H), 4.85-5.00 (br, 7H); MS (MALDI) m/z calcd for C₁₃₈H₂₅₈N₃₆O₅₃ 3272, Found 3270.

### Example 9-37

### Preparation of Compound 54

Compound 54 was synthesized using the general procedures described above as follows: coupled 25h with Fmoc-Lys(Boc)-Lys(Boc)-Lys(Boc)-OH (procedure A); Fmoc deprotection (procedure B); coupled with CH₃(CH₂)₁₄COOH (procedure D); Boc deprotection (procedure C). The compound 54 was isolated as the HCl salt. ¹HNMR (300 MHz, D₂O): δ 0.76(t, 6H), 1.10-1.80 (m, 88H), 2.16 (t, 4H), 2.80-2.95 (m, 12H), 3.30-4.25 (m, 56H), 4.85-5.00 (br, 7H); MS (MALDI) m/z calcd for C₁₃₀H₂₄₂N₃₂₀₅₁ 3069, Found 3093.

### Example 9-38

### Preparation of Compound 55

Compound 55 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(CH₂)₁₄COOH (procedure D); Boc deprotection (procedure C). The compound 55 was isolated as the HCl salt. ¹HNMR (300 MHz, D₂O): δ 0.75 (t, 6H), 1.05-1.75 (m, 136H), 2.20 (t, 4H), 2.75-2.95 (m, 28H), 3.25-4.25 (m, 68H), 4.85-5.00 (br, 7H); MS (MALDI) m/z calcd for C₁₇₀H₃₁₈N₃₆O₅₅ 3745, Found 3769.

### Example 9-39

### Preparation of Compound 56

Compound 56 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(OCH₂CH₂)₈COOH (procedure D); Boc deprotection (procedure C). The compound 56 was isolated as the HCl salt. ¹HNMR (300 MHz, D₂O): δ 1.25-1.80 (m, 84H), 2.46 (t, 4H), 2.80-2.95 (m, 28H), 3.22 (s, 6H), 3.25-4.25 (m, 128H), 4.85-5.00 (br, 7H); MS (MALDI) m/z calcd for C₁₇₄H₃₂₆N₃₆O₇₁ 4057, Found 4081.

### Example 9-40

### Preparation of Compound 57

Compound 57 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(CH₂)₆COOH (procedure D); Boc deprotection (procedure C). The compound 57 was isolated as the HCl salt. ¹HNMR (300 MHz, D₂O): δ 0.75 (t, 6H), 1.05-1.80 (m, 104H), 2.20 (t, 4H), 2.75-2.95 (m, 28H), 3.25-4.25 (m, 68H), 4.85-5.00 (br, 7H); MS (MALDI) m/z calcd for C₁₅₄H₂₈₆N₃₆O₅₅ 3521, Found 3521.

### Example 9-41

### Preparation of Compound 58

Compound 58 was synthesized as described in the above scheme using the general procedures of steps a-d as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with CH₃(CH₂)₁₄COOH (procedure D); Boc deprotection (procedure C). The compound 58 was isolated as the HCl salt. ¹HNMR (300 MHz, D₂O): δ 0.75 (t, 6H), 1.05-1.80 (m, 148H), 2.20 (t, 4H), 2.75-2.95 (m, 32H), 3.25-4.25 (m, 66H), 4.85-5.00 (br, 7H); MS (MALDI) m/z calcd for C₁₇₈H₃₉₆N₃₈O₅₅ 3888.

### Example 9-42

### Preparation of Compound 59

Compound 59 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); coupled with CH₃(CH₂)₁₄COOH (procedure D); Boc deprotection (procedure C). The compound 59 was isolated as the TFA salt. ¹HNMR (300 MHz, D₂O): δ 0.80(t, 6H), 1.20-1.90 (m, 124H), 2.20 (t, 4H), 2.80-2.95 (m, 24H), 3.30-4.25 (m, 66H), 4.90-5.00 (br, 7H); MS (MALDI) m/z calcd for C₁₅₄H₂₈₈N₃₀O₅₁ 3376, Found 3400.

### Example 9-43

### Preparation of Compound 60

Compound 60 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with m-PEG₄₀-NHS (Mₚ=1892 Dalton) (procedure D); Boc deprotection (procedure C). The compound 60 was isolated as a mixture of mono and disubstituted product as the HCl salts: ¹HNMR (300 MHz, D₂O): δ 1.20-1.90 (m, 84H), 2.45 (s, 2.7H), 2.80-2.95 (m, 28H), 3.25 (s, 3.8H), 3.25-4.25 (m, 185H), 4.85-5.00 (br, 7H).

### Example 9-44

### Preparation of compound 61

Compound 61 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with m-PEG₄₀-NHS (Mₚ=1892 Dalton) (procedure D); Boc deprotection (procedure C). The compound 61 was isolated as a mixture of mono and disubstituted product as the HCl salts. ¹HNMR (300 MHz, D₂O): δ 1.20-1.90 (m, 96H), 2.45 (s, 6H), 2.80-2.95 (m, 32H), 3.25 (s, 9H), 3.27-4.25 (m, 387H), 4.85-5.00 (br, 7H).

### Example 9-45

### Preparation of Compound 62

Compound 62 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(CH₂)₄COOH (procedure D); Boc deprotection (procedure C). The compound 62 was isolated as the HCl salt. ¹HNMR (300 MHz, D₂O): δ 0.75 (t, 6H), 1.05-1.80 (m, 96H), 2.20 (t, 4H), 2.75-2.95 (m, 28H), 3.25-4.25 (m, 68H), 4.85-5.00 (br, 7H); MS (MALDI) m/z calcd for C₁₅₀H₂₇₈N₃₆O₅₅ 3466, Found 3460.

### Example 9-46

### Preparation of Compound 63

Compound 63 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with CH₃(CH₂)₄COOH (procedure D); Boc deprotection (procedure C). The compound 63 was isolated as the HCl salt. ¹HNMR (300 MHz, D₂O): δ 0.75 (t, 6H), 1.05-1.80 (m, 108H), 2.20 (t, 4H), 2.75-2.95 (m, 32H), 3.25-4.25 (m, 66H), 4.85-5.00 (br, 7H); MS (MALDI) m/z calcd for C₁₅₈H₂₉₆N₃₈O₅₅ 3608, Found 3611.

### Example 9-47

### Preparation of Compound 64

Compound 64 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-OH (procedure A); Fmoc deprotection (procedure B); coupled with CH₃(OCH₂CH₂)₂CH₂COOH (procedure D); Boc deprotection (procedure C). The compound 64 was isolated as the TFA salt. ¹HNMR (300 MHz, D₂O): δ 1.25-1.80 (m, 72H), 2.80-2.95 (m, 24H), 3.25 (s, 6H), 3.25-4.25 (m, 72H), 4.85-5.00 (br, 7H); MS (MALDI) m/z calcd for C₁₃₆H₂₅₂N₃₀O₅₇ 3219, Found 3244.

### Example 9-48

### Preparation of Compound 65

Compound 65 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(CH₂)₆CH═CH(CH₂)₆COOH (procedure D); Boc deprotection (procedure C). The compound 65 was isolated as the HCl salt, ¹HNMR (300 MHz, D₂O): δ 0.75 (t, 6H), 1.05-1.80 (m, 124H), 1.90 (t, 8H), 2.20 (t, 4H), 2.75-2.95 (m, 28H), 3.25-4.25 (m, 68H), 4.85-5.00 (br, 7H); MS (MALDI) m/z calcd for C₁₇₄H₃₂₂N₃₆O₅₅ 3798, Found 3819.

### Example 9-49

### Preparation of Compound 66

Compound 66 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with CH₃(CH₂)₆CH═CH(CH₂)₆COOH (procedure D); Boc deprotection (procedure C). The compound 66 was isolated as an HCl salt. ¹HNMR (300 MHz, D₂O): δ 0.75 (t, 6H), 1.05-1.80 (m, 124H), 1.90 (t, 8H), 2.20 (t, 4H), 2.75-2.95 (m, 28H), 3.25-4.25 (m, 68H), 4.85-5.00 (br, 7H); MS (MALDI) m/z calcd for C₁₈₂H₃₄₀N₃₈O₅₅ 3940, Found 3939.

### Example 9-50

### Preparation of Compound 67

Compound 67 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with MAL-dPEG₂₄-NHS (procedure E); Boc deprotection (procedure C). The compound 67 was isolated as the HCl salt: ¹HNMR (300 MHz, D₂O): δ 1.20-1.80 (m, 96H), 2.3-2.50(t, 8H), 2.75-2.95 (m, 32H), 3.2-3.40(m, 8H), 3.25-4.25 (m, 258H), 4.85-5.00 (br, 7H); MS (MALDI) m/z calcd for C₂₆₂H₄₈₈N₄₂O₁₀₉ 5970, Found 5971.

### Example 9-51

### Preparation of Compound 68

Compound 68 was synthesized using the general procedures described above as follows: coupled 25h with Fmoc-Lys(Boc)-Lys(Boc)-Lys(Boc)-OH (procedure A); Fmoc deprotection (procedure B); further coupled with NHS-3-maleimideopropionate (procedure E); further coupled with CYGRKKRRQRRR (CTAT) (procedure F); Boc deprotection (procedure C). The compound 68 was isolated as the HCl salt. ¹HNMR (300 MHz, D₂O): δ 1.25-1.80 (m, 112H), 2.25(t, 8H), 2.3-2.50 (m, 4H), 2.80-2.95 (m, 20H), 3.0-3.20 (m, 24H), 3.25-4.25 (m, 96H), 4.85-5.00 (br, 7H); 6.70 (d, 4H), 7.05 (d, 4H); MS (MALDI) m/z calcd for C₂₃₄H₄₁₂N₈₆O₇₉S₂ 5758, Found 5755.

### Example 9-52

### Preparation of Compound 69

Compound 69 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with NHS-3-maleimideopropionate (procedure E); further coupled with CYGRKKRRQRRR (CTAT) (procedure F); Boc deprotection (procedure C). The compound 69 was isolated as the HCl salt. ¹HNMR (300 MHz, D₂O): δ 1.20-1.85 (m, 160H), 2.25(t, 4H), 2.3-2.50 (m, 8H), 2.80-2.95 (m, 36H), 3.0-3.20 (m, 24H), 3.25-4.25 (m, 108H), 4.85-5.00 (br, 7H); 6.70 (d, 4H), 7.05 (d, 4H); MS (MALDI) m/z calcd for C₂₈₆H₅₁₄N₁₀₄O₈₉S₂ 6898, Found 6889.

### Example 9-53

### Preparation of Compound 70

Compound 70 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with NHS-3-maleimideopropionate (procedure E); further coupled with CKKKGKKKGKKKGKKKGKKK (procedure F); Boc deprotection (procedure C). The compound 70 was isolated as the HCl salt. ¹HNMR (300 MHz, D₂O): δ 1.20-1.85 (m, 276H), 2.80-2.95 (m, 36H), 3.0-3.20 (m, 24H), 3.25-4.25 (m, 108H), 4.85-5.00 (br, 7H); 6.70 (d, 4H), 7.05 (d, 4H).

### Example 9-54

### Preparation of Compound 71

Compound 71 was synthesized using the general procedures described above as follows: coupled 25h with Fmoc-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with NHS-dPEG₂₄-MAL (procedure E); coupled with CYGRKKRRQRRR (CTAT) (procedure F); Boc deprotection (procedure C). The compound 71 was isolated as the HCl salt. ¹HNMR (300 MHz, D₂O): δ 1.20-1.85 (m, 160H), 2.25(t, 4H), 2.3-2.50 (m, 12H), 2.80-2.95 (m, 36H), 3.0-3.20 (m, 24H), 3.25-4.25 (m, 304H), 4.85-5.00 (br, 7H); 6.70 (d, 4H), 7.05 (d, 4H); MS (MALDI) m/z calcd for C₃₈₈H₇₁₆N₁₀₆O₁₃₉S₂ 9154, Found 9155.

### Example 10

### Synthesis of Oligopeptide-Cyclodextrin Conjugates 72-79

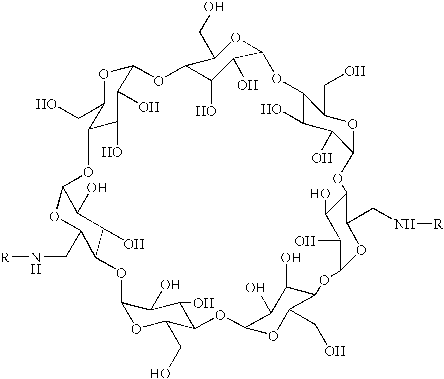

72: R = K(COOCH₂CH═CH₂)GKKKKGKKKK
73: R = KGKKKKGKKKK
74: R = K(PEG₄₀)GKKKKGKKKK
75: R = K(L1-m-dPEG₂₄)GKKKKGKKKK
76: R = K(m-dPEG₁₂)GKKKKGKKKK
77: R = K(dPEG₂₄-L1-CYGRKKRRQRRR)GKKKKGKKKK
78: R = K(dPEG₈-L1-CYGRKKRRQRRR)GKKKKGKKKK
79: R = K(L1-CYGRKKRRQRRR)GKKKKGKKKK
G = Glycine;
C = Cysteine;
K = Lysine;
Q = Glutamine;
R = Arginine;
Y = Tyrosine;
PEG = Polyethylene glycol;
L1 =

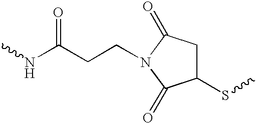

### Example 10-1

### General Procedure G: Deprotection of Alloc Protected Amino Group

Oligopeptide-cyclodextrin with an Alloc protected amino group (1 eq) was dissolved in DMF at room temperature. After the solution was degassed, Pd(Ph₃)₄ (2.05 eq) and Me₂NH/BH₃ (2.05 eq) were added to the solution. The mixture was stirred at room temperatures under positive nitrogen pressure overnight. After adding MeOH, the resulting mixture was filtered and the solid was washed with H₂O, NaHCO₃ and NH₄Cl solution and dried to provide the desired product with 50-90% yields.

### Example 10-2

### Preparation of Compound 72

Compound 72 was synthesized using the general procedures described in Examples 9 &10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 72 was isolated as the HCl salt. ¹HNMR (300 MHz, D₂O): δ 1.20-1.85 (m, 108H), 2.80-2.95 (m, 36H), 3.25-4.25 (m, 68H), 4.5 (m. 4H), 4.85-5.00 (n, 7H), 5.15 (d, 4H), 5.75-5.90 (m, 2H); MS (MALDI) m/z calcd for C₁₆₆H₃₀₈N₄₂O₅₉ 3836, Found 3833.

### Example 10-3

### Preparation of Compound 73

Compound 73 was synthesized using the general procedures described in Examples 9 &10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure G); Boc deprotection (procedure C). The compound 73 was isolated as the HCl salt. ¹HNMR (300 MHz, D₂O): δ 1.20-1.85 (m, 108H), 2.80-2.95 (m, 36H), 3.25-4.25 (m, 68H), 4.85-5.00 (br, 7H); MS (MALDI) m/z calcd for C₁₅₈H₃₀₀N₄₂O₅₅ 3668, Found 3670 (M+H)⁺.

### Example 10-4

### Preparation of Compound 74

Compound 74 was synthesized using the general procedures described in Examples 9 & 10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure G); coupled with NHS-m-PEG₄₀-NHS (Mₚ=1892 Dalton) (procedure E); Boc deprotection (procedure C). The compound 74 was isolated as the HCl salts of a mixture of mono- and di-PEG substituted products. ¹HNMR (300 MHz, D₂O): δ 1.20-1.85 (m, 108H), 2.80-2.95 (m, 32H), 3.00-3.10 (m, 4H), 3.25-4.25 (m, 265H), 4.85-5.00 (br, 7H); MS (MALDI) m/z has a distribution from 5320-6459 and 6720-8488.

### Example 10-5

### Preparation of Compound 75

Compound 75 was synthesized using the general procedures described in Examples 9 & 10 for each step: Coupled between 3 and Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure D); coupled with m-dPEG₂₄-NHS (procedure E), Boc deprotection (procedure C). The compound 75 was isolated as the HCl salts of a mixture of mono- and di-PEG substituted products. ¹HNMR (300 MHz, D₂O): δ 1.20-1.85 (m, 108H), 2.20 (t, 4H), 2.80-2.95 (m, 36H), 3.25-4.25 (m, 212H), 4.85-5.00 (br, 7H); MS (MALDI) m/z calcd for C₂₇₀H₅₁₂N₄₆O₁₀₉ 6147, Found 6168.

### Example 10-6

### Preparation of Compound 76

Compound 76 was synthesized using the general procedures described in Examples 9 & 10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure G); coupled with m-dPEG₁₂-NHS (procedure E); Boc deprotection (procedure C). The compound 76 was isolated as the HCl salt. ¹HNMR (300 MHz, D₂O): δ 1.20-1.85 (m, 108H), 2.20 (t, 4H), 2.80-2.95 (m, 36H), 3.25-4.25 (m, 134H), 4.85-5.00 (br, 7H); MS (MALDI) m/z calcd for C₁₉₄H₃₆₈N₄₂O₇₃ 4457, Found 4479.

### Example 10-7

### Preparation of Compound 77

Compound 77 was synthesized using the general procedures described in examples 9 & 10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure G); coupled with NHS-dPEG₂₄-MAL (procedure E); Boc deprotection (procedure C); coupled with CYGRKKRRQRRR (CTAT) (procedure F). The compound 77 was isolated as the HCl salt. ¹HNMR (300 MHz, D₂O): δ 1.20-1.85 (m, 184H), 2.20 (t, 4H), 2.80-2.95 (m, 44H), 3.05-3.25 (m, 24H), 3.25-4.25 (m, 450H), 4.85-5.00 (br, 7H), the aromatic peaks are buried in the noise; MS (MALDI) m/z calcd for C₄₀₈H₇₅₈N₁₁₂O₁₄₁S₂ 9553, Found 9564.

### Example 10-8

### Preparation of Compound 78

Compound 78 was synthesized using the general procedures described in examples 9 & 10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of alloc protecting group (procedure G); coupled with NHS-dPEG₈-MAL (procedure E); Boc deprotection (procedure C); coupled with CYGRKKRRQRRR (CTAT) (procedure F). The compound 78 was isolated as the HCl salts of a mixture of the desired compound and the dimer of CTAT. ¹HNMR (300 MHz, D₂O): δ 1.20-1,85 (m, 184H), 2.25 (t, 8H), 2.25-2.5 (m, 8H), 2.80-2.95 (m, 44H), 3.05-3.25 (m, 24H), 3.05-3.20 (m, 38H), 3.25-4.25 (m, 150H), 4.85-5.00 (br, 7H), 6.72 (d, 6H), 7.05 (d, 6H); MS (MALDI) m/z calcd for C₃₄₄H₆₃₀N₁₁₂O₁₀₉S₂ 8143, Found 8161.

### Example 10-9

### Preparation of Compound 79

Compound 79 was synthesized using the general procedures described in examples 9 & 10 for each step as follows: coupled 3 with Fmoc-Lys(Alloc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); further coupled with Boc-Lys(Boc)-Lys(Boc)-Lys(Boc)-Lys(Boc)-Gly-OH (procedure A); deprotection of allot protecting group (procedure G); coupled with NHS-propionate-MAL (procedure E); Boc deprotection (procedure C); coupled with CYGRKKRRQRRR (CTAT) (procedure F). The compound 79 was isolated as the HCl salts of a mixture of the desired compound and the dimer of CTAT.

### Example 11

### Synthesis of Oligopeptide-Cyclodextrin Conjugates 80-89

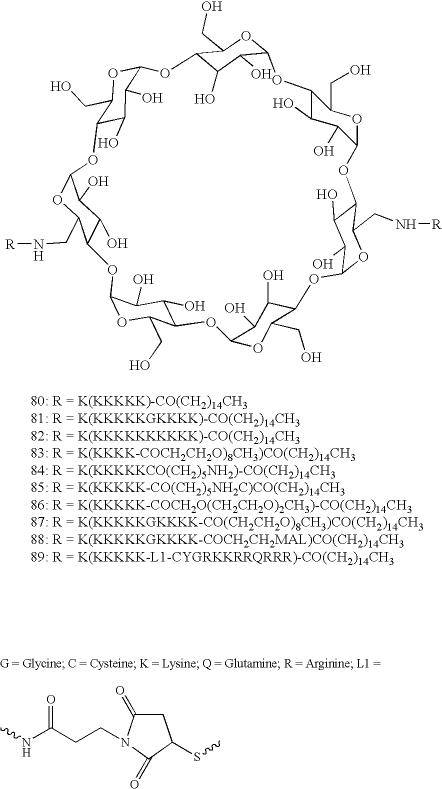

### Example 11-1

### Preparation of Compound 80

Compound 80 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 80 was isolated as the HCl salt. ¹HNMR (300 MHz, D₂O): δ 0.80 (t, 6H), 1.05-1.85 (m, 124H), 2.18 (b,4H), 2.80-2.95 (m, 20H), 3.00 (m 4H), 3.25-4.25 (m, 54H), 4.5 (m. 4H), 4.85-5.05 (br, 7H); MS (MALDI) m/z calcd for C₁₄₆H₂₇₆N₂₆O₄₇ 3147, Found 3167.

### Example 11-2

### Preparation of Compound 81

Compound 81 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); Boc deprotection (procedure C). The compound 81 was isolated as the HCl salt. ¹HNMR (300 MHz, D₂O): δ 0.80 (t, 6H), 1.00-1.90 (m, 172H), 2.18 (b,4H), 2.80-2.95 (m, 36H), 3.05 (t, 4H), 3.25-4.25 (m, 66H), 4.85-5.05 (br, 7H).

### Example 11-3

### Preparation of Compound 82

Compound 82 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); Boc deprotection (procedure C). The compound 82 was isolated as the HCl salt. ¹HNMR (300 MHz, D₂O): δ 0.80 (t, 6H), 1.00-1.90 (m, 184H), 2.18 (b,4H), 2.80-2.95 (m, 44H), 3.25-4.25 (m, 64H), 4.85-5.05 (br, 7H); MS (MALDI) m/z calcd for C₂₀₆H₃₉₆N₄₆O₅₇ 4429, Found 4426.

### Example 11-4

### Preparation of Compound 83

Compound 83 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with CH₃(CH₂CH₂O)₈NHS (procedure D); Boc deprotection (procedure C). The compound 83 was isolated as the HCl salt. ¹HNMR (300 MHz, D₂O): δ 0.80 (t, 6H), 1.00-1.90 (m, 172H), 2.18 (b,4H), 2.50 (t, 4H), 2.80-2.95 (m, 44H), 3.05 (t, 4H), 3.25-4.25 (m, 176H), 4.85-5.05 (br, 7H); MS (MALDI) m/z calcd for C₁₈₂H₃₄₄N₂₆O₆₅ 3936, Found 3935.

### Example 11-5

### Preparation of Compound 84

Compound 84 was synthesized as described in the above scheme using the general procedures in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with Fmoc-NH(CH₂)₅COOH (procedure A); Fmoc deprotection (procedure B); Boc deprotection (procedure C). The compound 84 was isolated as the HCl salt. ¹HNMR (300 MHz, D₂O): δ 0.80 (t, 6H), 1.00-1.90 (m, 136H), 2.18 (b,4H), 2.30(t, 4H), 2.80-2.95 (m, 24H), 3.05 (t, 4H), 3.25-4.25 (m, 54H), 4.85-5.05 (br, 7H); MS (MALDI) m/z calcd for C₁₅₈H₂₉₈N₂₈O₄₉ 3374, Found 3373.

### Example 11-6

### Preparation of Compound 85

Compound 85 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with Fmoc-NH(CH₂)₅COOH (procedure A); Fmoc deprotection (procedure B); coupled with Fmoc-Cys(trit)-OH (procedure A); Fmoc deprotection (procedure B); Boc deprotection (procedure C). The compound 85 was isolated as the HCl salt. ¹HNMR (300 MHz, D₂O): δ 0.80 (t, 6H), 0.8-1.90 (m, 136H), 2.18 (b, 4H), 2.30 (t, 4H), 2.80-2.95 (m, 24H), 3.05 (t, 4H), 3.25-4.25 (m, 61H), 4.85-5.05 (br, 7H); MS (MALDI) m/z calcd for C₁₆₄H₃₀₈N₃₀O₅₁S₂ 3580, Found 3581 (M+H)⁺.

### Example 11-7

### Preparation of Compound 86

Compound 86 was synthesized using the general procedures described in example 9 for each step as follows: coupled between 3 and Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with CH₃O(CH₂CH₂O)₂CH₂COOH (procedure A); Boc deprotection (procedure C). The compound 86 was isolated as the HCl salt. ¹HNMR (300 MHz, D₂O): δ 0.80 (t, 6H), 0.8-1.90 (m, 124H), 2.18 (b,4H), 2.80-2.95 (m, 20H), 3.05(t, 4H), 3.25(s, 6H), 3.25-4.25 (m, 74H), 4.85-5.05 (br, 7H); MS (MALDI) m/z calcd for C₁₆₀H₃₀₀N₂₆O₅₅ 3468, Found 3467.

### Example 11-8

### Preparation of Compound 87

Compound 87 was synthesized using the general procedures described in example 9 for each step as follows: coupled 3 with Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); coupled with CH₃O(CH₂CH₂O)₈CH₂COOH (procedure A); Boc deprotection (procedure C). The compound 87 was isolated as the HCl salt. ¹HNMR (300 MHz, D₂O): δ 0.80 (t, 6H), 0.8-1.90 (m, 124H), 2.18 (b, 4H), 2.5 (t, 4H), 2.80-2.95 (m, 20H), 3.05 (t, 4H), 3.25 (s, 6H), 3.25-4.25 (m, 118H), 4.85-5.05 (br, 7H); MS (MALDI) m/z calcd for C₁₈₂H₃₄₄N₂₆O₆₅ 3936, Found 3935.

### Example 11-9

### Preparation of Compound 88

Compound 88 was synthesized using the general procedures described in example 9 for each step as follows: coupled between 3 and Palmitoyl-Lys(Fmoc)-OH (procedure A); Fmoc deprotection (procedure B); further coupled with Fmoc-Lys(Boc)-Lys(Boc)-Lys(Boc)-Lys(Boc)-Lys(Boc)-OH (procedure A); Fmoc deprotection (procedure B); further coupled with Fmoc-Lys(Boc)-Lys(Boc)-Lys(Boc)-Lys(Boc)-Gly-OH (procedure A); Fmoc deprotection (procedure B); coupled with NHS-3-maleimideopropionate (procedure A); Boc deprotection (procedure C). The compound 88 was isolated as the HCl salt. ¹HNMR (300 MHz, D₂O): δ 0.80 (t, 6H), 0.8-1.90 (m, 172H), 2.18 (b,4H), 2.5 (t, 4H), 2.80-2.95 (m, 36H), 3.05 (t, 4H), 3.25-4.25 (m, 118H), 4.85-5.05 (br, 7H), 6.75(s, 4H); MS (MALDI) m/z calcd for C₂₁₂H₃₈₈N₄₆O₆₃ 4589, Found 4586.

### Example 11-10

### Preparation of Compound 89

Compound 89 was synthesized using the general procedures described in example 9 for each step as follows: coupled 3 with Palmitoyl-Lys(Fmoc)-OH (procedure A); Fmoc deprotection (procedure B); coupled with NHS-3-maleimideopropionate (procedure A); coupled with CYGRKKRRQRRR (procedure E); Boc deprotection (procedure C). The compound 89 was isolated as the HCl salt. ¹HNMR (300 MHz, D₂O): δ 0.80 (t, 6H), 0.8-1.90 (m, 200H), 2.10-2.60 (m,16), 2.80-2.95 (m, 28H), 3.05-3.20 (t, 28), 3.25-4.25 (m, 90H), 4.85-5.05 (br, 7H), 6.75 (d, 4H), 7.05 (d, 4H); MS (MALDI) m/z calcd for C₂₉₄H₅₃₂N₉₄O₈₃S₂ 6776 Found 5113 (M−1666)⁺and 1666 (M−5113)⁺.

### Example 12

### Synthesis of Oligopeptide-Cyclodextrin Conjugates 90-92

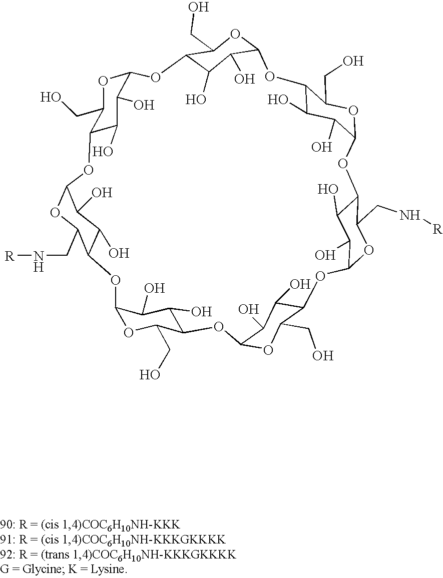

### Example 12-1

### Preparation of Compound 90

Compound 90 was synthesized using the general procedures described in example 9 for each step as follows: coupled 3 with 1,4-cis-Fmoc-NH—C₆H₁₀—COOH (procedure A);

Fmoc deprotection (procedure B); further coupled with Fmoc-Lys(Boc)-Lys(Boc)-Lys(Boc)-OH (procedure A); Fmoc deprotection (procedure B); Boc deprotection (procedure C). The compound 90 was isolated as the HCl salt. ¹HNMR (300 MHz, MeOD): δ 1.20-2.00 (m, 50H), 2.25-2.45 (m, 2H), 2.80-2.95 (m, 12H), 3.05(t, 4H), 3.25-4.40 (m, 50H), 4.85-5.05 (br, 7H), 6.75(s, 4H); MS (MALDI) m/z calcd for C₉₆H₁₆₆N₁₀O₄₁ 2152, Found 2174.

### Example 12-2

### Preparation of Compound 91

Compound 91 was synthesized using the general procedures described in example 9 for each step as follows: coupled 3 with 1,4-cis-Fmoc-NH—C₆H₁₀—COOH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 91 was isolated as the HCl salt. MS (MALDI) m/z calcd. for C₁₄₄H₂₆₈N₃₄O₅₁ 3288, Found 3310.

### Example 12-3

### Preparation of Compound 92

Compound 92 was synthesized using the general procedures described in example 9 for each step as follows: coupled 3 with 1,4-trans-Fmoc-NH—C₆H₁₀—COOH (procedure A); Fmoc deprotection (procedure B); further coupled with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 92 was isolated as the HCl salt. ¹HNMR (300 MHz, D₂O): 1.10-1.90 (m, 100H), 2.35 (br, 2H), 2.80-3.00 (m, 28H), 3.50-4.40 (m, 64), 4.75-4.90 (br, 7H); MS (MALDI) m/z calcd. for C₁₄₄H₂₆₈N₃₄O₅₁ 3288, Found 3316.

### Example 13

### Synthesis of Oligopeptide-Cyclodextrin Conjugates 93-94

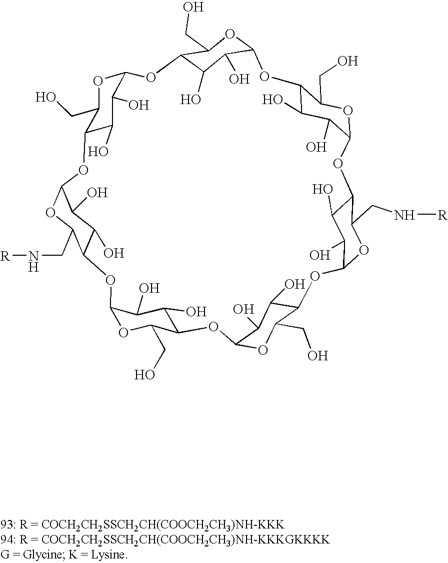

### Example 13-1

### Preparation of Compound 93

Compound 93 was synthesized using the general procedures described above in example 9 for each step as follows: coupled 3 with Boc-NHCH(CO₂Et)CH₂SSCH₂CH₂COOH (procedure A); Boc deprotection (procedure C); coupled with Boc-Lys(Boc)-Lys(Boc)-Lys(Boc)-OH (procedure A); Boc deprotection (procedure C). The compound 93 was isolated as the HCl salt. MS (MALDI) m/z calcd for C₉₄H₁₇₀N₁₆O₄₅S₄ 2372, Found 2371.

### Example 13-2

### Preparation of Compound 94

Compound 94 was synthesized using the general procedures described above in example 9 for each step as follows: coupled 3 with Boc-NHCH(CO₂Et)CH₂SSCH₂CH₂COOH (procedure A); Boc deprotection (procedure C); coupled with Fmoc-Lys(Boc)-Lys(Boc)-Lys(Boc)-OH (procedure A), Fmoc deprotection (procedure B); coupled with Boc-Lys(Boc)-Lys(Boc)-Lys(Boc)-Lys(Boc)-Gly-OH (procedure A); Boc deprotection (procedure C). The compound 94 was isolated as the HCl salt. ¹HNMR (300 MHz, D₂O): δ 1.05-1.95 (m, 90H), 2.85-2.95 (m, 28H), 3.12-4.4 (m, 70H), 4.85-4.95 (br, 7H).

### Example 14

### Synthesis of Oligopeptide-Cyclodextrin Conjugates 95-96

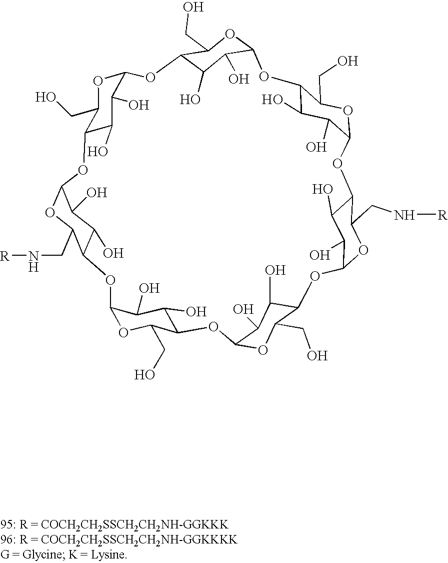

### Example 14-1

### Preparation of Compound 95

Compound 95 was synthesized using the general procedures described above in example 9 for each step as follows: coupled 3 with o-PySSCH₂CH₂COOH (procedure A); coupled with Boc-Lys(Boc)-Lys(Boc)-Lys(Boc)-Gly-Gly-NHCH₂CH₂SH (procedure A); Boc deprotection (procedure C). The compound 95 was isolated as the HCl salt. MS (MALDI) m/z calcd for C₉₆H₁₇₆N₂₀O₄₅S₄ 2451, Found 2453.

### Example 14-2

### Preparation of Compound 96

Compound 96 was synthesized using the general procedures described above in example 9 for each step as follows: coupled between 3 and o-PySSCH₂CH₂COOH (procedure A); coupled with Boc-Lys(Boc)-Lys(Boc)-Lys(Boc)-Lys(Boc)-Gly-Gly-NHCH₂CH₂SH (procedure A); Boc deprotection (procedure C). The compound 96 was isolated as the HCl salt. ¹HNMR (300 MHz, D₂O): δ 1.25-1.85 (m, 48H), 2.85-2.95 (m, 16 H), 3.10-4.4 (m, 74H), 4.85-4.95 (br, 7H); MS (MALDI) m/z calcd for C₁₀₈H₁₉₈N₂₄O₄₇S₄ 2706, Found 2709.

### Example 15

### Synthesis of Oligopeptide-Cyclodextrin Conjugates 97-98

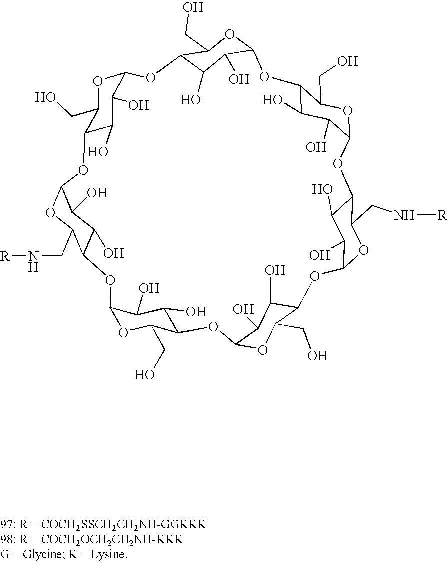

### Example 15-1

### Preparation of Compound 97

Compound 97 was synthesized using the general procedures described above in example 9 for each step as follows: coupled 3 with Fmoc-NHCH₂CH₂SSCH₂COOH (procedure A); Fmoc deprotection (procedure B); coupled with Fmoc-Lys(Boc)-Lys(Boc)-Lys(Boc)-Gly-Gly-OH (procedure A); Fmoc deprotection (procedure B); Boc deprotection (procedure C). The compound 97 was isolated as the TFA salt. ¹HNMR (300 MHz, D₂O): δ 1.25-1.85(m, 36H), 2.85-2.95(m, 12H), 3.10-4.4 (m, 60H), 4.85-4.95(br, 7H); MS (MALDI) m/z calcd for C₈₆H₁₅₈N₁₆O₄₁S₄ 2199, Found 1299 (M−920+Na)⁺ and 942 (M−1278+Na)⁺.

### Example 15-2

### Preparation of Compound 98

Compound 98 was synthesized using the general procedures described above in example 9 for each step as follows: coupled between 3 and Fmoc-CH₂CH₂OCH₂COOH (procedure A); Fmoc deprotection (procedure B); coupled with Boc-Lys(Boc)-Lys(Boc)-Lys(Boc)-OH (procedure A); c. Boc deprotection (procedure C). The compound 98 was isolated as the TFA salt. ¹HNMR (300 MHz, D₂O): δ 1.25-1.85(m, 36H), 2.85-2.95(m, 12H), 3.10-4.4 (m, 60H), 4.85-4.95(br, 7H); MS (MALDI) m/z calcd for C₈₆H₁₅₈N₁₆O₄₃ 2103, Found 2129.

### Example 16

### Synthesis of Oligopeptide-Cyclodextrin Conjugates 99

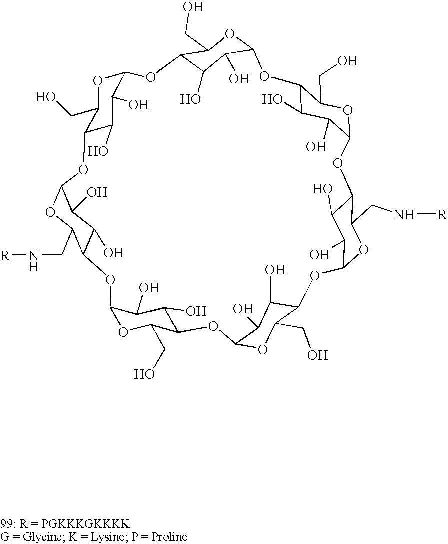

Compound 99 was synthesized using the general procedures described above in example 9 for each step as follows: coupled 3 with Fmoc-Pro-OH (procedure A); Fmoc deprotection (procedure B); coupled with Fmoc-Gly-OH (procedure A); Fmoc deprotection (procedure B); coupled with Fmoc-Lys(Boc)-Lys(Boc)-Lys(Boc)-OH (procedure A); Fmoc deprotection (procedure B); further coupled with Boc-Lys(Boc)-Lys(Boc)-Lys(Boc)-Lys(Boc)-Gly-OH (procedure A); Boc deprotection (procedure C). The compound 99 was isolated as the HCl salt. ¹HNMR (300 MHz, D₂O): δ 1.25-1.95(m, 92H), 2.85-2.95 (m, 28H), 3.0-3.15 (m, 4H), 3.20-4.4 (m, 66H), 4.85-4.95 (br, 7H); MS (MALDI) m/z calcd for C₁₄₄H₂₆₆N₃₆O₅₃ 3349, Found 3367.

### Example 17

### Synthesis of Oligopeptide-Cyclodextrin Conjugate 100

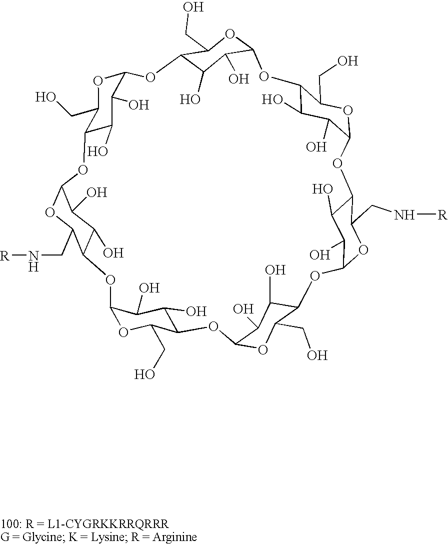

Compound 100 was synthesized as described in the above scheme using the general procedures in example 9 for each step as follows: coupled between 3 and NHS-3-maleimideopropionate (procedure A); coupled with CYGRKKRRQRRR (CTAT) (procedure F); Boc deprotection (procedure C). The compound 100 was isolated as the TFA salt. MS (MALDI) m/z calcd for C₁₉₀H₃₂₈N₇₀O₆₉S₂ 4761, Found 4767.

### Example 18

### Synthesis of Oligopeptide-Cyclodextrin Conjugate 101

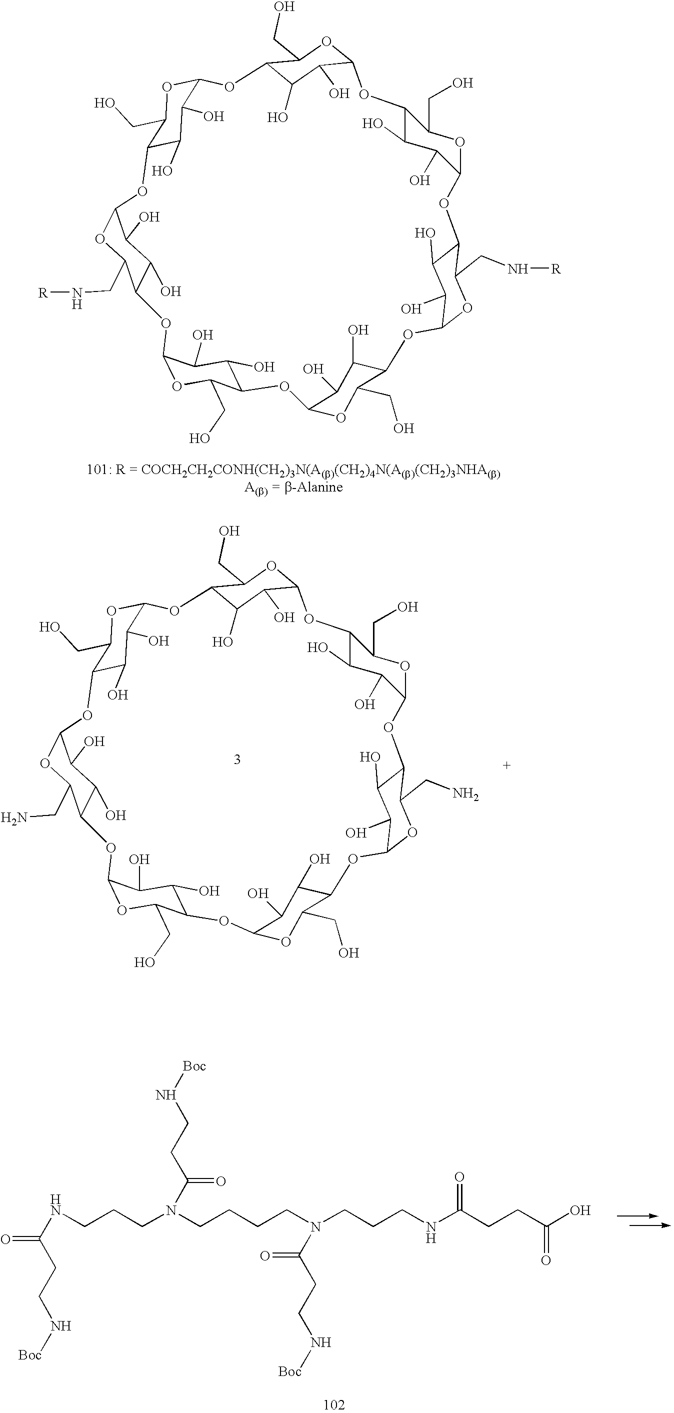

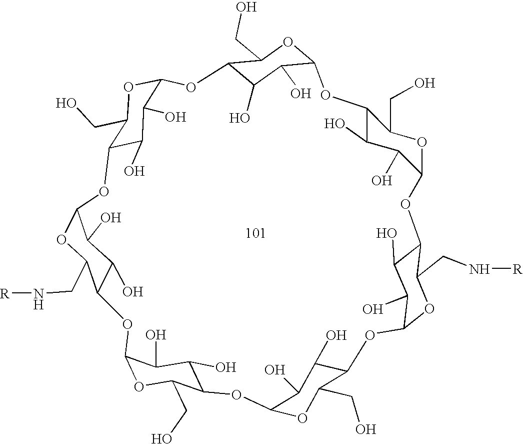

Compound 101 was synthesized using the general procedures described above in example 9 for each step as follows: coupled 3 with compound 102 (procedure A); Boc deprotection (procedure C). The compound 101 was isolated as the TFA salt. ¹HNMR (300 MHz, D₂O): δ 1.30-1.80 (m, 16H), 2.25-2.85(m, 20H), 3.0-3.90(m, 66H), 4.85-4.95(br, 7H); MS (MALDI) m/z calcd for C₈₈H₁₅₈N₁₆O₄₃ 2128, Found 2152 (M+Na)⁺.

### Example 19

### Synthesis of Oligopeptide-Cyclodextrin(alpha) Conjugates 107

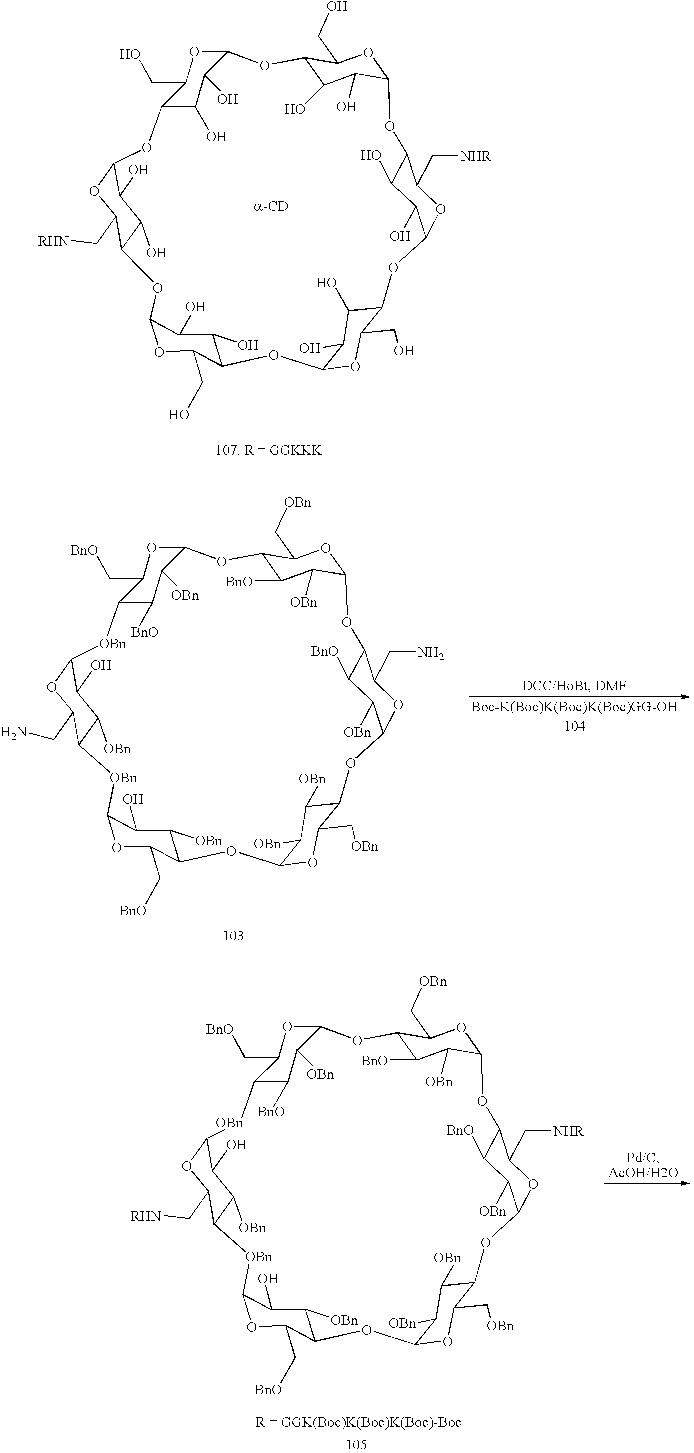

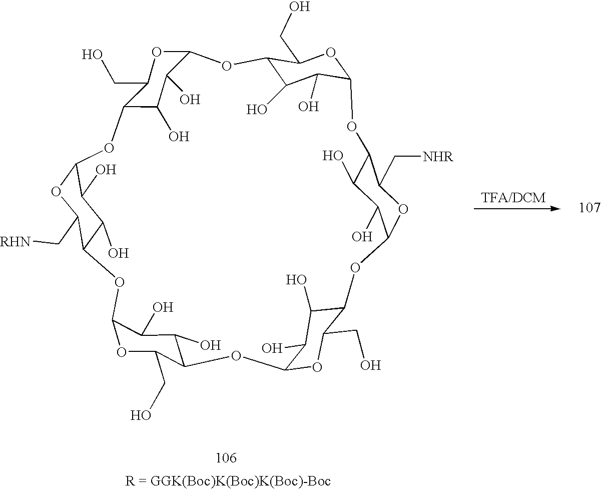

### Example 19-1

### Preparation of Compound 103

Compound 103 was prepared using the same procedure as described for the synthesis of compound 8.

### Example 19-2

### Preparation of Compound 105

To a solution of compound 103 (250 mg, 0.104 mmol. 1 eq) and Boc-Lys(Boc)-Lys(Boc)-Lys(Boc)-Gly-Gly-OH (104) (228, mg, 0.248 mmol, 2.4 eq) in DMF was added HOBt (51 mg, 0.248 mmol, 2.4 eq) followed by DCC (34 mg, 0.248 mmol, 2.4 eq). The resulting mixture was stirred at room temperature overnight and then concentrated under reduced pressure. The residue was dissolved in ethyl acetate and washed with 1 N HCl (aq), 0.1 N NaOH (aq) and brine. The organic phase was dried over MgSO₄ and then evaporated to dryness. The residue was partially dissolved in ether and filtered to remove DCU. The filtrate was concentrated and purified by silica gel column chromatography (eluents: CH₂Cl₂ to 6% MeOH/CH₂Cl₂) to give 337 mg (77%) of the desired product 105.

### Example 19-3

### Preparation of Compound 106

To a solution of compound 105 (321 mg, 0.076 mmol, 1 eq) in AcOH/H₂O (1:1) (14 mL) was added 10% Pd/C (357 mg) and Pd black (40 mg). The resulting mixture was stirred under H₂ pressure (balloon) overnight and then filtered through celitc. The filtrate was concentrated and used to the next step without further purification.

### Example 19-4

### Preparation of Compound 107

Compound 106 (211 mg, 0.076 mmol) was dissolved in a mixed solvent of TFA/DCM (3/1). The resulting solution was stirred at room temperature for 2.5 h. The reaction solution was concentrated, treated with water and then filtered through a plug of cotton. The solution was lyophilized to provide 100 mg of compound 107. ¹HNMR (300 MHz, D₂O): δ 1.25-1.85 (m, 36H), 2.80-2.95 (m, 12H), 3.40-4.25 (m 50H), 4.85-4.95 (br, 6H); MS (MALDI) m/z calcd for C₈₀H₁₄₆N₁₈O₃₈ 1968, Found 1991.

### Example 20

### Synthesis of Oligopeptide-Cyclodextrin Conjugate 108a-d

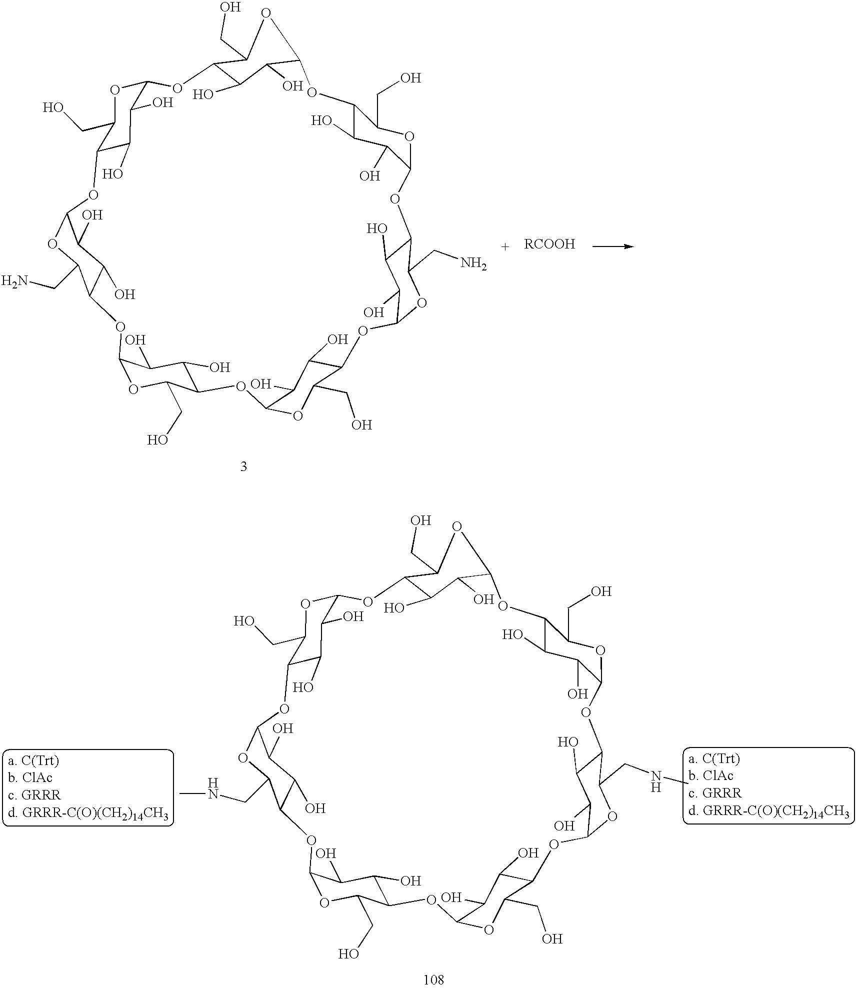

### Example 20-1

### General Procedure H-Coupling of Peptides with A,D-diaminocyclodextrines

To a solution of peptide with free carboxylic acid (2.2 eq) in DMF was added TBTU (2.2 eq.), HOBt (2.2 eq), and DIEA (4.4 eq). The mixture was stirred for 2-5 min and added to a solution of diamino functionalized cyclodextrin (1.0 eq) in DMF. The mixture was stirred for 24 h, then evaporated. The residue was suspended in water and the solid was collected by filtration and dried to give peptide-cyclodextrin conjugate.

### Example 20-2

### General Procedure I-Deprotection of Fmoc Protecting Group

Fmoc protected amino compound was dissolved in DMF/piperidine (7:3) mixture and stirred for 1-3 h until Fmoc group is completely removed (monitored by HPLC). The solvent was evaporated and the residue was suspended in diethyl ether. The precipitate was collected and dried to give amino compound.

### Example 20-3

### General Procedure J-Removal of Boc and/or Trt Groups

A Boc and/or Trt protected compound was dissolved in TFA/Et₃SiH (99:1) mixture and stirred for 1 h. The solvent was evaporated and the residue was dissolved in water and purified by HPLC.

### Example 20-4

### General Procedure K-Removal of Alloc Group

To a solution of Alloc protected amino compound in DMF/AcOH/DIEA (10:3:2) mixture was added Pd(PPh₃)₄ (0.1 eq). The mixture was purged with nitrogen and stirred under nitrogen protection for 12-24h until all Alloc groups were removed (monitored by HPLC). The solvent was evaporated and the residue was suspended in water. The precipitate was collected, washed with EtOAc and dried to give desired amino compound.

### Example 20-5

### Preparation of Compound 108a

Compound 108a was prepared following the general procedure H between diaminocyclodextrin 3 and Fmoc-Cys(Trt)-OH and the general procedure I to remove Fmoc group in 83% overall yield. ¹H-NMR (300 MHz, DMSO-d₆): δ 7.20-7.4 (m, 30H), 5.5-6.0 (m, 12H), 4.83 (s, 7H), 3.0-4.0 (m, 46H), 2.34 (m, 2H), 2.10 (m, 2H).

### Example 20-6

### Preparation of Compound 108b

To a solution of A,D-diaminocyclodextrin 3 (100 mg, 88 umol) in 5 mL of anhydrous DMF was added DIEA (52 uL), followed by chloroacetic anhydride (36 mg, 211 umol). The mixture was stirred for 4 h and evaporated to give 108b which was used without purification. MS (MALDI) m/z Calcd. For C₄₆H₇₄Cl₂N₂O₃₅ 1284, Found 1307.

### Example 20-7

### Preparation of Compound 108c

Compound 108c was prepared following the general procedure H between diaminocyclodextrin 3 and Fmoc-Arg-Arg-Arg-Gly-OH and the general procedure I to remove Fmoc group in 23% yield (purified by HPLC). MS (MALDI) m/z Calcd. For C₈₂H₁₅₀N₂₈O₄₁ 2183, Found 2184.

### Example 20-8

### Preparation of Compound 108d

Compound 108d was prepared following the general procedure H between 108c and palmitic acid and purified by HPLC. ¹H-NMR (300 MHz, D₂O): δ 4.7-5.0 (m, 7H), 3.0-4.4 (m, 64H), 1.4-1.8 (m, 24H), 1.0-1.4 (m, 62H); MS (MALDI) m/z Calcd. For C₁₁₄H₂₁₀N₂₈O₄₃ 2660, Found 2661.

### Example 21

### Synthesis of Oligopeptide-Cyclodextrin Conjugate 109

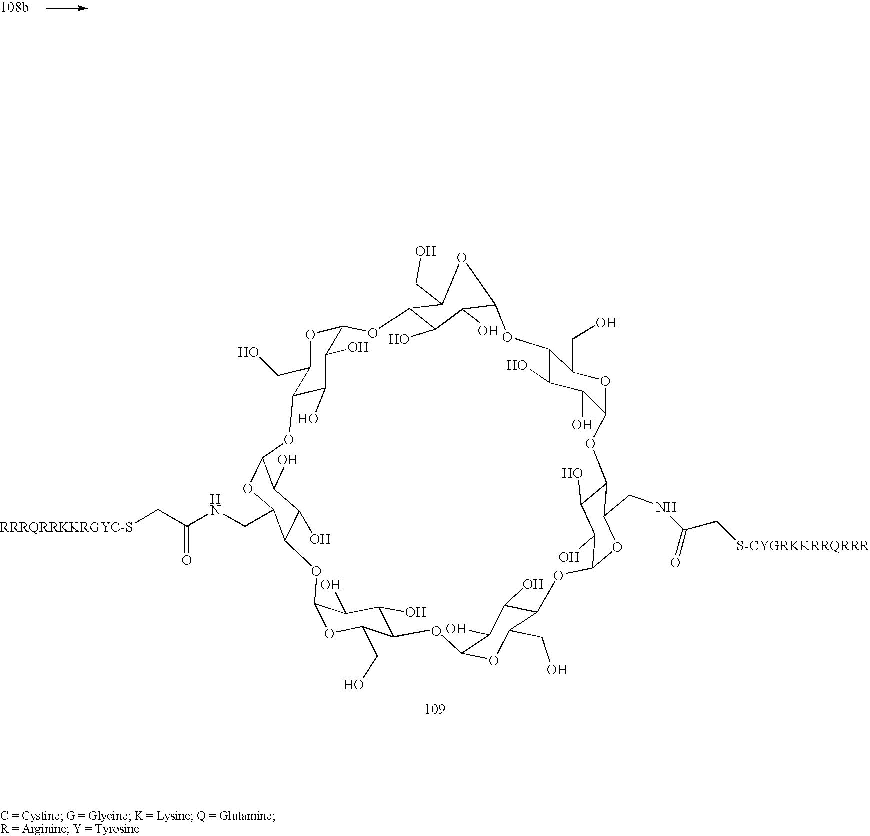

To a solution of 108b (0.96 mg, 0.75 umol) in 1 mL of 0.1M NaHCO₃ was added TAT peptide (CYGRKKRRQRRR) (2.5 mg, 1.5 umol). The mixture was purged with nitrogen and stirred under nitrogen for 3 days, then purified by HPLC to give compound 109 (0.6 mg). MS (MALDI) m/z Calcd. For C₁₈₀H₃₁₈N₆₈O₆₅S₂ 4536, Found 4537.

### Example 22

### Synthesis of Oligopeptide-Cyclodextrin Conjugate 110

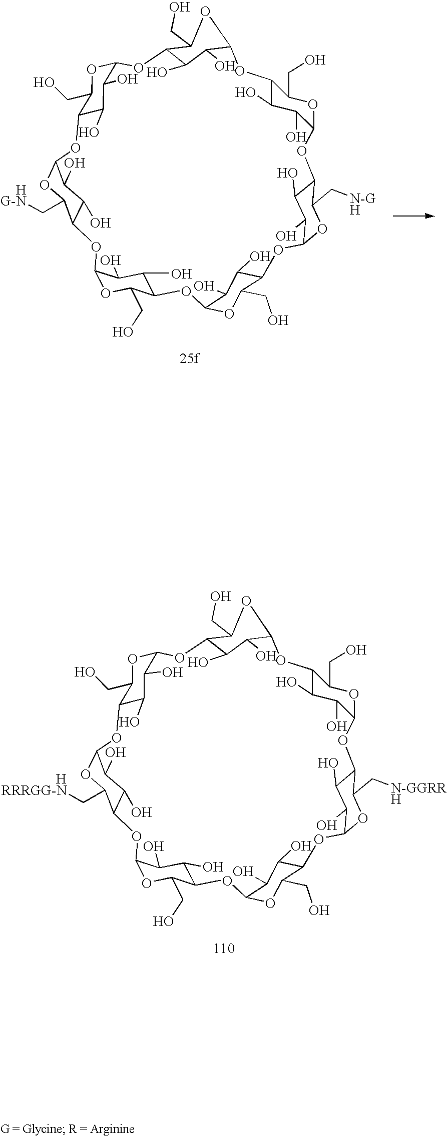

Compound 110 was prepared after HPLC purification by the general procedure H between glycinocyclodextrin 25f and Fmoc-Arg-Arg-Arg-Gly-OH and the general procedure I to remove Fmoc group. ¹H-NMR (300 MHz, D₂O): δ 4.7-5.0 (m, 7H), 4.27 (m, 4H), 3.0-4.0 (m, 74H), 1.4-2.0 (m, 24H); MS (MALDI) m/z Calcd. For C₈₆H₁₅₆N₃₀O₄₃ 2297, Found 2322.

### Example 23

### Synthesis of Oligopeptide-Cyclodextrin Conjugate 111a-j

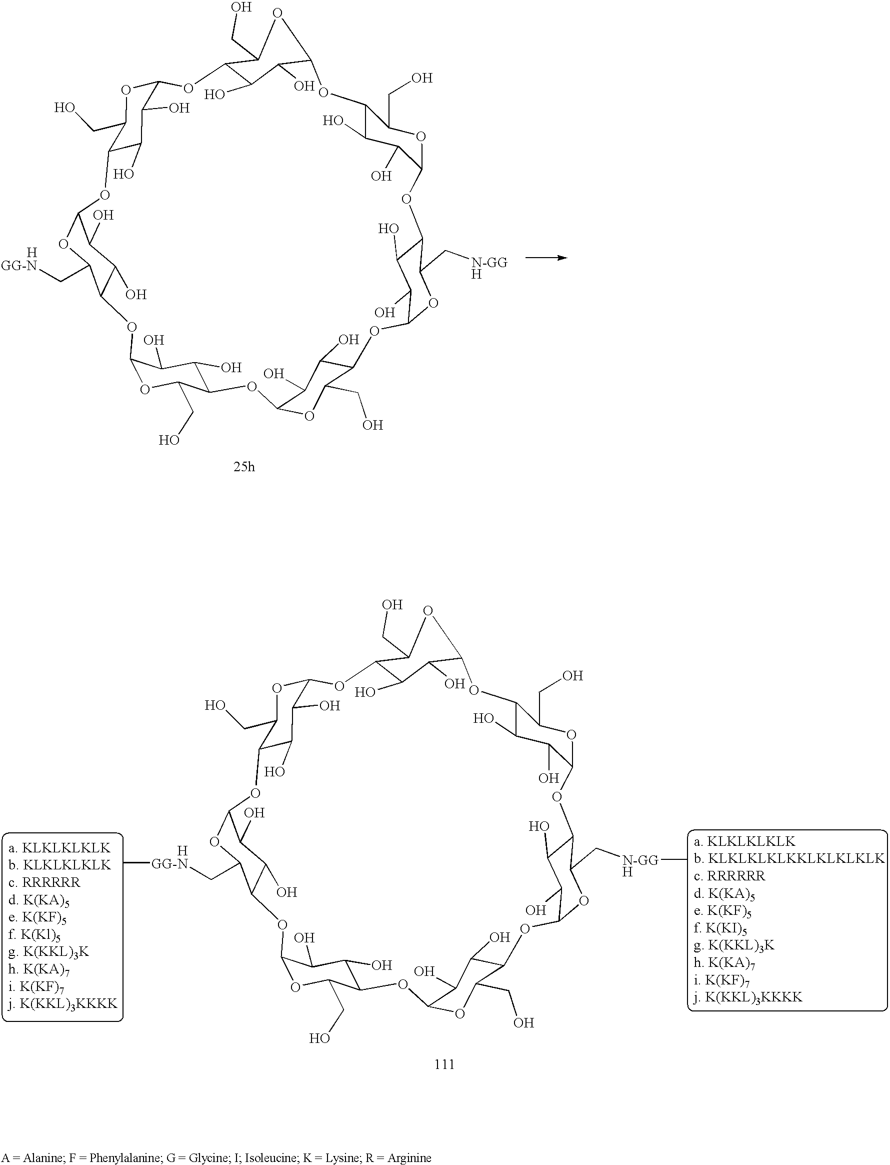

### Example 23-1

### Preparation of Compound 111a

Compound 111a was prepared after HPLC purification by the general procedure H between glycinocyclodextrin 25h and Fmoc-Lys(Boc)-Leu-Lys(Boc)-Leu-Lys(Boc)-Leu-Lys(Boc)-Leu-Lys(Boc)-OH and then the general procedure I to remove Fmoc group and the general procedure J to remove Boc group. ¹H-NMR (300 MHz, D₂O): δ 4.7-5.0 (m, 7H), 3.0-4.4 (m, 108H), 1.4-1.8 (m, 84H), 0.75 (m, 48H); MS (MALDI) m/z Calcd. For C₁₅₈H₂₉₂N₃₄O₅₅ 3548, Found 3571.

### Example 23-2

### Preparation of Compound 111b

Compound 111b was prepared after HPLC purification by the general procedure H between 111a and Lys(Boc)-Leu-Lys(Boc)-Leu-Lys(Boc)-Leu-Lys(Boc)-Leu-Lys(Boc)-OH and then the general procedures I and J to remove Fmoc and Boc protecting groups. ¹H-NMR (300 MHz, D₂O): δ 4.7-5.0 (m, 7H), 3.0-4.4 (m, 127H), 1.4-1.8 (m, 126H), 0.75 (m, 72H). MS (MALDI) m/z Calcd. For C₂₁₂H₃₉₆N₄₈O₆₄ 4641, Found 4642.

### Example 23-3

### Preparation of Compound 111c

Compound 111c was prepared after HPLC purification by the general procedure H between glycinocyclodextrin 25h and Fmoc-Arg-Arg-Arg-Arg-Arg-Arg-OH and the general procedure I to remove Fmoc group. ¹H-NMR (300 MHz, D₂O ): δ 4.7-5.0 (m, 7H), 2.4-4.4 (m, 86H), 1.4-1.8 (m, 48H).

### Example 23-4

### Preparation of Compound 111d

Compound 111d was prepared after HPLC purification by the general procedure H between glycinocyclodextrin 25h and Fmoc-Ala-Lys(Boc)-Ala-Lys(Boc)-Ala-Lys(Boc)-Ala-Lys(Boc)-Leu-Lys(Boc)-OH and the general procedures I and J to remove Fmoc and Boc protecting groups. ¹H-NMR (300 MHz, D₂O): δ 4.7-5.0 (m, 7H), 3.0-4.4 (m, 90H), 1.2-1.8 (m, 90H); MS (MALDI) m/z Calcd. For C₁₄₀H₂₅₄N₃₆O₅₇ 3355, Found 3378.

### Example 23-5

### Preparation of Compound 111e

Compound 111e was prepared after HPLC purification by the general procedure H between glycinocyclodextrin 25h and Fmoc-Phe-Lys(Boc)-Phe-Lys(Boc)-Phe-Lys(Boc)-Phe-Lys(Boc)-Phe-Lys(Boc)-OH and the general procedures I and J to remove Fmoc and Boc protecting groups. ¹H-NMR (300 MHz, D₂O): δ 6.9-7.3 (m, 50H), 4.7-5.0 (m, 7H), 3.0-4.4 (m, 110H), 1.2-1.8 (m, 60H); MS (MALDI) m/z Calcd. For C₂₀₀H₂₉₄N₃₆O₅₇ 4114, Found 4137.

### Example 23-6

### Preparation of Compound 111f

Compound 1111 was prepared after HPLC purification by the general procedure H between glycinocyclodextrin 25h and Fmoc-Ile-Lys(Boc)-Ile-Lys(Boc)-Ile-Lys(Boc)-Ile-Lys(Boc)-Ile-Lys(Boc)-OH and the general procedures I and J to remove Fmoc and Boc protecting groups. ¹H-NMR (300 MHz, D₂O): δ 4.7-5.0 (m, 7H), 3.0-4.4 (m, 90H), 1.2-1.8 (m, 150H); MS m/z Calcd. For C₁₇₀H₃₁₄N₃₆O₅₇ 3772, Found 3773.

### Example 23-7

### Preparation of Compound 111g

Compound 111g was prepared after HPLC purification by the general procedure H between glycinocyclodextrin 25h and Fmoc-Lys(Boc)-Leu-Lys(Boc)-Lys(Boc)-Leu-Lys(Boc)-Lys(Boc)-Leu-Lys(Boc)-Lys(Boc)-OH and the general procedure I and J to remove Fmoc and Boc protecting groups. ¹H-NMR (300 MHz, D₂O): δ 4.7-5.0 (m, 7H), 3.0-4.4 (m, 98H), 1.4-1.8 (m, 102H), 0.75 (m, 36H); MS (MALDI) m/z Calcd. For C₁₇₀H₃₁₈N₄₀O₅₇ 3835, Found 3836.

### Example 23-8

### Preparation of Compound 111h

Compound 111h was prepared after HPLC purification by the general procedure H between glycinocyclodextrin 25h and Fmoc-Ala-Lys(Boc)-Ala-Lys(Boc)-Ala-Lys(Boc)-Ala-Lys(Boc)-Ala-Lys(Boc)-Ala-Lys(Boc)-Ala-Lys(Boc)-OH and the general procedure I and J to remove Fmoc and Boc protecting groups. ¹H-NMR (300 MHz, D₂O): δ 4.7-5.0 (m, 7H), 3.0-4.4 (m, 106H), 1.2-1.8 (m, 126H); MS (MALDI) m/z Calcd. For C₁₇₆H₃₂₂N₄₈O₆₅ 4151, Found 4152.

### Example 23-9

### Preparation of Compound 111i

Compound 111i was prepared after HPLC purification by the general procedure H between glycinocyclodextrin 25h and Fmoc-Phe-Lys(Boc)-Phe-Lys(Boc)-Phe-Lys(Boc)-Phe-Lys(Boc)-Phe-Lys(Boc)-Phe-Lys(Boc)-Phe-Lys(Boc)-OH and the general procedures I and J to remove Fmoc and Boc protecting groups. ¹H-NMR (300 MHz, D₂O): δ 6.9-7.3 (m, 70H), 4.7-5.0 (m, 7H), 3.0-4.4 (m, 134H), 1.2-1.8 (m, 84H); MS (MALDI) m/z Calcd. For C₂₆₀H₃₇₈N₄₈O₆₅ 5215, Found 5238.

### Example 23-10

### Preparation of Compound 111j

Compound 111j was prepared after HPLC purification by the general procedure H between glycinocyclodextrin 25h and Fmoc-Lys(Boc)-Lys(Boc)-Lys(Boc)-Lys(Boc)-Lys(Leu-Lys(Boc)-Lys(Boc)-Leu-Lys(Boc)-Lys(Boc)-Leu-Lys(Boc)-Lys(Boc))-OH and the general procedure I and J to remove Fmoc and Boc protecting groups. ¹H-NMR (300 MHz, D₂O): δ 4.7-5.0 (m, 7H), 3.0-4.4 (m, 116H), 1.4-1.8 (m, 138H), 0.75 (m, 36H); MS (MALDI) m/z Calcd. C₂₀₆H₃₉₀N₅₂O₆₃ 4600, Found 4623.

### Example 24

### Synthesis of Oligopeptide-Cyclodextrin Conjugate 112

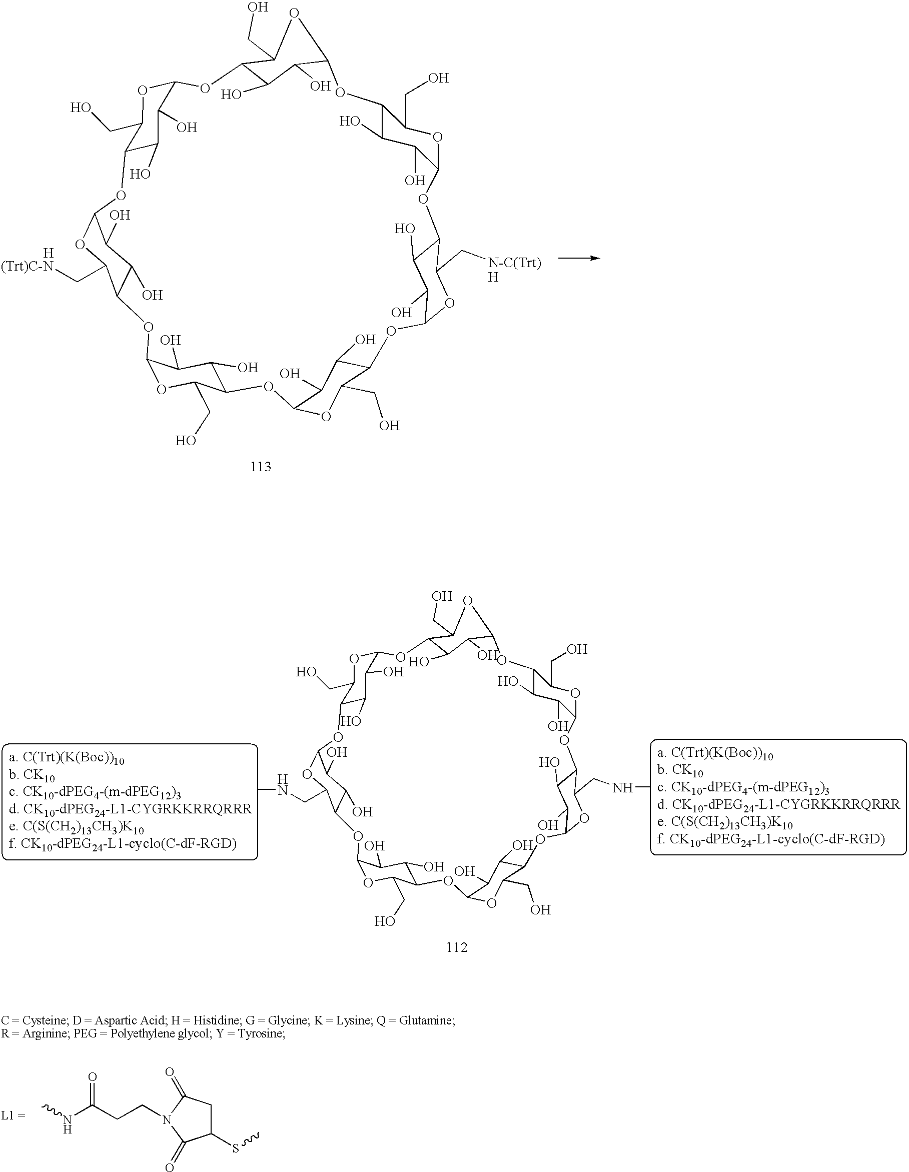

### Example 24-1

### Preparation of Compound 112a

Cycteinocyclodextrin 113 was coupled with Fmoc-Lys(Boc)-Lys(Boc)-Lys(Boc)-Lys(Boc)-Lys(Boc)-OH following the general procedure H. Fmoc group of the resulted intermediate was removed under the general procedure I and the free amine was coupled with Fmoc-Lys(Boc)-Lys(Boc)-Lys(Boc)-Lys(Boc)-Lys(Boc)-OH following the general procedure H. Removal of Fmoc protecting group was accomplished following the general procedure I to give compound 112a.

### Example 24-2

### Preparation of Compound 112b

Compound 112a was subject to the general procedure J to remove Boc and Trt groups. The resulting mixture was purified by HPLC to give compound 112b. ¹H-NMR (300 MHz, D₂O): δ 4.7-5.0 (m, 7H), 2.7-4.4 (m, 108H), 1.2-1.8 (m, 120H); MS (MALDI) m/z Calcd. For C₁₆₈H₃₂₂N₄₄O₅₅S₂ 3900, Found 3901.

### Example 24-3

### Preparation of Compound 112c

To a solution of compound 112a (10 mg, 1.57 umol) in 1 mL of DMF was added DIEA (1.2 uL) and NHS-dPEG₄-(m-dPEG₁₂)₃ ester (Quanta) (15.5 mg, 6.4 umol). The mixture was stirred for 2 days and the solvent was removed under reduced pressure to give a crude intermediate. The intermediate was subject to the general procedure J to remove Boc and Trt groups. The resulting product was purified by HPLC to give compound 112c. ¹H-NMR (300 MHz, D₂O): δ 4.7-5.0 (m, 7H), 3.6 (m, 390H), 2.7-4.4 (m, 108H), 1.2-1.8 (m, 124H); MS (MALDI) m/z Calcd. For C₃₇₆H₇₂₄N₅₄O₁₅₃S₂ 8509, Found 8510.

### Example 24-4

### Preparation of Compound 112d

To a solution of compound 112a (1 eq, 1 umol) in 0.25 mL of DMF was added DIEA (3 eq) and NHS-dPEG₂₄-MAL (Quanta) (3 eq). The mixture was stirred for 2 days at room temperature. The reaction mixture was diluted with 0.5 mL of phosphate buffer (50 mM NaHPO₄, 10 mM EDTA, pH 7.2) and 0.4 mL of MeOH and then the TAT peptide (CYGRKKRRQRRR) (3 eq) was added. The resulting mixture was purged with nitrogen and stirred under nitrogen for 2 days. The solvent was removed under reduced pressure and the crude residue was washed with water (2×1 mL). The crude residue was subject to the general procedure J to remove Bee and Trt groups. The resulting product was purified by HPLC to give compound 112d. ¹H-NMR (300 MHz, D₂O): δ 7.19 (d, 4H), 6.75 (d, 4H), 4.7-5.0 (m, 7H), 3.6 (m, 200H), 2.7-4.4 (m, 192H), 1.2-1.8 (m, 196H); MS (MALDI) m/z Calcd. For C₄₁₈H₇₈₀N₁₁₄O₁₄₁S₄ 9781, Found 9782.

### Example 24-5

### Preparation of Compound 112e

To a solution of compound 112b (1 eq, 1.5 mg, 0.32 umol) in 0.2 mL of EtOH, 0.1 mL of H₂O, and 20 uL of AcOH was added followed by addition of 2-(tetradecyldisulfanyl)pyridine (4eq, 0.43 mg, 1.28 umol) in 0.1 mL of EtOH. The reaction mixture was stirred for 1 day at room temperature. The solvent was removed and the residue was purified by HPLC to give compound 112e. MS (MALDI) m/z Calcd. For C₁₉₆H₃₇₈N₄₄O₅₅S₄ 4356, Found 4358.

### Example 24-6

### Preparation of Compound 112f

To a solution of compound 112a (1 eq, 1 umol) in 0.25 mL of DMF, DIEA (3 eq) and NHS-dPEG₂₄-MAL (Quanta) (3 eq) was added. The reaction mixture was stirred for 2 days at room temperature. The reaction mixture was diluted with 0.2 mL of phosphate buffer (50 mM NaHPO₄, 10 mM EDTA, and pH 7.2) and 0.5 mL of MeOH and then cyclo(C-dF-RGD) peptide (4 eq) was added. The resulting mixture was purged with nitrogen and stirred under nitrogen for 2 days. The solvent was removed under reduced pressure and the residue was washed with water (2×1 mL). The residue was subject to the general procedure C to remove Boc and Trt groups. The resulting product was purified by HPLC to give compound 112f. ¹H-NMR (300 MHz, D₂O): δ 7.3 (m, 10H), 4.7-5.0 (m, 7H), 3.6 (m, 200H), 2.7-4.4 (m, 142H), 1.2-1.8 (m, 128H).

### Example 25

### Synthesis of Oligopeptide-cyclodextrin Conjugate 114a-u

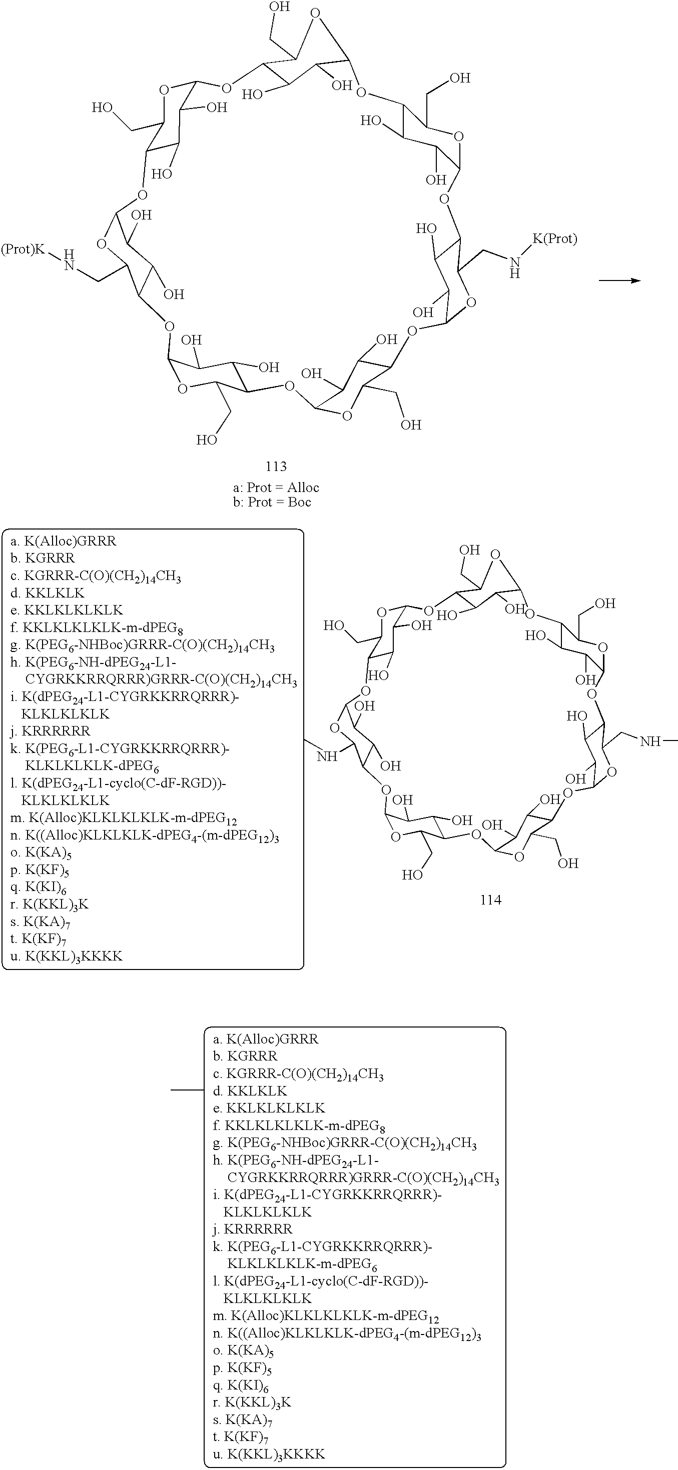

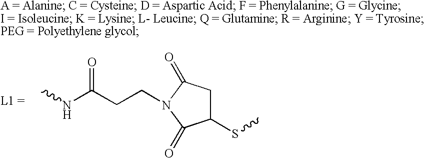

### Example 25-1

### Preparation of Compound 114a

Compound 114a was prepared after HPLC purification by the general procedure H between cyclodextrin 113a and Fmoc-Arg-Arg-Arg-Gly-OH and the general procedures and J to remove Fmoc and Boc protecting groups. ¹-NMR (300 MHz, D₂O): δ 5.8 (m, 2H), 5.1 (m, 4H), 4.7-5.0 (m, 7H), 2.7-4.4 (m, 74H), 1.2-1.8 (m, 36H); MS (MALDI) m/z Calcd. For C₁₀₂H₁₇₄N₃₂O₄₇ 2608, Found 2609.

### Example 25-2

### Preparation of Compound 114b

Compound 114a was subject to the general procedure K for the removal of Alloc groups to give compound 114b. ¹H-NMR (300 MHz, D₂O): δ 4.7-5.0 (m, 7H), 2.7-4.4 (m, 70H), 1.2-1.8 (m, 36H); MS (MALDI) m/z Calcd. For C₉₄H₁₇₄N₃₂O₄₃ 2440, Found 2441.

### Example 25-3

### Preparation of Compound 114c

Compound 114c was prepared after HPLC purification by first subject to the general procedure H between 114a and palmitic acid and the general procedure K to remove Alloc groups. ¹H-NMR (300 MHz, D₂O): δ 4.7-5.0 (m, 7H), 2.7-4.4 (m, 74H), 1.2-1.8 (m, 36H), 0.75-1.25 (m, 32H); MS (MALDI) m/z Calcd. For C₁₂₆H₂₃₄N₃₂O₄₅ 2916, Found 2917.

### Example 25-4

### Preparation of Compound 114d

Compound 114d was prepared after HPLC purification by the general procedure H between cyclodextrin 113a and Fmoc-Lys(Boc)-Leu-Lys(Boc)-Leu-Lys(Boc)-OH, and the general procedures K and I to remove Alloc and Fmoc protecting groups. ¹H-NMR (300 MHz, D₂O): δ 4.7-5.0 (m, 7H), 2.7-4.4 (m, 70H), 1.2-1.8 (m, 60H), 0.75-1.00 (m, 24H); MS (MALDI) m/z Calcd. For C₁₁₄H₂₁₂N₂₂O₄₅ 2610, Found 2611.

### Example 25-5

### Preparation of Compound 1114e

Compound 114e was prepared after HPLC purification by the general procedure H between cyclodextrin 114a and Fmoc-Lys(Boc)-Leu-Lys(Boc)-Leu-Lys(Boc)-Leu-Lys(Boc)-Leu-Lys(Boc)-OH and the general procedures K and I to remove Alloc and Fmoc protecting groups. ¹H-NMR (300 MHz, D₂O): δ 4.7-5.0 (m, 7H), 2.7-4.4 (m, 86H), 1.2-1.8 (m, 84H), 0.75-1.00 (m, 48H); MS (MALDI) m/z Calcd. For C₁₆₂H₃₀₄N₃₄O₅₃ 3574, Found 3597.

### Example 25-6

### Preparation of Compound 114f

Following the general procedure H, cyclodextrin 113a was coupled with Fmoc-Lys(Boc)-Leu-Lys(Boc)-Leu-Lys(Boc)-Leu-Lys(Boc)-Leu-Lys(Boc)-OH. The resulting product was then subject to the general procedure I to remove Fmoc group. The resulting product was dissolved in DMF and DIEA (3 eq) and NHS-dPEG₈ ester (Quanta) (3 eq) was added. The reaction mixture was stirred for 2 days at room temperature. Removal of the solvent gave the crude residue which was subject to the general procedures K and J to remove Alloc and Boc groups. The crude product was purified by HPLC to give compound 114f. ¹H-NMR (300 MHz, D₂O): δ 4.7-5.0 (m, 7H), 2.7-4.4 (m, 86H), 3.25 (m, 70H), 1.2-1.8 (m, 84H), 0.75-1.00 (m, 48H); MS (MALDI) m/z Calcd. For C₁₉₈H₃₇₂N₃₄O₇₁ 4363, Found 4365.

### Example 25-7

### Preparation of Compound 114g

Compound 114g was prepared after HPLC purification following the general procedure H between compound 114c and BocNH-dPEG₆-COOH. ¹H-NMR (300 MHz, D₂O): δ 4.7-5.0 (m, 7H), 2.7-4.4 (m, 74H), 3.6 (m, 28H), 1.2-1.8 (m, 42H), 0.75-1.25 (m, 32H).

### Example 25-8

### Preparation of Compound 114h

Compound 114 g (1 eq, 0.53 umol) was subject to the the general procedure J to remove Boc group. After removal of the solvent, the crude residue was dissolved in 1 mL of DMF and was added DIEA (10 eq) and NHS-dPEG₂₄-MAL (Quanta) (4 eq). The reaction mixture was stirred at room temperature for 12 h. The solvent of the reaction mixture was removed and the residue was redissolved in 1.0 mL of phosphate buffer (50 mM NaHPO₄, 10 mM EDTA, and pH 7.2), 0.5 mL of MeOH and 0.5 mL of ACN. To the resulting solution, the TAT peptide (CYGRKKRRQRRR) (4 eq) was added and the reaction mixture was purged with nitrogen and stirred under nitrogen for 2 days. The solvent was removed under reduced pressure and the crude was purified by HPLC to give compound 114h. ¹H-NMR (300 MHz, D₂O): δ 7.19 (d, 4H), 6.75 (d, 4H), 4.7-5.0 (m, 7H), 3.6 (m, 264H), 2.7-4.4 (m, 106H), 1.2-1.8 (m, 112H), 0.75-1.25 (m, 32H); MS m/z Calcd. For C₄₀₆H₇₅₀N₁₀₄O₁₄₅S₂ 9467, Found 9468.

### Example 25-9

### Preparation of Compound 114i

Cyclodextrin 113a was coupled with Fmoc-Lys(Boc)-Leu-Lys(Boc)-Leu-Lys(Boc)-Leu-Lys(Boc)-Leu-Lys(Boc)-OH following the general procedure H. To the resulting product in DMF (1.0 mL), Pd(PPh₃)₄ (0.1 eq) and Me₂NH BH₃ complex (2.2 eq) were added to remove the protected groups. The reaction mixture was purged with nitrogen and stirred under nitrogen for 2 d until all Alloc and Fmoc groups were removed (monitored by HPLC). The solvent was then evaporated and the residue was suspended in water. The aqueous suspension was washed with ether (3×0.5 mL) and lyophilized to give crude amino compound. To a solution of the curde amino compound in 0.5 mL of DMF, DIEA (10 eq) and NHS-dPEG₂₄-MAL (Quanta) (6 eq) were added. The reaction mixture was stirred for 12 h. The reaction mixture was then diluted with 0.5 mL of phosphate buffer (50 mM NaHPO₄, 10 mM EDTA, pH 7.2) and 0.2 mL of MeOH. To the resulting solution, the TAT peptide (CYGRKKRRQRRR) (6 eq) was added and the mixture was purged with nitrogen and stirred for 2 days under nitrogen. The solvent was evaporated under reduced pressure and the crude residue was washed with water (2×1 mL). The resulting intermediate was subject to the general procedure J to remove Boc protecting groups. After evaporation of solvent the residue was purified by HPLC to give compound 114i. ¹H-NMR (300 MHz, D₂O): δ 7.19 (d, 4H), 6.75 (d, 4H), 4.7-5.0 (m, 7H), 3.6 (m, 208), 2.7-4.4 (m, 162H), 1.2-1.8 (m, 172H), 0.75 (m, 48H); MS (MALDI) m/z Calcd. For C412H762N104O139S2 9455, Found 9456.

### Example 25-10

### Preparation of compound 114j

Compound 114j was prepared after HPLC purification by the general procedure H between cyclodextrin 113b and Fmoc-Arg-Arg-Arg-Arg-Arg-Arg-OH and the general procedures I and J to remove Fmoc and Boc protecting groups. ¹H-NMR (300 MHz, D₂O): δ 4.7-5.0 (m, 7H), 2.7-4.4 (m, 126H), 1.2-1.8 (m, 60H).

### Example 25-11

### Preparation of Compound 114k

Cyclodextrin 113a was coupled with Fmoc-Lys(Boc)-Leu-Lys(Boc)-Leu-Lys(Boc)-Leu-Lys(Boc)-Leu-Lys(Boc)-OH following the general procedure H. Fmoc group of the resulting compound was removed following the general procedure I. To a solution of the resulting free amine in DMF, DIEA (3 eq) and NHS-dPEG₈ ester (Quanta) (3 eq) were added and the reaction mixture was stirred for 2 days. The solvent of the reaction mixture was evaporated under reduced pressure to give a crude intermediate which was subject to the general procedure K to remove Alloc group. The crude residue was dissolved in 1 mL of DMF and coupled with NHS-dPEG₈-MAL (Thermo) (4 eq). The reaction mixture was stirred for 2 days and then the solvent of the reaction mixture was evaporated and redissolved in 0.4 mL of phosphate buffer (50 mM NaHPO₄, 10 mM EDTA, pH 7.2) and 0.7 mL of MeOH. To the solution, the TAT peptide (CYGRKKRRQRRR) (4 eq) was added, and the resulting mixture was purged with nitrogen and stirred under nitrogen for 3 days. The solvent was evaporated under reduced pressure and the residue was subject to the general procedure C to remove Boc protecting group. The crude was purified by HPLC to give compound 114k. ¹H-NMR (300 MHz, D₂O): δ 7.2 (d, 4H), 6.8 (d, 4H), 4.7-5.0 (m, 7H), 2.7-4.4 (m, 162H), 3.6 (m, 150H), 1.2-1.8 (m, 172H), 0.75-1.00 (m, 48H).

### Example 25-12

### Preparation of Compound 114l

Cyclodextrin 113a was coupled with Fmoc-Lys(Boc)-Leu-Lys(Boc)-Leu-Lys(Boc)-Leu-Lys(Boc)-Leu-Lys(Boc)-OH following the general procedure H. To a solution of the resulting product in DMF (1.0 mL), Pd(PPh₃)₄ (0.1 eq) and Me₂NH BH₃ complex (2.2 eq) were added. The reaction mixture was purged with nitrogen and stirred under nitrogen for 2 days until all Alloc and Fmoc groups were removed (monitored by HPLC). The solvent was removed and the residue was suspended in water. The aqueous suspension was washed with ether (3×0.5 mL) and lyophilized to give crude amino compound. To a solution of the crude amino compound in 0.5 mL of DMF, DIEA (10 eq) and NHS-dPEG₂₄-MAL (Quanta) (6 eq) were added and the reaction mixture was stirred for 12 h. The reaction mixture was diluted with 0.2 mL of phosphate buffer (50 mM NaHPO₄, 10 mM EDTA, and pH 7.2) and 0.5 mL of MeOH and cyclo(C-dF-RGD) peptide (7 eq) was added. The reaction mixture was purged with nitrogen and stirred for 2 days under nitrogen. The solvent was evaporated under reduced pressure and the residue was washed with water (2×1 mL) and then subject to the general procedure J to remove Boc groups. After removal of the solvent, the residue was purified by HPLC to give compound 114l. ¹H-NMR (300 MHz, D₂O): δ 7.2 (m, 10H), 4.7-5.0 (m, 7H), 3.6 (m, 208), 2.7-4.4 (m, 120H), 1.2-1.8 (m, 104H), 0.75 (m, 48H); MS (MALDI) m/z Calcd. For C412H762N104O139S2 9455, Found 9456.

### Example 25-13

### Preparation of Compound 114m

Cyclodextrin 113a was coupled with Fmoc-Lys(Boc)-Leu-Lys(Boc)-Leu-Lys(Boc)-Leu-Lys(Boc)-Leu-Lys(Boc)-OH following the general procedure H. The resulting intermediate was subject to the general procedure I to remove Fmoc group. To a solution of the resulting product in DMF, DIEA (3 eq) and NHS-dPEG₁₂ ester (Quanta) (3 eq) were added and the reaction mixture was stirred for 2 days. The solvent was evaporated under reduced pressure to give crude residue which was subject to the general procedure J to remove Boc. The crude mixture was purified by HPLC to give compound 114m. ¹H-NMR (300 MHz, D₂O): δ 5.9 (m, 2H), 5.2 (m, 4H), 4.7-5.0 (m, 7H), 2.7-4.4 (m, 90H), 3.5 (m, 102H), 1.2-1.8 (m, 84H), 0.75-1.00 (m, 48H); MS (MALDI) m/z Calcd. For C₂₂₂H₄₁₂N₃₄O₈₃ 4885, Found 4886.

### Example 25-14

### Preparation of Compound 114n

Cyclodextrin 113a was coupled with Fmoc-Lys(Boc)-Leu-Lys(Boc)-Leu-Lys(Boc)-Leu-Lys(Boc)-Leu-Lys(Boc)-OH following the general procedure H. The resulting product was subject to the general procedure I to remove Fmoc group. To a solution of the resulting product in DMF, DIEA (3 eq) and NHS-dPEG₄-(m-dPEG₁₂)₃ ester (Quanta) were added and the mixture was stirred for 2 days. The solvent was evaporated under reduced pressure to give crude residue which was subject to the general procedure J to remove Boc. The crude was purified by HPLC to give compound 114n. ¹H-NMR (300 MHz, D₂O): δ 5.9 (m, 2H), 5.2 (m, 4H), 4.7-5.0 (m, 7H), 2.7-4.4 (m, 90H), 3.6 (m, 390H), 1.2-1.8 (m, 84H), 0.75-1.00 (m, 48H); MS (MALDI) m/z Calcd. For C₃₇₉H₇₁₅N₄₃O₁₅₅ 8554, Found 8555.

### Example 25-15

### Preparation of Compound 114o

Compound 114o was prepared after HPLC purification by the general procedure H between cyclodextrin 113b and Fmoc-Ala-Lys(Boc)-Ala-Lys(Boc)-Ala-Lys(Boc)-Ala-Lys(Boc)-Ala-Lys(Boc)-OH and the general procedures I and J to remove Fmoc and Boc groups. ¹H-NMR (300 MHz, D₂O): δ 4.7-5.0 (m, 7H), 3.0-4.4 (m, 88H), 1.2-1.8 (m, 102H); MS (MALDI) m/z Calcd. For C₁₄₄H₂₆₆N₃₆O₅₅ 3382, Found 3406.

### Example 25-16

### Preparation of Compound 114p

Compound 114p was prepared after HPLC purification by the general procedure H between cyclodextrin 113b and Fmoc-Phe-Lys(Boc)-Phe-Lys(Boc)-Phe-Lys(Boc)-Phe-Lys(Boc)-Phe-Lys(Boc)-OH and the general procedures I and J to remove Fmoc and Boc groups. ¹H-NMR (300 MHz, D₂O): δ 6.9-7.3 (m, 50H), 4.7-5.0 (m, 7H), 3.0-4.4 (m, 108H), 1.2-1.8 (m, 72H); MS (MALDI) m/z Calcd. For C₂₀₄H₃₀₆N₃₆O₅₅ 4143, Found 4166.

### Example 25-17

### Preparation of Compound 114q

Compound 114q was prepared after HPLC purification by the general procedure H between cyclodextrin 113b and Fmoc-Ile Lys(Boc)-Ile Lys(Boc)-Ile Lys(Boc)-Ile Lys(Boc)-Ile Lys(Boc)-OH and the general procedures I and J to remove Fmoc and Boc groups. ¹H-NMR (300 MHz, D₂O): δ 4.7-5.0 (m, 7H), 3.0-4.4 (m, 88H), 1.2-1.8 (m, 162H); MS (MALDI) m/z Calcd. For C₁₇₄H₃₂₆N₃₆O₅₅ 3800, Found 3823.

### Example 25-18

### Preparation of Compound 114r

Compound 114r was prepared after HPLC purification by the general procedure H between cyclodextrin 113b and Fmoc-Lys(Boc)-Leu-Lys(Boc)-Lys(Boc)-Leu-Lys(Boc)-Lys(Boc)-Leu-Lys(Boc)-Lys(Boc)-OH and the general procedures I and J to remove Fmoc and Boc groups. ¹H-NMR (300 MHz, D₂O): δ 4.7-5.0 (m, 7H), 3.0-4.4 (m, 96H), 1.4-1.8 (m, 114H), 0.75 (m, 36H); MS (MALDI) m/z Calcd, For C₁₇₄H₃₃₀N₄₀O₅₅ 3862, Found 3863.

### Example 25-19

### Preparation of Compound 114s

Compound 114s was prepared after HPLC purification by the general procedure H between cyclodextrin 113b and Fmoc-Ala-Lys(Boc)-Ala-Lys(Boc)-Ala-Lys(Boc)-Ala-Lys(Boc)-Ala-Lys(Boc)-Ala-Lys(Boc)-Ala-Lys(Boc)-OH and the general procedures I and J to remove Fmoc and Boc groups. ¹H-NMR (300 MHz, D₂O): δ 4.7-5.0 (m, 7H), 3.0-4.4 (m, 104H), 1.2-1.8 (m, 138H); MS (MALDI) m/z Calcd. For C₁₈₀H₃₃₄N₄₈O₆₃ 4179, Found 4180.

### Example 25-20

### Preparation of Compound 114t

Compound 114t was prepared after HPLC purification by the general procedure H between cyclodextrin 113b and Fmoc-Phe-Lys(Boc)-Phe-Lys(Boc)-Phe-Lys(Boc)-Phe-Lys(Boc)-Phe-Lys(Boc)-Phe-Lys(Boc)-Phe-Lys(Boc)-OH and the general procedures I and J to remove Fmoc and Boc groups. ¹H-NMR (300 MHz, D₂O): δ 6.9-7.3 (m, 70H), 4.7-5.0 (m, 7H), 3.0-4.4 (m, 132H), 1.2-1.8 (m, 96H); MS (MALDI) m/z Calcd. For C₂₆₄H₃₉₀N₄₈O₆₃ 5241, Found 5264.

### Example 25-21

### Preparation of Compound 114u

Compound 114u was prepared after HPLC purification by the general procedure H between cyclodextrin 113b and Fmoc-Lys(Boc)-Lys(Boc)-Lys(Boc)-Lys(Boc)-Leu-Lys(Boc)-Lys(Boc)-Leu-Lys(Boc)-Lys(Boc)-Leu-Lys(Boc)-Lys(Boc)-OH and the general procedures I and J to remove Fmoc and Boc groups. ¹H-NMR (300 MHz, D₂O): δ 4.7-5.0 (m, 7H), 3.0-4.4 (m, 114H), 1.4-1.8 (m, 150H), 0.75 (m, 36H); MS (MALDI) m/z Calcd. C₂₁₀H₄₀₂N₅₂O₆₁ 4629, Found 4651.

### Example 26

### Synthesis of Oligopeptide-Cyclodextrin Conjugate 116

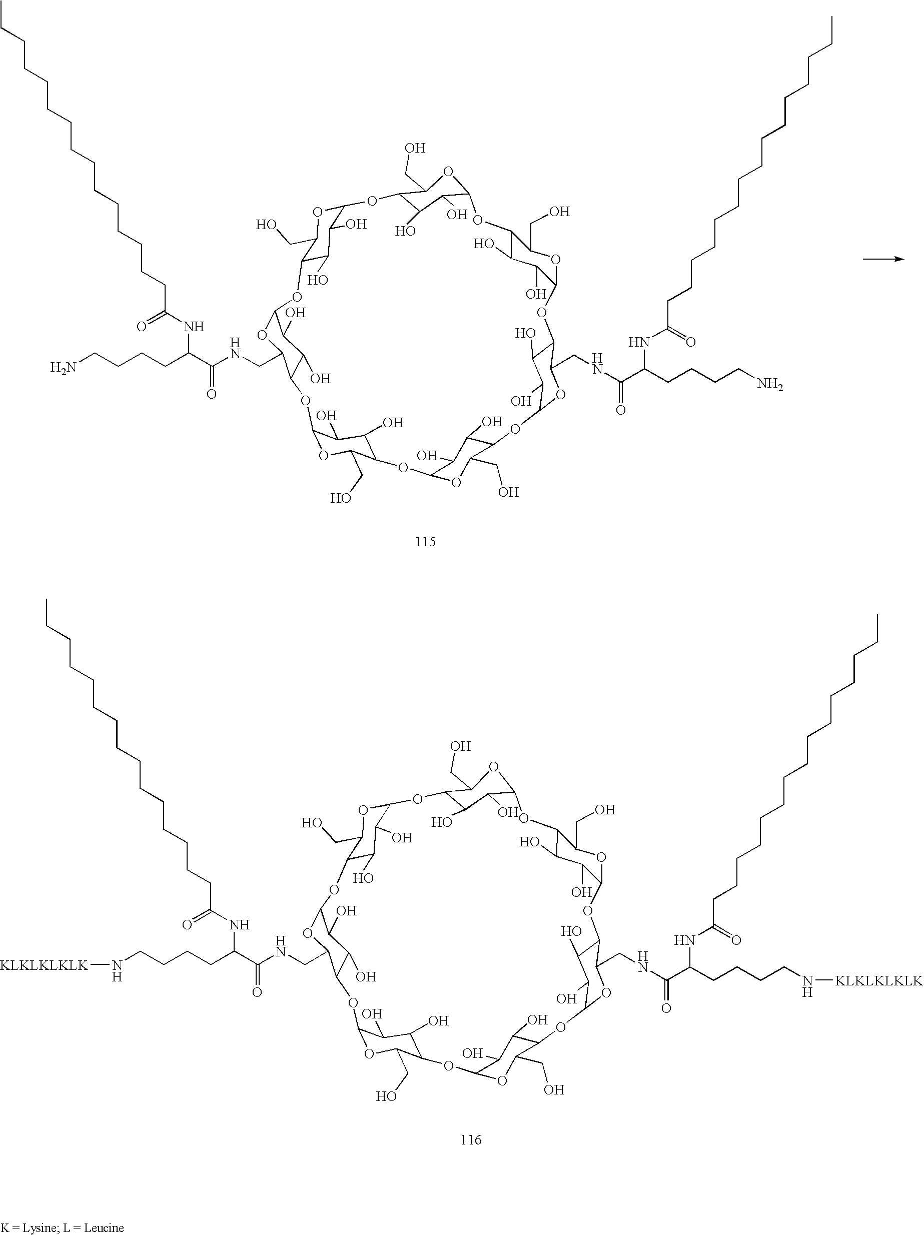

Compound 116 was prepared after HPLC purification by the general procedure H between cyclodextrin 115 and Fmoc-Lys(Boc)-Leu-Lys(Boc)-Leu-Lys(Boc)-Leu-Lys(Boc)-Leu-Lys(Boc)-OH and the general procedures I and J to remove Fmoc and Boc groups. ¹H-NMR (300 MHz, D₂O): δ 4.7-5.0 (m, 7H), 2.7-4.4 (m, 90H), 1.2-1.8 (m, 96H). 1.0-1.25 (m, 29H), 0.75-1.00 (m, 48H); MS m/z Calcd. For C₁₉₄H₃₆₄N₃₄O₅₅ 4053, Found 4056.

### Example 27

### siRNA Binding Assay

siRNA binding—The relative binding affinity for each TCPC compound was monitored by both gel mobility shift and dye exclusion (see Morgan, A. R., Evans, D. H., Lee J. S., and Pulleyblank, D. E. 1979. Review: Nucl. Acids Res. 1979, 7, 571-594.) assays. Gel mobility shift assays were performed essentially as described as follows (see Parker, G. S., Eckert, D. M., and Bass, B. L. RNA. 2006, 12, 807-818.): Samples of ten or twenty-microliter scale with 50 pM end ³²P-labeled siRNA and various TCPC concentrations were incubated for 15 min at room temperature in a buffer containing a final concentration of 20 mM Tris pH 8.0, 150 mM NaCl, and 10% glycerol. Gel shifts assays of these samples were applied on 10% native gels electrophoresed at 4° C. RNA complexes were visualized using a Molecular Dynamics Typhoon PhosphorImager and apparent affinities were calculated as previously described. (see Parker, G. S., Eckert, D. M., and Bass, B. L. RNA. 2006, 12, 807-818.)

siRNA bound by TCPC is refractory to SYBR Green II (Invitrogen) dye intercalation, resulting in a reduction of fluorescence intensity. The dye exclusion assay monitors this reduction as a function of increasing TCPC concentration. TCPC-siRNA complexes were prepared in TE buffer by titrating siRNA with increasing amounts of TCPC in Greiner Bio-One black 96-well plates. Final concentrations were 10 nM siRNA and 17 pM-1 μM TCPC in a final volume of 100 μl. Binding was allowed to equilibrate for 20 minutes before the addition of 10 μl of a 1:8000 SYBR Green II dilution in TE buffer. Fluorescence was measured using a SpectraMax M5 fluorometer (Molecular Devices) by exciting at 254 nm while monitoring emission at 520 nm. Relative affinities were obtained from resulting binding curves analyzed using GraphPad Prism software.

### Example 28

### Luciferase Knockdown Assay

Human Embryonic Kidney cells (HEK-293) were obtained from the American Type Culture Collection (Mannasas, Va.) and grown in DMEM medium supplemented with 10% fetal bovine serum. Luciferase expressing clones of HEK-293 were generated by transfection with the luciferase mammalian expression vector pGL4 (Promega corp., Madison, Wis.) and drug selected on 500 uG/ml of neomycin. The selected pool was then single cell cloned by limiting dilution. Luciferase expression of individual clone was determined using the Steady Glo assay kit (Promega corporation). A high expression clone, #11, was selected for use in knockdown assays.

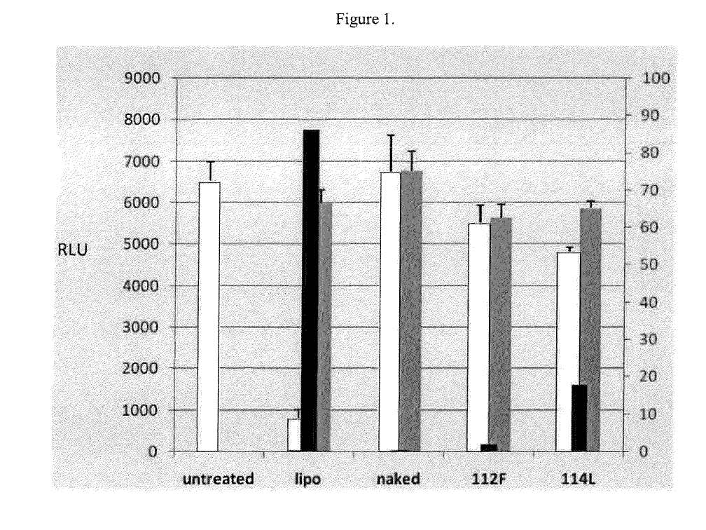

The siRNA sequence encoding siRNA knockdown sequence (SEQ ID No.1: CCUACGCCGAGUACUUCGACU (sense) and SEQ ID No. 2: UCGAAGUACUCGGCGUAGGUA (antisense)) for luciferase mRNA were purchased from Integrated DNA technologies (San Diego, Calif.). The siRNAs were annealed at 65° C. for 5 minutes and allowed to cool to room temperature to form 19 bp duplexes with 2 bp overhangs. Control siRNAs using scrambled luciferase knockdown sequence were also obtained from integrated DNA technologies for use as a negative control. For knock down assays, HEK 293-luciferase clone 11 cells were plated at a density of 5000 cells per well in 96 well white assay plates with clear bottoms (corning costar) in 100 μl growth medium per well. For positive control wells, 25 pmol per well of luciferase knockdown siRNA was complexed with lipofectamine 2000 (Invitrogen corp., San Diego, Calif.) as per manufacturer's recommendations. Negative control wells received equals amounts of scrambled sequence complexed with lipofectamine 2000. Test wells received 25 pmols luciferase knockdown siRNA or scrambled siRNA complexed with 125 pmols of test compound diluted in 50 μL of DMEM medium to yield a final test volume of 150 μL per well. After a 72 h incubation of HEK-luciferase cells with test complexes in a 5% CO₂, 37° C. incubator, luciferase expression was measured in a plate luminometer (Molecular Devices M5) using the steady glo luciferase assay kit as per manufacturers recommendations. Percent knockdown was calculated by comparing the luciferase expression of the test compound complexed with the luciferase knockdown sequence versus the luciferase expression of the test compound complexed with the scrambled knockdown sequence. The results is shown in FIG. 1.

siRNA binding, internalization and the luciferase knockdown for the exemplary compounds are scored and listed in Table 1.

TABLE 1
Compound Binding, Internalization and Knock Down Score

| Compound No. | Binding Affinity | Internalization | Knockdown |
|---|---|---|---|
| 3 | − | | |
| 23b | −/+ | | |
| 23c | + | | |
| 25c | ++ | | |
| 25e | + | | |
| 25h | − | | |
| 25i | − | | |
| 25j | ++ | | |
| 25k | + | | |
| 25l | + | | |
| 25m | ++ | | |
| 25n | + | | |
| 25o | ++ | | |
| 25p | + | | |
| 25q | + | | |
| 25r | + | | |
| 25s | + | | |
| 25w | + | | |
| 25y | + | | |
| 26 | −/+ | | |
| 27 | + | | |
| 28 | + | | |
| 29 | + | | |
| 30 | + | | |
| 31 | ++ | | |
| 32 | ++ | + | |
| 33 | + | | |
| 34 | ++ | | |
| 35 | ++ | + | + |
| 36 | ++ | | |
| 37 | +++ | | |
| 38 | +++ | | |
| 39 | +++ | | |
| 40 | +++ | | |
| 41 | +++ | | |
| 42 | ++ | | |
| 43 | ++ | | |
| 44 | +++ | + | + |
| 45 | ++ | | |
| 46 | +++ | + | |
| 47 | ++ | | |
| 48 | ++ | | |
| 49 | ++ | | |
| 50 | + | | |
| 51 | ++ | | |
| 52 | + | | |
| 53 | ++ | | |
| 54 | + | + | ++ |
| 55 | ++ | | |
| 56 | ++ | | |
| 57 | + | | |
| 58 | + | ++ | + |
| 59 | ++ | + | ++ |
| 60 | +++ | − | ++ |
| 61 | ++ | − | |
| 62 | ++ | | |
| 63 | ++ | + | + |
| 64 | + | | |
| 65 | ++ | | |
| 66 | ++ | +++ | + |
| 67 | ++ | + | |
| 68 | ++ | + | ++ |
| 69 | ++ | ++ | +++ |
| 70 | +++ | + | +++ |
| 71 | +++ | − | |
| 72 | +++ | | |
| 73 | +++ | + | |
| 74 | +++ | + | + |
| 75 | +++ | + | |
| 76 | +++ | − | |
| 77 | ++ | + | ++ |
| 78 | +++ | ++ | + |
| 79 | +++ | ++ | + |
| 80 | ++ | ++ | ++ |
| 81 | +++ | +++ | ++ |
| 82 | ++ | +++ | |
| 83 | ++ | ++ | ++ |
| 84 | ++ | ++ | + |
| 85 | ++ | | |
| 86 | + | + | +++ |
| 87 | ++ | ++ | +++ |
| 88 | + | +++ | +++ |
| 89 | ++ | | |
| 90 | + | | |
| 91 | ++ | | |
| 92 | ++ | | |
| 93 | + | | |
| 94 | ++ | | |
| 95 | −/+ | | |
| 96 | + | | |
| 97 | + | | |
| 98 | + | | |
| 99 | +++ | | |
| 100 | + | | |
| 101 | + | | |
| 107 | + | | |
| 108d | + | + | ++ |
| 109 | + | | |
| 110 | + | | |
| 111a | + | + | +++ |
| 111b | ++ | + | − |
| 111c | +++ | | |
| 111d | ++ | | − |
| 112b | +++ | ++ | ++ |
| 112c | +++ | + | + |
| 112d | +++ | ++ | +++ |
| 112e | +++ | +++ | ++ |
| 112f | +++ | | + |
| 114a | ++ | | |
| 114b | ++ | | |
| 114c | + | + | ++ |
| 114d | + | | |
| 114e | + | ++ | +++ |
| 114f | ++ | + | ++ |
| 114h | ++ | + | +++ |
| 114i | ++ | + | ++ |
| 114j | +++ | + | ++ |
| 114k | +++ | ++ | ++ |
| 114l | + | | ++ |
| 114o | ++ | | − |
| 116 | + | +++ | |

The results demonstrate that the invention constructs comprising a variety of cationic arms and linkers are capable of binding to anionic charged molecules (e.g. siRNA) and are useful for delivering such anionic charged molecule to a cell.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope. Changes therein and other uses will occur to those skilled in the art, which are encompassed within the spirit of the invention, are defined by the scope of the claims.

Definitions provided herein arc not intended to be limiting from the meaning commonly understood by one of skill in the art unless indicated otherwise.

The inventions illustratively described herein may suitably be practiced in the absence of any element or elements, limitation or limitations, not specifically disclosed herein. Thus, for example, the terms “comprising”, “including,” containing“, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A construct represented by formula I:
CA¹-L¹-CD-L²-CA² (1)
wherein,
CD=cyclodextrin;
L¹, L²=linker; and
CA¹, CA²=cationic arm.

2. The construct of claim 1, wherein said cyclodextrin is alpha, beta or gamma cyclodextrin.

3. The construct of claim 2, further comprising a bio-recognition molecule.

4. The construct of claim 1, wherein each linker is independently selected from the group consisting of a covalent bond, a disulfide linkage, a protected disulfide linkage, an ether linkage, a thioether linkage, a sulfoxide linkage, a sulfonate linkage, an ester linkage, an amide linkage, a carbamate linkage, a dithiocarbamate linkage, an amine linkage, a hydrazone linkage, a sulfonamide linkage, an urea linkage, and combinations thereof

5. The construct of claim 4, wherein each linker is covalently linked to the 6-position of A,D-rings, A,C-rings or A,E-rings of said cyclodextrin.

6. The construct of claim 1, wherein each cationic arm comprises a plurality of residues selected from amines, guanidines, amidines, N-containing heterocycles, or combinations thereof

7. The construct of claim 1, wherein each cationic arm comprises a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof.

8. The construct of claim 7, wherein each cationic arm comprises an oligomer independently selected from the group consisting of oligopeptide, oligoamide, cationically functionalized oligoether, cationically functionalized oligosaccharide, oligoamine, oligoethyleneimine, and combinations thereof

9. The construct of claim 8, wherein said oligomer is an oligopeptide.

10. The construct of claim 9, wherein substantially all of the amino acid residues of said oligopeptide are capable of forming positive charges.

11. The construct of claim 10, wherein said oligopeptide comprises 3 to 50 amino acids.

12. The construct of claim 1, wherein
CD=beta-cyclodextrin;
L¹, L²=linker; and
CA¹, CA² comprise independently an oligopeptide;
wherein each linker is covalently linked to the 6-position of A,D-rings of said beta-cyclodextrin.

13. A complex comprising a construct of claim 1 associated with an anionic charged molecule.

14. The complex of claim 13, wherein said anionic charged molecule is selected from the group consisting of a double-stranded nucleic acid, hairpin nucleic acid, single-stranded DNA, double-stranded DNA, single-stranded RNA, double-stranded RNA, and oligonucleotide comprising non-natural monomers.

15. A composition comprising a pharmaceutical excipient, an anionic charged molecule and a construct of claim 1, or a pharmaceutically acceptable ester, salt, or hydrate thereof.

16. A method of attenuating expression of a target gene in treated cells comprising delivering a construct of claim 1 and a double-stranded or hairpin nucleic acid to said cell.

17. A method for delivering an anionic charged molecule to a cell, said method comprising:
a) binding non-covalently a construct of claim 1 to said anionic charged molecule to form a complex; and
b) contacting said cell with said complex; wherein said anionic charged molecule is taken up by said cell.

18. A method for delivering an anionic charged molecule to a cell, said method comprising contacting said cell with a complex prepared by binding non-covalently a
construct of claim 1 to said anionic charged molecule, wherein said anionic charged molecule is taken up by said cell.

19. A method for stabilizing an anionic charged molecule in vivo or for reducing the susceptibility of an anionic charged molecule to self-aggregation, said method comprising contacting said anionic charged molecule with the construct of claim 1.

20. A method for (a) increasing the temperature of hybrid dissociation of a double-stranded hairpin nucleic acid, (b) reducing the susceptibility of a double-stranded or hairpin nucleic acid to digestion by enzymatic nuclease or (c) reducing the susceptibility of a double-stranded or hairpin nucleic acid to hydrolysis of the phophodiester backbone, said method comprising contacting said nucleic acid with a construct of claim 1.

21. A method for preparing a construct of formula I comprising:
a) covalently attaching linkers L¹ and L² to a cyclodextrin; and
b) covalently attaching cationic arms CA¹ and CA² to L¹ and L², respectively or
a′) covalently attaching a first linker to a first cationic arm to form L¹-CA¹ and a second linker to a second arm to form L²-CA²; and
b′) covalently attaching L¹-CA¹ and L²-CA² to a cyclodextrin.

22. A method comprising reacting an optionally substituted 6-perbenzyl cyclodextrin with a hydride reducing agent to produce a 6^{A},6^{D} or a 6^{A},6^{E} dihydroxyl cyclodextrin.

23. The method claim 22, wherein said optionally substituted 6-perbenzyl cyclodextrin is 6-per-(p-methoxylbenzyl) cyclodextrin.

24. The method claim 23, wherein said hydride reducing agent is an aluminum hydride reducing agent.

25. The method of claim 24, wherein said aluminum hydride reducing agent is diisobutylaluminium hydride.

26. A compound represented by formula II: 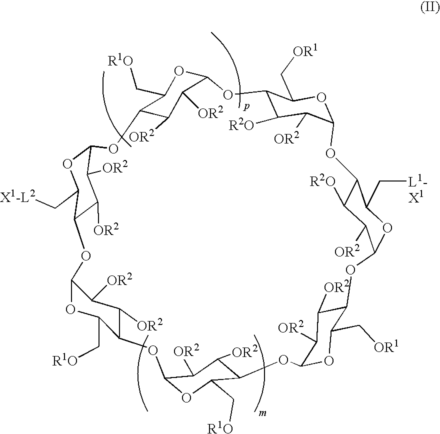
wherein:
m is 0, 1 or 2;
p is 1 or 2, provided when p is 2, m is 1;
L¹ and L² are linkers independently selected from the group consisting of a covalent bond, a disulfide linkage, a protected disulfide linkage, an ether linkage, a thioether linkage, a sulfoxide linkage, a sulfonate linkage, an ester linkage, an amide linkage, a carbamate linkage, a dithiocarbamate linkage, an amine linkage, a hydrazone linkage, a sulfonamide linkage, an urea linkage, and combinations thereof;
R¹ is selected from the group consisting of hydrogen, alkyl, substituted alkyl, acyl, carbamoyl and silyl;
R² is selected from the group consisting of hydrogen, alkyl, substituted alkyl, and acyl;
X¹ and X² are independently displaceable functional groups;
with the proviso that R₁ and R2 are not the same; said ether linkage is not p-(allyloxy)phenyl ether linkage; and said amide linkage is not p-(allyloxy)benzoyl amide linkage.
